(19) United States
(12) Patent Application Publication
(10) Pub. No.: US 2024/0261312 A1
(43) Pub. Date: Aug. 8, 2024

(54) Method and compositions for treating animal viral infections

(71) Applicant: PHOENIX BIOTECHNOLOGY, INC., SAN ANTONIO, TX (US)

(72) Inventors: Robert A. NEWMAN, St. Helena Island, SC (US); Christopher C.L. CHASE, Brookings, SD (US); Jose R. MATOS, Plano, TX (US)

(73) Assignee: PHOENIX BIOTECHNOLOGY, INC., SAN ANTONIO, TX (US)

(21) Appl. No.: 18/438,667

(22) Filed: Feb. 12, 2024

Related U.S. Application Data
(63) Continuation-in-part of application No. PCT/US2022/038505, filed on Jul. 27, 2022.
(60) Provisional application No. 63/337,804, filed on May 3, 2022.
(60) Provisional application No. 63/233,578, filed on Aug. 16, 2021.

(51) Int. Cl. A61K 31/7048; A61K 35/74; A61K 36/24; A61K 45/06; A61P 31/16

(52) U.S. Cl. CPC A61K 31/7048; A61K 35/74; A61K 36/24; A61K 45/06; A61P 31/16

(57) ABSTRACT

A method of treating viral infection in an animal is provided. Oleandrin or digoxin are administered to treat viral infection is caused by any of the following virus families: Arterviridae, Astroviridae, Bomaviridae, Circoviridae, Coronaviridae, Chordopoxvirinae, Flaviviridae, Herpesviridae, Orthomyxoviridae, Papillomaviridae, Papovaviridae, Paramyxoviridae, Parvoviridae, Picornaviridae, Poxviridae, Reoviridae, Retroviridae, Rhabdoviridae, and Togaviridae. An antiviral composition may be administered to prevent the disease state of the viral infection. Domesticated, companion, and livestock animals can be treated.

### CROSS-REFERENCE TO EARLIER FILED APPLICATIONS

The present application claims the benefit of priority and is a continuation-in-part of international application No. PCT/US2022/038505 filed Jul. 27, 2022, which claims the benefit of priority of applications No. 63/337,804 filed May 3, 2022, and No. 63/233,578 filed Aug. 16, 2021, the entire disclosures of which are hereby incorporated by reference.

### FIELD OF THE INVENTION

The present invention concerns a method of treating animal viral infection(s) by administration of a cardiac glycoside, in particular, oleandrin, to an animal in need thereof. Arterviridae, Flaviviridae, Paramyxoviridae, Picomaviridae, Chordopoxvirinae, Poxviridae, Coronaviridae, Papillomaviridae, Rhabdoviridae, Parvoviridae, Orthomyxoviridae, Reoviridae, Astroviridae, and Circoviridae family viral infections may be treated. In particular, bovine coronavirus (BCV), porcine coronavirus (PCV), bovine viral diarrhea virus (BVDV), bovine respiratory syncytial virus (BRSV), and porcine reproductive and respiratory syndrome virus (PRRSV) can be treated.

### BACKGROUND OF THE INVENTION

Oleandrin is a cardiac glycoside obtained by extraction from Nerium oleander (Nerium odorum) plant. It is widely recognized in the animal industry that consumption of the plant material is toxic to animals and on occasion may result in fatal poisoning. (Rubini et al., “A probable fatal case of oleander (Nerium oleander) poisoning on a cattle farm: a new method of detection and quantitation of the oleandrin toxin in rumen” in Toxins (2019), 11, 442; Ceci et al., “Outbreak of oleander (Nerium oleander) poisoning in dairy cattle: clinical and food safety implications” in Toxins (2020), 12, 471; Aslani et al., “Clinical and pathological aspects of experimental oleander (Nerium oleander) toxicosis in sheep” in Vet. Res. Commun. (2004), 28, 609-616; Barbosa et al., “Toxicity in goats caused by oleander (Nerium oleander)” in Res. Vet. Sci. (2008), 85, 279-281; Soto-Blanco et al., “Acute cattle intoxication from Nerium oleander pods” in Trop. Anim. Health Prod. (2006), 38, 451-454).

Oleander is considered the most important cause of livestock poisoning in South Africa. Accidental intoxications have been reported in horses, donkeys, cattle, camelids (alpaca and llama), dogs, cats and pet birds. Mydriasis in animals, after oleander ingestion, is also observed in relation to the increased sympathetic tone. For this reason, no therapeutic products derived from the plant have been developed for use in animals such as commercial animals or livestock, e.g. horses, cows, pigs, goats, sheep, poultry, etc.

Animal viruses are subdivided into seven groups: DNA viruses (Group I and II), RNA viruses (Group III, IV, and V), and RT viruses (Group VI and VII). Group I is represented by viruses containing a double-stranded DNA genome. Group I viruses (adenovirus, herpes virus, papovavirus, poxvirus) synthesize mRNA by transcription from the DNA genome template. Group I viruses cause respiratory disease, conjunctival pneumonia, acute hemorrhagic cystitis, or acute gastroenteritis. Group II (parvovirus) is represented by viruses containing a single-stranded DNA genome. Group II viruses first convert their single-stranded DNA genome to double-stranded DNA, which is then used as a template for mRNA transcription. Group II is represented by viruses containing a double-stranded RNA genome. Group III viruses synthesize mRNA by transcription from their double-stranded RNA template. Group IV is represented by viruses containing a positive-sense single-stranded RNA genome. Group IV viruses utilize the genomic RNA directly as mRNA (denoted by dotted lines in the figure). Group V is represented by viruses containing a negative-sense single-stranded RNA genome. Group V viruses synthesize mRNA by transcription from their RNA genome template. Group VI and VII are “reverse transcribing (RT) viruses” viruses. Although they have either RNA or double-stranded DNA genome, these RT viruses are not classified as either RNA or DNA viruses. An important feature that is shared by the RT viruses is that the viral DNAs are synthesized via reverse transcription. Note that although Group VI viruses contain a single-stranded RNA genome, the genomic RNA does not serve as mRNA, unlike those of Group IV. Group VII viruses contain a double-stranded DNA genome.

Negative-sense single-stranded enveloped RNA viruses ((−)-(ss)-envRNAV) include those in the Arenaviridae family, Bunyaviridae family (Bunyavirales order), Filoviridae family, Orthomyxoviridae family, Paramyxoviridae family, and Rhabdoviridae family. Positive-sense single-stranded enveloped RNA virus (+)-(ss)-envRNAV include Coronaviridae family (human and animal pathogen), Flaviviridae family (human and animal pathogen), Togaviridae family (human and animal pathogen), Arterviridae family (animal pathogen), Retroviridae family.

Viruses that are virulent to animals of domestic or commercial importance are common in chickens, turkeys, pigs, cows, horses, sheep, goats, horses, buffalo, pigeons, etc. Exemplary viruses include porcine circovirus type-2 (PCV2), porcine reproductive and respiratory syndrome (PRRS) virus, bovine viral diarrhea virus (BVD) virus, bovine herpes virus type 1 virus (BHV-1, e.g. infectious bovine rhinotracheitis (IBR)), bovine papillomavirus, lyssavirus (rabies, a Rhabdovirus), Foot and Mouth Disease virus (FMD; aphthovirus of the family Picornaviridae; e.g. serotypes A, O, C, SAT1,SAT2, SAT3, Asia1), lumpy skin disease virus (Capripoxvirus of the Poxviridae family), African horse sickness virus (Reoviridae), sheeppox virus and goatpox virus (subfamily Chordopoxviridae, genus Capripoxvirus), equine influenza virus, equine infectious anemia virus, equine arteritis virus, African swine fever virus, classical swine fever virus, Nipah virus, swine vesicular disease virus, transmissible gastroenteritis virus of swine, avian infectious bronchitis virus, infectious laryngotracheitis virus (avian), duck hepatitis virus, avian influenza virus, infectious bursal disease virus (Gumboro), Marek's disease virus (visceral leukosis; Herpes virus), virulent Newcastle disease virus (vNDV, Paramyxoviridae, genus Avulavirus), avian metapneumovirus (in turkey), avian influenza virus, Poultry Enteritis Mortality Syndrome (PEMS in turkey), columbid alphaherpesvirus-1 (CoHV-1), avian nephritis, arbovirus infections, turkey viral hepatitis, avian encephalomyelitis, avian hepatitis E virus, chicken cholera, fowl pox, fowl cholera, hemorrhagic enteritis in turkeys, canine parvovirus type 1 or type 2, infectious canine hepatitis (ICH, adenovirus 1), canine herpes, canine distemper virus (Morbillivirus), rotavirus intestinal viral in dogs, porcine herpesvirus 1 (pseudorabies, Aujeszky's disease), canine influenza, canine parainfluenza virus, feline herpes virus, feline immunodeficiency virus, feline parvovirus, feline infectious peritonitis virus, feline influenza virus, feline calicivirus, feline leukemia virus, feline viral rhinotracheitis, feline coronavirus, feline rotavirus, feline astrovirus, Torque teno sus virus (TTSuV), Porcine teschovirus (PTV), Porcine bocavirus 1 (PBoV1), swine influenza virus (e.g. type A), porcine endemic diarrhea virus (PEDV), porcine deltacoronavirus, and others. Many of these viruses have no suitable antiviral treatments.

Coronavirus (CoV) is the common name for Coronaviridae. In animals, CoV causes respiratory infections, e.g. bovine coronavirus (BCV). Bovine coronavirus (BCV) is a viral cause of calf enteritis (inflammation of the intestine usually accompanied by diarrhea). The virus infects the intestines and/or upper respiratory tract of calves and contributes to the development of pneumonia. It is also the cause of Winter Dysentery in adult housed cattle. Bovine coronavirus has been found in cattle worldwide. The incidence of BCV varies in different parts of the world but published and annual reports indicate that BCV causes 15-30% of all calf enteritis cases. Incidence may be underestimated because many laboratories around the world are not equipped with BCV antigen detection methods. Clinical signs include diarrhea, sometimes with hematochezia or melaena, rumen atony, anorexia or a reduced appetite, weight loss or reduced weight gain, decreased milk yield and dehydration and depression. Respiratory signs may include serous nasal discharge, progressing to purulent if secondary bacterial infection is present, coughing, dyspnea and tachypnoea. A substantial need remains for effective antiviral treatments (compositions and methods) against BCV.

Bovine viral diarrhea (BVDV) is a viral disease that affects cattle worldwide. Caused by a pestivirus, it gives rise to significant economic losses in both dairy and beef cattle through its effects on production and reproduction. Bovine viral diarrhea virus can lead to a variety of clinical outcomes that range from subclinical infections to the more severe presentations including abortion, infertility, and the fatal mucosal disease. The condition is highly immuno-suppressive and secondary respiratory and enteric complications often occur. A substantial need remains for effective antiviral treatments (compositions and methods) against BVDV.

Bovine Respiratory Syncytial Virus (BRSV) is a respiratory condition in cattle. It replicates in nasal epithelium and then disperses throughout the upper respiratory tract to the bronchial tree. Here, syncytia form and further spread into the bronchioles occurs. Outbreaks of RSV associated disease usually occur associated with winter housing and also during periods of stress such as mixing of calves and transport. The virus can contribute to calf enzootic pneumonia. Vaccines are available but are not typically very effective. A substantial need remains for effective antiviral treatments (compositions and methods) against BRSV.

Porcine reproductive and respiratory syndrome (PRRS or PRRSV) is a viral disease characterized by two overlapping clinical presentations, reproductive impairment or failure in breeding animals, and respiratory disease in pigs of any age. PRRS is the most economically significant disease to affect US swine production since the eradication of classical swine fever (CSF). Worldwide, PRRS is the most economically important infectious disease of pigs. Porcine reproductive and respiratory syndrome virus (PRRSV) occurs in all age groups. Reproductive impairment or failure, more obvious in sows or gilts, also affects some boars. The respiratory syndrome is seen more often in young growing pigs but also occurs in naïve finishing pigs and breeding stock. Although reported initially in only a few countries in the late 1980s, PRRS now occurs worldwide in most major swine-raising countries. PRRS is prevalent in the United States and exists both in epidemic and endemic forms. There is no single successful strategy for control of PRRS, largely because of virus variation, large swine populations, and unresolved issues of transmission. A substantial need remains for effective antiviral treatments (compositions and methods) against PRRSV.

The antiviral of specific compounds against specific viruses is unpredictable. In some cases, a compound may be found active against a first virus but inactive against a second virus. Moreover, viruses unpredictably develop resistance to antiviral drugs (Kirwin et al. “Antiviral drug resistance as an adaptive process” in Virus Evol. (January 2106), 2(1), 1-10). Development of drug resistance has been found for amantadine, oseltamivir, and other drugs. Drug resistant strains of HIV, influenza, hepatitis B, polio, hepatitis C, HSV-2, and others.

Nerium oleander, a member of the Nerium species, is an ornamental plant widely distributed in subtropical Asia, the southwestern United States, and the Mediterranean. Its medical and toxicological properties have long been recognized. In humans, it has been proposed for use, for example, in the treatment of hemorrhoids, ulcers, leprosy, snake bites, cancers, tumors, neurological disorders, warts, and cell-proliferative diseases. Zibbu et al. (J. Chem. Pharm. Res. (2010), 2(6), 351-358) provide a brief review on the chemistry and pharmacological activity of Nerium oleander.

Extraction of components from plants of Nerium species has traditionally been carried out using boiling water, cold water, supercritical fluid, or organic solvent.

ANVIRZEL™ (U.S. Pat. No. 5,135,745 to Ozel) contains the concentrated form or powdered form of the hot-water extract of Nerium oleander. Muller et al. (Pharmazie. (1991) September 46(9), 657-663) disclose the results regarding the analysis of a water extract of Nerium oleander. They report that the polysaccharide present is primarily galacturonic acid. Other saccharides include rhamnose, arabinose and galactose. Polysaccharide content and individual sugar composition of polysaccharides within the hot water extract of Nerium oleander have also been reported by Newman et al. (J. Herbal Pharmacotherapy, (2001) vol 1, pp. 1-16). Compositional analysis of ANVIRZEL™, the hot water extract, was described by Newman et al. (Anal. Chem. (2000), 72(15), 3547-3552). U.S. Pat. No. 5,869,060 to Selvaraj et al. pertains to extracts of Nerium species and methods of production. To prepare the extract, plant material is placed in water and boiled. The crude extract is then separated from the plant matter and sterilized by filtration. The resultant extract can then be lyophilized to produce a powder. U.S. Pat. No. 6,565,897 (U.S. Pregrant Publication No. 20020114852 and PCT International Publication No. WO 2000/016793 to Selvaraj et al.) discloses a hot-water extraction process for the preparation of a substantially sterile water extract. Ishikawa et al. (J. Nutr. Sci. Vitaminol. (2007), 53, 166-173) discloses a hot water extract of Nerium oleander and fractionation thereof by liquid chromatography using mixtures of chloroform, methanol, and water. They also report that extracts of the leaves of N. oleander have been used to treat Type II diabetes. US20060188585 published Aug. 24, 2006 to Panyosan discloses a hot water extract of Nerium oleander. U.S. Ser. No. 10/323,055 issued Jun. 18, 2019 to Smothers discloses a method of extracting plant material with aloe and water to provide an extract comprising aloe and cardiac glycoside. US20070154573 published Jul. 5, 2007 to Rashan et al. discloses a cold-water extract of Nerium oleander and its use.

Erdemoglu et al. (J. Ethnopharmacol. (2003) November 89(1), 123-129) discloses results for the comparison of aqueous and ethanolic extracts of plants, including Nerium oleander, based upon their anti-nociceptive and anti-inflammatory activities. Fartyal et al. (J. Sci. Innov. Res. (2014), 3(4), 426-432) discloses results for the comparison of methanol, aqueous, and petroleum ether extracts of Nerium oleander based upon their antibacterial activity.

Organic solvent extracts of Nerium oleander are also disclosed by Adome et al. (Afr. Health Sci. (2003) August 3(2), 77-86; ethanolic extract), el-Shazly et al. (J. Egypt Soc. Parasitol. (1996), August 26(2), 461-473; ethanolic extract), Begum et al. (Phytochemistry (1999) February 50(3), 435-438; methanolic extract), Zia et al. (J. Ethnolpharmacol. (1995) November 49(1), 33-39; methanolic extract), and Vlasenko et al. (Farmatsiia. (1972) September-October 21(5), 46-47; alcoholic extract). Turkmen et al. (J. Planar Chroma. (2013), 26(3), 279-283) discloses an aqueous ethanol extract of Nerium oleander leaves and stems. U.S. Pat. No. 3,833,472 issued Sep. 3, 1974 to Yamauchi discloses extraction of Nerium odorum SOL (Nerium oleander Linn) leaves with water, organic solvent, or aqueous organic solvent, wherein the leaves are heated to 60°*-170° C. and then extracted, and the organic solvent is methanol, ethanol, propyl ether or chloroform.

A supercritical fluid extract of Nerium species is known (U.S. Pat. Nos. 8,394,434, 8,187,644, 7,402,325) and has demonstrated efficacy in treating neurological disorders (U.S. Pat. Nos. 8,481,086, 9,220,778, 9,358,293, US 20160243143A1, U.S. Pat. No. 9,877,979, U.S. Ser. No. 10/383,886) and cell-proliferative disorders (U.S. Pat. Nos. 8,367,363, 9,494,589, 9,846,156), and some viral infections (U.S. Ser. No. 10/596,186, WO 2018053123A1, WO2019055119A1)

Triterpenes are known to possess a wide variety of therapeutic activities. Some of the known triterpenes include oleanolic acid, ursolic acid, betulinic acid, bardoxolone, maslinic acid, and others. The therapeutic activity of the triterpenes has primarily been evaluated individually rather than as combinations of triterpenes.

Oleanolic acid is in a class of triterpenoids typified by compounds such as bardoxolone which have been shown to be potent activators of the innate cellular phase 2 detoxifying pathway, in which activation of the transcription factor Nrf2 leads to transcriptional increases in programs of downstream antioxidant genes containing the antioxidant transcriptional response element (ARE). Bardoxolone itself has been extensively investigated in clinical trials in inflammatory conditions; however, a Phase 3 clinical trial in chronic kidney disease was terminated due to adverse events that may have been related to known cellular toxicities of certain triterpenoids including bardoxolone at elevated concentrations.

Compositions containing triterpenes in combination with other therapeutic components are found as plant extracts. Fumiko et al. (Biol. Pharm. Bull (2002), 25(11), 1485-1487) discloses the evaluation of a methanolic extract of Rosmarimus officinalis L. for treating trypanosomiasis. Addington et al. (U.S. Pat. Nos. 8,481,086, 9,220,778, 9,358,293, US 20160243143 A1) disclose a supercritical fluid extract (SCF; PBI-05204) of Nerium oleander containing oleandrin and triterpenes for the treatment of neurological conditions. Addington et al. (U.S. Pat. No. 9,011,937, US 20150283191 A1) disclose a triterpene-containing fraction (PBI-04711) of the SCF extract of Nerium oleander containing oleandrin and triterpenes for the treatment of neurological conditions. Jager et al. (Molecules (2009), 14, 2016-2031) disclose various plant extracts containing mixtures of oleanolic acid, ursolic acid, betulinic acid and other components. Mishra et al. (PLoS One 2016 25; 11(7):e0159430. Epub 2016 Jul. 25) disclose an extract of Betula utilis bark containing a mixture of oleanolic acid, ursolic acid, betulinic acid and other components. Wang et al. (Molecules (2016), 21, 139) disclose an extract of Alstonia scholaris containing a mixture of oleanolic acid, ursolic acid, betulinic acid and other components. L. e Silva et al. (Molecules (2012), 17, 12197) disclose an extract of Eriope blanchetti containing a mixture of oleanolic acid, ursolic acid, betulinic acid and other components. Rui et al. (Int. J. Mol. Sci. (2012), 13, 7648-7662) disclose an extract of Eucaplyptus globulus containing a mixture of oleanolic acid, ursolic acid, betulinic acid and other components. Ayatollahi et al. (Iran. J. Pharm. Res. (2011), 10(2), 287-294) disclose an extract of Euphorbia microsciadia containing a mixture of oleanolic acid, ursolic acid, betulinic acid and other components. Wu et al. (Molecules (2011), 16, 1-15) disclose an extract of Ligustrum species containing a mixture of oleanolic acid, ursolic acid, betulinic acid and other components. Lee et al. (Biol. Pharm. Bull (2010), 33(2), 330) disclose an extract of Forsythia viridissima containing a mixture of oleanolic acid, ursolic acid, betulinic acid and other components.

Oleanolic acid (O or OA), ursolic acid (U or UA) and betulinic acid (B or BA) are the three major triterpene components found in PBI-05204 (PBI-23; a supercritical fluid extract of Nerium oleander) and PBI-04711 (a triterpene-containing fraction 0-4 of PBI-05204). Van Kanegan et al. previously reported (Nature Scientific Reports (May 2016), 6:25626. doi: 10.1038/srep25626) on the contribution of the triterpenes toward efficacy by comparing their neuroprotective activity in a brain slice oxygen glucose deprivation (OGD) model assay at similar concentrations. PBI-05204 (PBI) and PBI-04711 (Fraction 0-4) were found to provide neuroprotective activity.

Extracts of Nerium species are known to contain many different classes of compounds: cardiac glycosides, glycones, steroids, triterpenes, polysaccharides and others. Specific compounds include oleandrin; neritaloside; odoroside; oleanolic acid; ursolic acid; betulinic acid; oleandrigenin; oleaside A; betulin (urs-12-ene-3β,28-diol); 28-norurs-12-en-3β-ol; urs-12-en-3β-ol; 3β,3β-hydroxy-12-oleanen-28-oic acid; 3β,20α-dihydroxyurs-21-en-28-oic acid; 3β,27-dihydroxy-12-ursen-28-oic acid; 3β,13β-dihydroxyurs-11-en-28-oic acid; 3β,12α-dihydroxyoleanan-28,13β-olide; 3β,27-dihydroxy-12-oleanan-28-oic acid; and other components.

Oleandrin, and an extract of Nerium oleander have been shown to prevent the incorporation of the gp120 envelope glycoprotein of HIV-1 into mature virus particles and inhibit viral infectivity in vitro (Singh et al., “Nerium oleander derived cardiac glycoside oleandrin is a novel inhibitor of HIV infectivity” in Fitoterapia (2013) 84, 32-39).

Oleandrin has demonstrated anti-HIV activity but has not been evaluated against many viruses. The triterpenes oleanolic acid, betulinic acid and ursolic acid have been reported to exhibit differing levels of antiviral activity but have not been evaluated against many viruses. Betulinic acid has demonstrated some anti-viral activity against HSV-1 strain 1C, influenza A H7N1, ECHO 6, and HIV-1. Oleanolic acid has demonstrated some anti-viral activity against HIV-1, HEP C, and HCV H strain NS5B. Ursolic acid has demonstrated some anti-viral activity against HIV-1, HEP C, HCV H strain NS5B, HSV-1, HSV-2, ADV-3, ADV-8, ADV-11, HEP B, ENTV CVB1 and ENTV EV71. The antiviral activity of oleandrin, oleanolic acid, ursolic acid and betulinic acid is unpredictable as far as efficacy against specific viruses.

Viruses exist against which oleandrin, oleanolic acid, ursolic acid and/or betulinic acid have little to no antiviral activity, meaning one cannot predict a priori whether oleandrin, oleanolic acid, ursolic acid and/or betulinic acid will exhibit antiviral activity against particular genera of viruses.

Barrows et al. (“A screen of FDA-approved drugs for inhibitors of Zikavirus infection” in Cell Host Microbe (2016), 20, 259-270) report that digoxin demonstrates antiviral activity against Zika virus, but the doses are too high and likely toxic. Cheung et al. (“Antiviral activity of lanatoside C against dengue virus infection” in Antiviral Res. (2014) 111, 93-99) report that lanatoside C demonstrates antiviral activity against Dengue virus.

Even though cardiac glycosides have been demonstrated to exhibit some antiviral activity against a few viruses, the specific compounds exhibit very different levels of antiviral activity against different viruses, meaning that some exhibit very poor antiviral activity and some exhibit better antiviral activity when evaluated against the same virus(es): Emamzadeh-Yazdi (“Antiviral, antibacterial, and cytotoxic activities of South African plants containing cardiac glycosides” in Masters Thesis (Univ. Pretoria), April 2013), Correa Souza et al. (“Na+/K+-ATPase as a target of cardiac glycosides for the treatment of SARS-CoV-2 Infection” in Frontiers Pharma. (2021), 12, 624704), Amarelle et al. (“The antiviral effects of Na,K-ATPase inhibition: a minireview” in Inter. J. Molec. Sci. (2018), 19, 2154), Amarelle et al. (“Cardiac glycosides decrease influenza virus replication by inhibiting cell protein translational machinery” in Am. J. Physiol. Lung Cell Mol. Physiol. (2019), 316, L1094-L1106), Ashbrook (“Antagonism of the sodium-potassium ATPase impairs Chikungunya virus infection” in MBio, (2016), 7(3), e00693-16), Cai et al. (“Digitoxin analogues with improved anti-cytomegalovirus activity” in Med. Chem. Lett. (2014), 5, 395-399), Cheung et al. (“Antiviral activity of lanatoside C against dengue virus infection” in Antivir. Res. (2014), 111, 93-99). Apparently, the antiviral activity of specific cardiac glycosides against species viral species is unpredictable a priori.

Oleandrin has demonstrated antiviral activity against some viruses, but the antiviral activity is unpredictable a priori and even within a particular viral family or viral genus and even across mammalian species: U.S. Ser. No. 10/702,567, U.S. Ser. No. 10/729,735, U.S. Ser. No. 10/596,186, U.S. Ser. No. 11/007,239, U.S. Ser. No. 10/874,704, US 20200206287A1, U.S. Ser. No. 11/013,776, U.S. Ser. No. 10/980,852, WO 2018053123A1, WO 2019055119A1, WO 2020042009A1, Plant et al. (“Antiviral activity of oleandrin and a defined extract of Nerium oleander against SARS-CoV-2” in Biomed. Pharma. (2021), 138, 111457), Newman et al. (“Antiviral effects of Oleandrin” in J. Exp. Pharma. (2020), 12, 503-515), Avci et al. (“Determination of in vitro antiviral activity of Nerium oleander distillate against parainfluenza-3 virus” in Animal Vet. Sci. (2014), 2(5), 150-153) (the distillate does not contain oleandrin), Dey et al. (“Pharmacological aspects of Nerium indicum Mill: a comprehensive review” in Pharmacogn. Rev. (2014), 8(16), 156-162), Singh et al. (Nerium oleander derived cardiac glycoside is a novel inhibitor of HIV infectivity” in Fitoter. (2013), 84, 32-39), Hutchison et al. (The botanical glycoside oleandrin inhibits Human T-cell leukemia virus type-1 infectivity and Env-dependent virological synapse formation” in J. Antivir. Antiretrovir. (2019), 11(3), 184), Plante et al. (“Prophylactic and therapeutic inhibition of in vitro SARS-CoV-2 replication by oleandrin” in bioRxiv (2020), doi: 10.1101/2020.07.15.203489).

In particular, Yang et al. (“Identification of antiviral activity of the cardenolides, Na/K-ATPase inhibitors, against porcine transmissible gastroenteritis virus” in Toxic. Applied Pharma. (2017), 332, 129-137) demonstrate that some cardiac glycosides are active against some coronaviruses in some species but inactive against some coronaviruses in other species. Even within the same animal species, the cardiac glycosides can demonstrate substantially different levels of activity.

A need remains for improved pharmaceutical compositions containing oleandrin (and/or digoxin), optionally together with other compounds obtained from Nerium sp., e.g. oleanolic acid, ursolic acid, betulinic acid or any combination thereof, that are therapeutically active against specific animal viral infections.

### SUMMARY OF THE INVENTION

The invention provides a pharmaceutical composition and method for treating and/or preventing viral infection in an animal; even though, it has been widely known that cardiac glycosides, in particular oleandrin and digoxin, are toxic to animals. The invention also provides a method of treating viral infection in animals by administration of the pharmaceutical composition. The inventors have succeeded in preparing antiviral compositions that exhibit sufficient antiviral activity to justify their use in treating viral infection in animals, while at the same time being administered at doses that are not fatal to the animals. The inventors have developed corresponding treatment methods employing particular dosing regimens.

The invention also provides a prophylactic method of treating an animal at risk of contracting a viral infection, the method comprising chronically administering to the animal one or more doses of an antiviral composition on a recurring basis over an extended treatment period prior to the animal contracting the viral infection, thereby preventing the animal from contracting the viral infection; wherein the antiviral composition comprises oleandrin and/or digoxin. Alternatively, the invention also provides a prophylactic method of treating an animal at risk of having a viral disease, the method comprising chronically administering to the animal one or more doses of an antiviral composition on a recurring basis over an extended treatment period within 0-5 days of the animal having contracted a viral infection that causes said viral disease, thereby preventing the animal from exhibiting symptoms associated with said viral disease; wherein the antiviral composition comprises oleandrin and/or digoxin.

In some embodiments, the antiviral composition is administered to an animal having virally infected cells. In some embodiments, the viral infection is caused by any of the following virus families: Arterviridae, Astroviridae, Bornaviridae, Circoviridae, Coronaviridae, Chordopoxvirinae, Flaviviridae, Herpesviridae, Orthomyxoviridae, Papillomaviridae, Papovaviridae, Paramyxoviridae, Parvoviridae, Picornaviridae, Poxviridae, Reoviridae, Retroviridae, Rhabdoviridae, and Togaviridae.

The animal can be a domestic or livestock animal, e.g. pig, cow, horse, sheep, goat, llama, alpaca, buffalo, deer, elk, giraffe, camel, dog, cat, chicken, turkey, pigeon, duck, pheasant, guinea, or other animal.

Viral infections and diseases that can be treated include Venezuelan Equine Encephalomyelitis (encephalitis) (VEE) virus, Western Equine Encephalomyelitis (encephalitis) (WEE) virus, Eastern Equine Encephalomyelitis (encephalitis) (EEE) virus, bovine coronavirus (BCV), porcine coronavirus (PCV), bovine viral diarrhea virus (BVDV), bovine respiratory syncytial virus (BRSV), porcine reproductive and respiratory syndrome virus (PRRSV), porcine circovirus type-2 (PCV2), bovine herpes virus type 1 (BHV-1, e.g. infectious bovine rhinotracheitis (IBR)), bovine herpes virus type 2 (BHV-2, bovine herpes mamillitis), bovine herpes virus type 3 (BHV-3, catarrhal fever), bovine herpes virus type 5 (BHV-5, encephalitis), bovine papillomavirus, lyssavirus (rabies, a Rhabdovirus), Foot and Mouth Disease virus (FMD; aphthovirus of the family Picornaviridae; e.g. serotypes A, O, C, SAT1,SAT2, SAT3, Asia1), lumpy skin disease virus (Capripoxvirus of the Poxviridae family), cowpox virus, pseudocowpox virus (paravaccinia), bovine leukemia virus, bovine lentivirus, respirovirus (bovine parainfluenza-3 virus), Morbillivirus (rinderpest virus), bovine ephemeral fever virus, vesicular stomatitis virus, African swine fever virus, African horse sickness virus (Reoviridae), sheeppox virus and goatpox virus (subfamily Chordopoxviridae, genus Capripoxvirus), equine influenza virus, equine infectious anemia virus, equine arteritis virus, classical swine fever virus, Nipah virus, swine vesicular disease virus, transmissible gastroenteritis virus of swine, avian infectious bronchitis virus, infectious laryngotracheitis virus (avian), duck hepatitis virus, avian influenza virus, infectious bursal disease virus (Gumboro), Marek's disease virus (visceral leukosis; Herpes virus), virulent Newcastle disease virus (vNDV, Paramyxoviridae, genus Avulavirus), avian metapneumovirus (in turkey), avian influenza virus, Poult Enteritis Mortality Syndrome (PEMS in turkey), columbid alphaherpesvirus-1 (CoHV-1), avian nephritis, arbovirus infections, turkey viral hepatitis, avian encephalomyelitis, avian hepatitis E virus, chicken cholera, fowl pox, fowl cholera, hemorrhagic enteritis in turkeys, canine parvovirus type 1 or type 2, infectious canine hepatitis (ICH, adenovirus 1), canine herpes, canine distemper virus (Morbillivirus), rotavirus intestinal viral in dogs, porcine herpesvirus 1 (pseudorabies, Aujeszky's disease), canine influenza, canine parainfluenza virus, feline herpes virus, feline immunodeficiency virus, feline parvovirus, feline infectious peritonitis virus, feline influenza virus, feline calicivirus, feline leukemia virus, feline viral rhinotracheitis, feline coronavirus, feline rotavirus, feline astrovirus, Torque teno sus virus (TTSuV), Porcine teschovirus (PTV), Porcine bocavirus 1 (PBoV1), swine influenza virus (e.g. type A), porcine endemic diarrhea virus (PEDV), porcine deltacoronavirus, and species and/or variants thereof.

In some embodiments, the invention provides an antiviral composition comprising (consisting essentially of): a) specific cardiac glycoside(s); b) plural triterpenes; or c) a combination of specific cardiac glycoside(s) and plural triterpenes. The specific cardiac glycoside can be selected from the group consisting of oleandrin and digoxin.

One aspect of the invention provides a method of treating viral infection in an animal by chronic administration to the animal of an antiviral composition. The animal is treated by chronically administering to the animal a therapeutically effective amount (therapeutically relevant dose) of the composition, thereby providing relief of symptoms associated with the viral infection or amelioration of the viral infection. Administration of the composition to the animal can begin immediately after infection or any time within zero to about 5 days after infection or at the earliest time after definite diagnosis of infection with virus. The virus can be any virus described herein; however, some viruses are preferred. Chronic administration can be achieved by repeated daily administration of rapid or immediate release dosage form(s) (or composition(s)) or by repeated administration (daily, weekly or monthly) of extended (controlled) release dosage form(s).

Accordingly, the invention also provides a method of treating viral infection in a mammal, the method comprising administering to the mammal one or more therapeutically effective doses of the antiviral composition. The one or more therapeutic dose(s) is(are) not lethal or fatal to the animal. One or more doses are administered on a daily, weekly, and/or monthly basis. One or more doses per day can be administered. The virus can be any virus described herein that is pathogenic to animals.

The invention also provides a method of treating viral infection in an animal in need thereof, the method comprising:
determining whether or not the animal has a viral infection;
indicating administration of antiviral composition;
administering an initial dose of antiviral composition to the animal according to a prescribed initial dosing regimen for a period of time;
periodically determining the adequacy of the animal's clinical response and/or therapeutic response to treatment with antiviral composition; and
if the animal's clinical response and/or therapeutic response is adequate, then continuing treatment with antiviral composition as needed until the desired clinical endpoint is achieved; or
if the animal's clinical response and/or therapeutic response are inadequate at the initial dose and initial dosing regimen, then escalating or deescalating the dose until the desired clinical response and/or therapeutic response in the animal is achieved.

Treatment of the animal with antiviral composition is continued as needed. The dose or dosing regimen can be adjusted as needed until the animal reaches the desired clinical endpoint(s) such as a reduction or alleviation of specific symptoms associated with the viral infection. Determination of the adequacy of clinical response and/or therapeutic response can be conducted by a clinician familiar with viral infections.

The individual steps of the methods of the invention can be conducted at separate facilities or within the same facility.

The invention provides alternate embodiments, for all the embodiments described herein, wherein the oleandrin is replaced with digoxin or used in combination with digoxin. The methods of the invention may employ oleandrin, digoxin, or a combination of oleandrin and digoxin. Accordingly, oleandrin, digoxin, oleandrin-containing composition, digoxin-containing composition, or oleandrin- and digoxin-containing composition may be used in the methods of the invention. Cardiac glycoside can be taken to mean oleandrin, digoxin or a combination thereof. A cardiac glycoside-containing composition comprises oleandrin, digoxin or a combination thereof.

The invention also provides a method of treating coronavirus infection, in particular an infection of coronavirus that is pathogenic to animals, e.g. BCV infection or PCV infection, the method comprising chronically administering to an animal, having said infection, therapeutically effective doses of cardiac glycoside (cardiac glycoside-containing composition).

Another aspect of the invention provides a method of preventing an animal from exhibiting one or more symptoms associated with viral infection, the method comprising administering to said animal one or more therapeutically effective doses of cardiac glycoside-containing composition, wherein said one or more doses are administered a) prior to said animal being infected with virus; or b) within a period of up to five days, up to four days, up to three days, up to two days, or up to one day of said animal having been infected with virus.

Another aspect of the invention provides a method of preventing a viral infection in an animal from progressing to a disease state or from exhibiting one or more symptoms associated with viral infection, the method comprising administering to said animal one or more therapeutically effective doses of cardiac glycoside-containing composition within a period of up to seven days, up to six days, up to five days, up to four days, up to three days, up to two days, or up to one day of said animal having been infected with the virus. In other words, the composition might not stop the infection from occurring, but it would stop the infection from progressing to the disease state.

In some embodiments, the animal has been in close contact (within six feet) with another animal having a viral infection. Close contact might also be due to said uninfected animal living with, sharing food with, sharing shelter with, sharing air with, or sharing water with a virally infected animal.

The invention also provides a method of treating coronavirus infection, e.g. bovine coronavirus infection or porcine coronavirus infection, by repeatedly administering (through any of the modes of administration discussed herein) to an animal, having said infection, plural therapeutically effective doses of cardiac glycoside (cardiac glycoside-containing composition). One or more doses may be administered per day for one or more days per week and optionally for one or more weeks per month and optionally for one or more months per year.

The equivalent of plural daily doses of cardiac glycoside can be achieved by administering to said animal one or more extended-release dosage forms that release therapeutically effective daily doses of cardiac glycoside throughout a treatment period. Additional means of administering effective daily doses may be achieved through use of dosage forms suitable for use in water, milk, liquid feed, milk substitute, colostrum, colostrum substitute, or solid feed.

The invention also provides a method of treating viral infection in an animal, the method comprising administering to the animal 1-10 doses of cardiac glycoside (cardiac glycoside-containing composition) per day for a treatment period of 2 days to about 2 months. Two to eight, two to six, or four doses can be administered daily during the treatment period. Doses can be administered for 2 days to about 60 days, 2 days to about 45 days, 2 days to about 30 days, 2 days to about 21 days, or 2 days to about 14 days. Said administering can be through any of the modes of administration discussed herein. Systemic administration that provides therapeutically effective plasma levels of oleandrin and/or digoxin in said animal is preferred.

In some embodiments, one or more doses of cardiac glycoside are administered per day for plural days until the viral infection is cured. In some embodiments, one or more doses of cardiac glycoside (cardiac glycoside-containing composition) are administered per day for plural days and plural weeks until the viral infection is cured. One or more doses can be administered in a day. One, two, three, four, five, six or more doses can be administered per day.

A cardiac glycoside-containing composition comprises at least one cardiac glycoside. One or more pharmaceutical excipients are optionally included in said composition. The preferred cardiac glycosides are oleandrin or digoxin. If the cardiac glycoside-containing composition comprises an extract of Nerium sp. or Digitalis lanata plant material(s), the extract can further comprise one or more components extracted from said plant material(s).

In some embodiments, the antiviral composition further comprises at least one cardiac glycoside-metabolism inhibitor, at least one cardiac glycoside-digestion inhibitor, at least one enzyme inhibitor, or a combination thereof.

A veterinary clinician will be able to use known dose escalation or de-escalation protocols to determine a safe and effective dose of oleandrin or digoxin to be administered to an animal.

The maximum tolerated dose (MTD) of cardiac glycoside may vary according to animal species. In some embodiments, a) said animal is a cow and the dose of antiviral composition provides a maximum plasma concentration of digoxin or oleandrin of no more than 1 ng/mL; b) said animal is a pig and the dose of antiviral composition provides a maximum plasma concentration of digoxin or oleandrin of no more than 5 ng/mL; c) said animal is a horse and the dose of antiviral composition provides a maximum plasma concentration of digoxin or oleandrin of no more than 5 ng/mL; d) said animal is a sheep and the dose of antiviral composition provides a maximum plasma concentration of digoxin or oleandrin of no more than 5 ng/mL; or e) said animal is a goat and the dose of antiviral composition provides a maximum plasma concentration of digoxin or oleandrin of no more than 10 ng/mL.

The pharmacokinetics of digoxin in animals allow for determination of suitable doses that provide target plasma concentrations of digoxin. The half-life of digoxin is as follows: a) in cattle—about 7-9 hours; b) in sheep—about 7-8 hours; c) in ewes and lambs-13-15 hours; d) in horses—about 16-18 hours or about 10-23 hours; e) in puppies—about 20-30 hours or about 23 hours; f) in adult dogs—about 4-6 hours; g) in turkeys—about 10-12 hours; h) in cats—about 9-12 hours; i) in calves—about 5-7 hours; and j) in chickens—about 20-30 hours or about 25 hours.

Suitable nonlethal target plasma concentration of digoxin in animals are as follows: a) in horses—less than about 2 ng/ml or about 0.5-2 ng/ml; b) in dogs—less than about 2.5 ng/ml or about 0.5-2.5 ng/ml; c) in cattle—less than about 2.5 ng/ml or about 0.5-2 ng/ml; d) in chickens—less than about 2 ng/ml.

Suitable target doses (one to four times daily) for digoxin in animals are as follows: a) in dogs—less than about 100 microg/kg bodyweight or about 5-60 microg/kg bodyweight; b) in turkeys—less than about 1 mg/kg bodyweight or about 0.05-0.5 mg/kg bodyweight; c) in cattle—less than about 100 micro/kg bodyweight or about 5-50 microg/kg bodyweight; d) in cats—less than about 100 micro/kg bodyweight or 0.5-50 micro/kg bodyweight; e) in horses—less than about 100 micro/kg bodyweight or 0.5-50 micro/kg bodyweight; and f) in chickens—less than 100 micro/kg bodyweight, about 1-50 microg/kg bodyweight, or about 4-20 microg/kg bodyweight.

Where oleandrin is administered to an animal in the form of Nerium species (Nerium sp.), e.g. Nerium oleander or Nerium indicum, leaf material, the amount of dried leaf material will preferably be a) less than 100 mg/Kg bodyweight or less than 50 mg/Kg bodyweight for a cow; b) less than 110 mg/Kg bodyweight for a goat; c) less than 110 mg/Kg bodyweight or less than 250 mg/Kg bodyweight for a sheep.

In some embodiments, the concentration of oleandrin and/or digoxin in the plasma of a treated animal is about 10 ng/mL or less, about 5 ng/mL or less, about 2.5 ng/mL or less, about 2 ng/mL or less, about 1 ng/mL, or about 0.5 ng/mL or less. In some embodiments, the concentration of oleandrin and/or digoxin in the plasma of a treated animal is about 0.0001 ng/mL or more, about 0.0005 ng/mL or more, about 0.001 ng/mL or more, about 0.0015 ng/mL or more, about 0.01 ng/mL or more, about 0.015 ng/mL or more, about 0.1 ng/mL or more, about 0.15 ng/mL or more, about 0.05 ng/mL or more, or about 0.075 ng/mL or more. The daily dose of antiviral composition administered to the animal will be sufficient to provide a plasma concentration of oleandrin or digoxin within at least one of the ranges set forth herein. The invention includes all combinations and selections of the plasma concentration ranges set forth herein.

The antiviral composition can be administered chronically, i.e. on a recurring basis, such as daily, every other day, every second day, every third day, every fourth day, every fifth day, every sixth day, weekly, every other week, every second week, every third week, monthly, bimonthly, semi-monthly, every other month every second month, quarterly, every other quarter, trimesterly, seasonally, semi-annually and/or annually. The treatment period one or more weeks, one or more months, one or more quarters and/or one or more years. An effective dose of cardiac glycoside (cardiac glycoside-containing composition) is administered one or more times in a day.

In some embodiments, the animal is administered 140 microg to 315 microg per day of cardiac glycoside. In some embodiments, a dose comprises 20 microg to 750 microg, 12 microg to 300 microg, or 12 microg to 120 microg of cardiac glycoside. The daily dose of cardiac glycoside can range from 20 microg to 750 microg, 0.01 microg to 100 mg, or 0.01 microg to 100 microg of cardiac glycoside/day.

The dose of cardiac glycoside can be also about 0.5 to about 500 microg/day or less, about 0.5 to about 400 microg/day or less, about 0.5 to about 300 microg/day or less, about 0.5 to about 200 microg/day or less, about 0.5 to about 100 microg/day or less, about 1 to about 80 microg/day, about 1.5 to about 60 microg/day, about 1.8 to about 60 microg/day, about 1.8 to about 40 microg/day.

In some embodiments, the cardiac glycoside is administered in at least two dosing phases: a loading phase and a maintenance phase. The loading phase is continued until about achievement of steady state plasma level of cardiac glycoside. The maintenance phase begins at either the initiation of therapy or after about completion of the loading phase. Dose titration can occur in the loading phase and/or the maintenance phase.

All dosing regimens, dosing schedules, and doses described herein are contemplated as being suitable; however, some dosing regimens, dosing schedules, and doses may be more suitable for some subject than for others. The target clinical endpoints are used to guide said dosing.

The composition can be administered systemically. Modes of systemic administration include parenteral, buccal, enteral, intramuscular, subdermal, sublingual, peroral, pulmonary, or oral. The composition can also be administered via injection or intravenously. The composition may also be administered by two or more routes to the same subject. In some embodiments, the composition is administered by a combination of any two or more modes of administration selected from the group consisting of parenteral, buccal, enteral, intramuscular, subdermal, sublingual, peroral, pulmonary, and oral.

The cardiac glycoside may also be included in a feed and/or a liquid and administered orally to the animal. The solid feed may comprise cardiac glycoside and at least one feedstuff. The liquid feed may comprise cardiac glycoside, at least one liquid, and at least one nutrient. Cardiac glycoside may also be administered in a milk substitute product or in water. The cardiac glycoside may also be administered to the animal by feeding the animal leaf material from the Nerium sp. plant. The leaf material may be dried or undried. In some embodiments, the antiviral composition excludes Nerium sp. or Digitalis lanata plant material.

The invention also provides a sublingual dosage form comprising oleandrin (or digoxin) and liquid carrier. The invention also provides a method of treating viral infection comprising sublingually administering plural doses of an oleandrin-containing (digoxin-containing) composition to an animal having said viral infection. One or more doses can be administered per day for two or more days per week and for one or more weeks per month, optionally for one or months per year. The liquid carrier can comprise water, oil, liquid feed, or a combination of any thereof.

In some embodiments, the antiviral composition comprises oleandrin (or digoxin or a combination of oleandrin and digoxin) and oil. The oil can comprise medium chain triglycerides (MCT). The antiviral composition can comprise one, two or more oleandrin-containing extracts and one or more pharmaceutical excipients.

In some embodiments, the glycoside-containing composition comprises an extract of Nerium sp., said extract comprising a) at least oleandrin; b) at least oleandrin, oleanolic acid, ursolic acid, and betulinic acid; or c) at least oleandrin, oleanolic acid, ursolic acid, betulinic acid, kanerocin, kanerodione, oleandrigenin, Nerium F, neritaloside, odoroside, adynerin, odoroside-G-acetate, and gitoxigenin.

The cardiac glycoside-containing composition (or the extract) may further comprise polyphenol(s), carbohydrate(s), flavonoid(s), amino acid(s), soluble protein(s), cellulose, starch, alkaloid(s), saponin(s), tannin(s), and any combination thereof.

The amino acid can be selected from the group consisting of aspartic acid, glutamic acid, asparagine, serine, glutamine, glycine, histidine, arginine, threonine, alanine, proline, tyrosine, valine, methionine, cysteine, isoleucine, leucine, phenylalanine, tryptophan, and lysine. In some embodiments, the amino is selected from the group consisting of asparagine, arginine, threonine, alanine, proline, tyrosine, valine, isoleucine, leucine, phenylalanine, tryptophan, and lysine.

If present in the antiviral composition, additional cardiac glycoside can be further included: odoroside or neritaloside. The aglycone oleandrigenin can also be further included. In some embodiments, the composition further comprises a) one or more triterpenes; b) one or more steroids; c) one or more triterpene derivatives; d) one or more steroid derivatives; or e) a combination thereof. In some embodiments, the composition comprises cardiac glycoside and a) two or three triterpenes; b) two or three triterpene derivatives; c) two or three triterpene salts; or d) a combination thereof. In some embodiments, the triterpene is selected from the group consisting of oleanolic acid, ursolic acid, betulinic acid, and salts or derivatives thereof.

Some embodiments of the invention include those wherein a pharmaceutical composition comprises at least one pharmaceutical excipient and the antiviral composition. In some embodiments, the antiviral composition comprises a) at least one cardiac glycoside and at least one triterpene; b) at least one cardiac glycoside and at least two triterpenes; c) at least one cardiac glycoside and at least three triterpenes; d) at least two triterpenes and excludes cardiac glycoside; e) at least three triterpenes and excludes cardiac glycoside; or f) at least one cardiac glycoside, e.g. oleandrin, digoxin. As used herein, the generic terms triterpene and cardiac glycoside also encompass salts and derivatives thereof, unless otherwise specified.

The cardiac glycoside can be present in a pharmaceutical composition in pure form or as part of an extract containing one or more cardiac glycosides. The triterpene(s) can be present in a pharmaceutical composition in pure form or as part of an extract containing triterpene(s). In some embodiments, the cardiac glycoside is present as the primary therapeutic component, meaning the component primarily responsible for antiviral activity, in the pharmaceutical composition.

In some embodiments, an oleandrin-containing extract is obtained by extraction of plant material. The extract can comprise a hot-water extract, cold-water extract, supercritical fluid (SCF) extract, subcritical liquid extract, organic solvent extract, or combination thereof of the plant material. In some embodiments, the extract has been (biomass) prepared by subcritical liquid extraction of Nerium plant mass (biomass) using, as the extraction fluid, subcritical liquid carbon dioxide, optionally comprising alcohol. In some embodiments, the oleandrin-containing composition comprises two or more different types of oleandrin-containing extracts.

Embodiments of the invention include those wherein the oleandrin-containing biomass (plant material) is Nerium sp., e.g. Nerium oleander, Nerium oleander L (Apocynaceae), Nerium odourum, Nerium indicum Mill, white oleander, pink oleander, Agrobacterium tumefaciens, cell culture (cellular mass) of any of said species, or a combination thereof. In some embodiments, the biomass comprises leaves, stems, flowers, bark, fruits, seeds, sap, and/or pods.

In some embodiments, the extract comprises at least one other pharmacologically active agent, obtained along with the cardiac glycoside during extraction, that contributes to the therapeutic efficacy of the cardiac glycoside when the extract is administered to an animal. In some embodiments, the composition further comprises one or more other non-cardiac glycoside therapeutically effective agents, i.e. one or more agents that are not cardiac glycosides. In some embodiments, the composition further comprises one or more antiviral compound(s). In some embodiments, the antiviral composition excludes a pharmacologically active polysaccharide.

For each embodiment of the invention, the preferred cardiac glycoside is a) oleandrin, b) digoxin, or c) a combination of oleandrin and digoxin.

In some embodiments, the extract comprises one or more cardiac glycosides and one or more cardiac glycoside precursors (such as cardenolides, cardadienolides and cardatrienolides, all of which are the aglycone constituents of cardiac glycosides, for example, digitoxin, acetyl digitoxin, digitoxigenin, digoxin, acetyl digoxin, digoxigenin, medigoxin, strophanthins, cymarine, ouabain, or strophanthidin). The extract may further comprise one or more glycone constituents of cardiac glycosides (such as glucoside, fructoside, and/or glucuronide) as cardiac glycoside precursors. Accordingly, the antiviral composition may comprise one or more cardiac glycosides and two more cardiac glycoside precursors selected from the group consisting of one or more aglycone constituents, and one or more glycone constituents. The extract may also comprise one or more other non-cardiac glycoside therapeutically effective agents obtained from Nerium sp. plant material.

In some embodiments, a composition containing oleandrin (OL), oleanolic acid (OA), ursolic acid (UA) and betulinic acid (BA) is more efficacious than pure oleandrin, when equivalent doses based upon oleandrin content are compared.

In some embodiments, the molar ratio of total triterpene content (OA+UA+BA) to oleandrin ranges from about 15:1 to about 5:1, or about 12:1 to about 8:1, or about 100:1 to about 15:1, or about 100:1 to about 50:1, or about 100:1 to about 75:1, or about 100:1 to about 80:1, or about 100:1 to about 90:1, or about 10:1.

In some embodiments, the molar ratios of the individual triterpenes to oleandrin range as follows: about 2-8 (OA):about 2-8 (UA):about 0.1-1 (BA):about 0.5-1.5 (OL); or about 3-6 (OA):about 3-6 (UA):about 0.3-8 (BA):about 0.7-1.2 (OL); or about 4-5 (OA):about 4-5 (UA):about 0.4-0.7 (BA):about 0.9-1.1 (OL); or about 4.6 (OA):about 4.4 (UA):about 0.6 (BA):about 1 (OL).

In some embodiments, the other therapeutic agent, such as that obtained by extraction of Nerium sp. plant material, is not a polysaccharide obtained during preparation of the extract, meaning it is not an acidic homopolygalacturonan or arabinogalaturonan. In some embodiments, the extract excludes another therapeutic agent and/or excludes an acidic homopolygalacturonan or arabinogalaturonan obtained during preparation of the extract.

In some embodiments, the other therapeutic agent, such as that obtained by extraction of Nerium sp. plant material, is a polysaccharide obtained during preparation of the extract, e.g. an acidic homopolygalacturonan or arabinogalaturonan. In some embodiments, the extract comprises another therapeutic agent and/or comprises an acidic homopolygalacturonan or arabinogalaturonan obtained during preparation of the extract from said plant material.

In some embodiments, the extract comprises oleandrin and at least one other compound selected from the group consisting of cardiac glycoside, glycone, aglycone, steroid, triterpene, polysaccharide, saccharide, alkaloid, fat, protein, neritaloside, odoroside, oleanolic acid, ursolic acid, betulinic acid, oleandrigenin, oleaside A, betulin (urs-12-ene-3β,28-diol), 28-norurs-12-en-3β-ol, urs-12-en-3β-ol, 3β,3β-hydroxy-12-oleanen-28-oic acid, 3β,20α-dihydroxyurs-21-en-28-oic acid, 3β,27-dihydroxy-12-ursen-28-oic acid, 3β,13β-dihydroxyurs-11-en-28-oic acid, 3β,12α-dihydroxyoleanan-28,13β-olide, 3β,27-dihydroxy-12-oleanan-28-oic acid, homopolygalacturonan, arabinogalaturonan, chlorogenic acid, caffeic acid, L-quinic acid, 4-coumaroyl-CoA, 3-O-caffeoylquinic acid, 5-O-caffeoylquinic acid, cardenolide B-1, cardenolide B-2, oleagenin, neridiginoside, nerizoside, odoroside-H, 3-beta-O-(D-diginosyl)-5-beta, 14 beta-dihydroxy-card-20(22)-enolide pectic polysaccharide composed of galacturonic acid, rhamnose, arabinose, xylose, and galactose, polysaccharide with MW in the range of 17000-120000 D, or MW about 35000 D, about 3000 D, about 5500 D, or about 12000 D, cardenolide monoglycoside, cardenolide N-1, cardenolide N-2, cardenolide N-3, cardenolide N-4, pregnane, 4,6-diene-3,12,20-trione, 20R-hydroxypregna-4,6-diene-3,12-dione, 16beta,17beta-epoxy-12beta-hydroxypregna-4,6-diene-3,20-dione, 12beta-hydroxypregna-4,6,16-triene-3,20-dione (neridienone A), 20S,21-dihydroxypregna-4,6-diene-3,12-dione (neridienone B), neriucoumaric acid, isoneriucoumaric acid, oleanderoic acid, oleanderen, 8alpha-methoxylabdan-18-oic acid, 12-ursene, kaneroside, neriumoside, 3β-O-(D-diginosyl)-2α-hydroxy-8,14β-epoxy-5β-carda-16:17, 20:22-dienolide, 3β-O-(D-diginosyl)-2α,14β-dihydroxy-5β-carda-16:17,20:22-dienolide, 3β,27-dihydroxy-urs-18-en-13,28-olide, 3β,22α,28-trihydroxy-25-nor-lup-1(10),20(29)-dien-2-one, cis-karenin (3β-hydroxy-28-Z-p-coumaroyloxy-urs-12-en-27-oic acid), trans-karenin (3-β-hydroxy-28-E-p-coumaroyloxy-urs-12-en-27-oic acid), 3beta-hydroxy-5alpha-carda-14(15),20(22)-dienolide (beta-anhydroepidigitoxigenin), 3 beta-O-(D-digitalosyl)-21-hydroxy-5beta-carda-8,14,16,20(22)-tetraenolide (neriumogenin-A-3beta-D-digitaloside), proceragenin, neridienone A, 3beta,27-dihydroxy-12-ursen-28-oic acid, 3beta,13beta-dihydroxyurs-1 1-en-28-oic acid, 3beta-hydroxyurs-12-en-28-aldehyde, 28-orurs-12-en-3beta-ol, urs-12-en-3beta-ol, urs-12-ene-3beta,28-diol, 3beta,27-dihydroxy-12-oleanen-28-oic acid, (20S,24R)-epoxydammarane-3beta,25-diol, 20beta,28-epoxy-28alpha-methoxytaraxasteran-3beta-ol, 20beta,28-epoxytaraxaster-21-en-3beta-ol, 28-nor-urs-12-ene-3beta,17 beta-diol, 3beta-hydroxyurs-12-en-28-aldehyde, alpha-neriursate, beta-neriursate, 3alpha-acetophenoxy-urs-12-en-28-oic acid, 3beta-acetophenoxy-urs-12-en-28-oic acid, oleanderolic acid, kanerodione, 3β-p-hydroxyphenoxy-11α-methoxy-12α-hydroxy-20-ursen-28-oic acid, 28-hydroxy-20(29)-lupen-3,7-dione, kanerocin, 3alpha-hydroxy-urs-18,20-dien-28-oic acid, D-sarmentose, D-diginose, neridiginoside, nerizoside, isoricinoleic acid, gentiobiosylnerigoside, gentiobiosylbeaumontoside, gentiobiosyloleandrin, folinerin, 12β-hydroxy-5β-carda-8,14,16,20(22)-tetraenolide, 8μ-hydroxy-digitoxigenin, Δ16-8β-hydroxy-digitoxigenin, Δ16-neriagenin, uvaol, ursolic aldehyde, 27(p-coumaroyloxy)ursolic acid, oleanderol, 16-anhydro-deacteyl-nerigoside, 9-D-hydroxy-cis-12-octadecanoic acid, adigoside, adynerin, alpha-amyrin, beta-sitosterol, campestrol, caoutchouc, capric acid, caprylic acid, choline, cornerin, cortenerin, deacetyloleandrin, diacetyl-nerigoside, foliandrin, pseudocuramine, quercetin, quercetin-3-rhamnoglucoside, quercitrin, rosaginin, rutin, stearic acid, stigmasterol, strospeside, urehitoxin, and uzarigenin. Additional components that may be present in the extract are disclosed by Gupta et al. (IJPSR (2010(, 1(3), 21-27, the entire disclosure of which is hereby incorporated by reference).

Oleandrin may also be obtained from extracts of suspension cultures derived from Agrobacterium tumefaciens-transformed calli. Hot water, organic solvent, aqueous organic solvent, subcritical liquid extract, or supercritical fluid extract of agrobacterium may be used according to the invention.

Oleandrin may also be obtained from extracts of Nerium sp. microculture in vitro, whereby shoot cultures can be initiated from seedlings and/or from shoot apices of the Nerium sp. cultivars, e.g. Splendens Giganteum, Revanche or Alsace, or other cultivars. Hot water, organic solvent, aqueous organic solvent, or supercritical fluid extracts of microcultured Nerium sp. may be used according to the invention.

The extract may also be obtained by extraction of cellular mass (such as is present in cell culture) of any of said plant species.

The invention also provides use of a cardiac glycoside in the manufacture of a medicament for the treatment of viral infection in an animal. In some embodiments, the manufacture of such a medicament comprises: providing one or more antiviral compounds of the invention; including a dose of antiviral compound(s) in a pharmaceutical dosage form; and packaging the pharmaceutical dosage form. In some embodiments, the manufacture can be conducted as described in PCT International Application No. PCT/US06/29061. The manufacture can also include one or more additional steps such as: delivering the packaged dosage form to a vendor (retailer, wholesaler and/or distributor); selling or otherwise providing the packaged dosage form to an animal having a viral infection; including with the medicament a label and a package insert, which provides instructions on use, dosing regimen, administration, content and toxicology profile of the dosage form. In some embodiments, the treatment of viral infection comprises: determining that an animal has a viral infection; indicating administration of pharmaceutical dosage form to the animal according to a dosing regimen; administering to the animal one or more pharmaceutical dosage forms, wherein the one or more pharmaceutical dosage forms is administered according to the dosing regimen.

The pharmaceutical composition can further comprise a combination of at least one material selected from the group consisting of a water soluble (miscible) co-solvent, a water insoluble (immiscible) co-solvent, a surfactant, an antioxidant, a chelating agent, and an absorption enhancer.

The solubilizer is at least a single surfactant, but it can also be a combination of materials such as a combination of: a) surfactant and water miscible solvent; b) surfactant and water immiscible solvent; c) surfactant, antioxidant; d) surfactant, antioxidant, and water miscible solvent; e) surfactant, antioxidant, and water immiscible solvent; f) surfactant, water miscible solvent, and water immiscible solvent; or g) surfactant, antioxidant, water miscible solvent, and water immiscible solvent.

The pharmaceutical composition optionally further comprises a) at least one liquid carrier; b) at least one emulsifying agent; c) at least one solubilizing agent; d) at least one dispersing agent; e) at least one other excipient; or f) a combination thereof.

In some embodiments, the water miscible solvent is low molecular weight (less than 6000) PEG, glycol, or alcohol. In some embodiments, the surfactant is a pegylated surfactant, meaning a surfactant comprising a poly(ethylene glycol) functional group.

The invention includes all combinations of the aspects, embodiments and sub-embodiments of the invention disclosed herein.

### BRIEF DESCRIPTION OF THE FIGURES

The following figures form part of the present description and describe exemplary embodiments of the claimed invention. The skilled artisan will, in light of these figures and the description herein, be able to practice the invention without undue experimentation.

FIGS. 1A and 1B depict charts summarizing the in vitro dose response therapeutic antiviral activity of oleandrin (FIG. 1A) and extract containing oleandrin (FIG. 1B; PBI-oleandrin) as compared to control (DMSO vehicle) against bovine coronavirus as determined in HRT cells. (Example 6)

FIGS. 2A and 2B depict charts summarizing the in vitro dose response prophylactic antiviral activity of oleandrin (FIG. 2A) and extract containing oleandrin (FIG. 2B: PBI-oleandrin) against bovine coronavirus as determined in HRT cells. (Example 22)

FIGS. 3A and 3B depict charts summarizing the in vitro dose response therapeutic antiviral activity of oleandrin (FIG. 3A) and extract containing oleandrin (FIG. 3B; PBI-oleandrin) as compared to control (DMSO vehicle) against BVDV as determined in MDBK cells. (Example 13)

FIGS. 4A and 4B depict charts summarizing the in vitro dose response prophylactic antiviral activity of oleandrin (FIG. 4A) and extract containing oleandrin (FIG. 4B: PBI-oleandrin) against BVDV as determined in MDBK cells. (Example 22)

FIGS. 5A and 5B depict charts summarizing the in vitro dose response therapeutic antiviral activity of oleandrin (FIG. 5A) and extract containing oleandrin (FIG. 5B; PBI-oleandrin) as compared to control (DMSO vehicle) against PRRSV as determined in MARC 145 cells. (Example 14)

FIGS. 6A and 6B depict charts summarizing the in vitro dose response prophylactic antiviral activity of oleandrin (FIG. 6A) and extract containing oleandrin (FIG. 6B: PBI-oleandrin) against PRRSV as determined in MARC 145 cells. (Example 23)

FIGS. 7A and 7B depict charts summarizing the in vitro dose response therapeutic antiviral activity of oleandrin (FIG. 7A) and extract containing oleandrin (FIG. 7B; PBI-oleandrin) as compared to control (DMSO vehicle) against BRSV as determined in BT cells. (Example 15)

FIGS. 8A and 8B depict charts summarizing the in vitro dose response prophylactic antiviral activity of oleandrin (FIG. 8A) and extract containing oleandrin (FIG. 8B: PBI-oleandrin) against BRSV as determined in BT 145 cells. (Example 24)

### DETAILED DESCRIPTION OF THE INVENTION

The invention provides a method of treating viral infection in an animal by chronic or acute administration of one or more effective doses of antiviral composition (or pharmaceutical composition comprising the antiviral composition and at least one pharmaceutical excipient) to the animal. The composition is administered according to a dosing regimen best suited for the animal, the suitability of the dose and dosing regimen to be determined clinically according to conventional clinical practices and clinical treatment endpoints for viral infection.

As used herein, the term “subject” is taken to mean warm blooded animals such as birds and mammals, for example, pig, cow, horse, sheep, goat, llama, alpaca, buffalo, deer, elk, giraffe, camel, dog, cat, chicken, turkey, pigeon, duck, pheasant, guinea, or other animal. Livestock animals are particularly suitable as subjects.

An animal treated according to the invention will exhibit a therapeutic response. By “therapeutic response” is meant that an animal suffering from the viral infection will enjoy at least one of the following clinical benefits as a result of treatment with a cardiac glycoside: reduction of the active viral titer in the animal's blood or plasma, eradication of active virus from the animal's blood or plasma, amelioration of the infection, reduction in the occurrence of symptoms associated with the infection, partial or full remission of the infection or increased time to progression of the infection, and/or reduction in the infectivity of the virus causing said viral infection. The therapeutic response can be a full or partial therapeutic response.

As used herein, “time to progression” is the period, length or duration of time after viral infection is diagnosed (or treated) until the infection begins to worsen. It is the period of time during which the level of infection is maintained without further progression of the infection, and the period of time ends when the infection begins to progress again. Progression of a disease is determined by “staging” an animal suffering from the infection prior to or at initiation of therapy. For example, the animal's health is determined prior to or at initiation of therapy. The animal is then treated with antiviral composition, and the viral titer is monitored periodically. At some later point in time, the symptoms of the infection may worsen, thus marking progression of the infection and the end of the “time to progression”. The period of time during which the infection did not progress or during which the level or severity of the infection did not worsen is the “time to progression”.

A dosing regimen includes a therapeutically relevant dose (or effective dose) of one or more cardiac glycosides, and/or triterpene(s), administered according to a dosing schedule. A therapeutically relevant dose, therefore, is a therapeutic dose at which a therapeutic response of the viral infection to treatment with antiviral composition is observed and at which an animal can be administered the antiviral composition without an excessive amount of unwanted or deleterious side effects. A therapeutically relevant dose is non-lethal to an animal, even though it may cause some side effects in the animal. It is a dose at which the level of clinical benefit to an animal being administered the antiviral composition exceeds the level of deleterious side effects experienced by the animal due to administration of the antiviral composition or component(s) thereof.

A therapeutically relevant dose will vary from animal to animal according to a variety of established pharmacologic, pharmacodynamic and pharmacokinetic principles. However, a therapeutically relevant dose (relative, for example, to oleandrin) can be about 25 micrograms, about 100 micrograms, about 250 micrograms, about 500 micrograms or about 750 micrograms of cardiac glycoside/day or it can be in the range of about 25-750 micrograms of cardiac glycoside per dose, or might not exceed about 25 micrograms, about 100 micrograms, about 250 micrograms, about 500 micrograms or about 750 micrograms of cardiac glycoside/day. Another example of a therapeutically relevant dose (relative, for example, to triterpene either individually or together) will typically be in the range of about 0.1 micrograms to 100 micrograms, about 0.1 microg to about 500 microg, about 1 to about 100 microg per kg of body weight, about 15 to about 25 microg/kg, about 25 to about 50 microg/kg, about 50 to about 100 microg/kg, about 100 to about 200 microg/kg, about 200 to about 500 microg/kg, about 10 to about 750 microg/kg, about 16 to about 640 microg/kg, about 15 to about 750 microg/kg, about 15 to about 700 microg/kg, or about 15 to about 650 microg/kg of body weight.

It is known in the art that the actual amount of antiviral composition required to provide a target therapeutic result in an animal may vary from subject to subject according to the basic principles of pharmacy.

Oleandrin may be administered to ruminant animals including ruminants include cattle, sheep, goats, buffalo, deer, elk, giraffes, and camels.

For ruminant animals, the young animals have a different digestive tract than adult animals. Accordingly, the dose of oleandrin (microg of oleandrin per Kg of bodyweight) may be different in a young animal as compared to an adult animal of the same species. For example, a calf may require a different dose than a cow in order to benefit from oleandrin therapy. A veterinary clinician will be able to use known dose escalation or de-escalation protocols to determine a safe and effective dose to be administered.

A therapeutically relevant dose can be administered according to any dosing regimen typically used in the treatment of viral infection. A therapeutically relevant dose can be administered once, twice, thrice, or more, or continuously daily. It can be administered every other day, every third day, every fourth day, every fifth day, semiweekly, weekly, biweekly, every three weeks, every four weeks, monthly, bimonthly, semimonthly, every three months, every four months, semiannually, annually, or according to a combination of any of the above to arrive at a suitable dosing schedule. For example, a therapeutically relevant dose can be administered one or more times daily (up to 10 times daily for the highest dose) for one or more weeks.

Oleandrin may be included in feed and/or liquid administered to an animal. Oleandrin may be include in any feed format including solid feed, liquid feed, or gel feed. The solid feed may be loose granules, pellets, foodstuff, block or other such feed used to feed animals.

The solid feed may comprise oleandrin and at least one feedstuff. Suitable feedstuffs include Whole cottonseed, cottonseed hulls, cottonseed meal, soybean meal, soybean hulls, corn gluten feed, hominy feed, dried distiller's grains, and rice mill feed are examples of commodity feedstuffs. Additional ingredients that may be included are selected from the group consisting of silage, nutritious supplement, vitamin, mineral, salt, grain (wheat, barley, oat, corn), fiber, hay, alfalfa, rye grass, beet, molasses, blood meal, bone meal, yeast, brome grass, canary grass, tomato, carrot, peas, pea vine hay, safflower, sage brush, sorghum, cheatgrass, clover, fat, grape, hominy, hops, meadow hay, sundan grass, sunflower, timothy hay, meat meal, milo, orange, orchard grass, potato, navy beans, peanut, prairie hay, rape meal, soybean, protein, others, and combinations of any thereof.

The liquid feed comprises oleandrin, at least one liquid, and at least one nutrient. The liquid can be water, fermentation broth, milk, or milk substitute or other such liquid suitable for administration to an animal.

Given the bitter taste of oleandrin and oleander extracts, an oral composition administered to an animal can include one or more taste-masking agents. A sweetener, e.g. molasses, is advantageously included in a feed.

Oleandrin can also be included in water or other liquid given to the animal.

A composition can also include one or more additives suitable for administration to animals. For example, ammonium sulfate, calcium carbonate, sodium chloride, defluorinated phosphate, diammonium phosphate, dicalcium phosphate, limestone, monoammonium phosphate, monocalcium phosphate, sodium tripolyphos, urea, or any combination thereof may be used as additive.

The invention provides a method of treating viral infection in a mammal or host cell, the method comprising: administering an antiviral composition to the mammal or host cell prior to contraction of said viral infection, whereby upon viral infection of said mammal or host cell, the antiviral composition reduces the viral titer and ameliorates or eliminates the viral infection.

The antiviral composition of the invention: a) can be administered prophylactically before viral infection to inhibit viral infection after exposure to virus; b) can be administered after viral infection to inhibit or reduce viral replication and production of infectious progeny; or c) a combination of a) and b).

The invention provides a method of treating a viral infection, caused by a virus of the Arterviridae, Flaviviridae, Paramyxoviridae, Picornaviridae, Chordopoxvirinae, Poxviridae, Coronaviridae, Papillomaviridae, Rhabdoviridae, Parvoviridae, Orthomyxoviridae, Reoviridae, Astroviridae, or Circoviridae family, in an animal or host cell, the method comprising administering an effective amount of the antiviral composition, thereby exposing the virus to the antiviral composition and treating said viral infection.

Antiviral activity of the compositions herein was evaluated against rhinovirus infection. Rhinovirus is of the Picomaviridae family and Enterovirus genus. It is not enveloped and is an ss-RNA virus of (+) polarity. Oleandrin was found to be inactive against rhinovirus in the concentrations and assays employed herein, because it did not inhibit viral replication. Oleandrin was also found to be inactive against Human adenovirus (HAdv-C5; Adenoviridae, Mastadenovirus), dengue fever virus (Flaviviridae, flavivirus), Omsk hemorrhagic fever virus (Flaviviridae, flavivirus), Kyasanur forest disease virus (Flaviviridae, flavivirus), and Alkhuma hemorrhagic fever virus (Flaviviridae, flavivirus). Moreover, oleandrin has been reported to be inactive against murine coronavirus.

The antiviral activity, both therapeutic and prophylactic, of oleandrin and extract containing oleandrin was established by in vitro assays in accepted cell culture assays.

Proof of the efficacy of oleandrin (oleandrin-containing composition) against bovine coronavirus (BCV), was obtained through in vitro evaluation according to Example 6, wherein HRT cells infected with BCV were treated with oleandrin. HRT cells were plated 48 hours prior to the assay. At the time of the assay, the media was removed and replaced with virus maintenance media containing BCV at an MOI of 0.01 in each well. A separate set of plates was incubated for either 12 or 24 hours. At each time point (12 or 24 hours), plates were washed gently 3 times with 1×DPBS and then 2 ml of virus maintenance medium containing the desired concentrations of Oleandrin or PBI-05204 dissolved in DMSO, or matched concentrations of DMSO-only was added to each well. Oleandrin, PBI, and DMSO dilutions were made and stored protected from light at 4° C. Samples were removed at 24 and 48 hours after the 12-hour virus inoculation, and at 48 hours after the 24-hour virus inoculation and aliquoted into two cryovials. Virus isolations were performed immediately on samples collected at each time point and the aliquots were then frozen at −80° C. Samples were submitted for qRT-qPCR analysis.

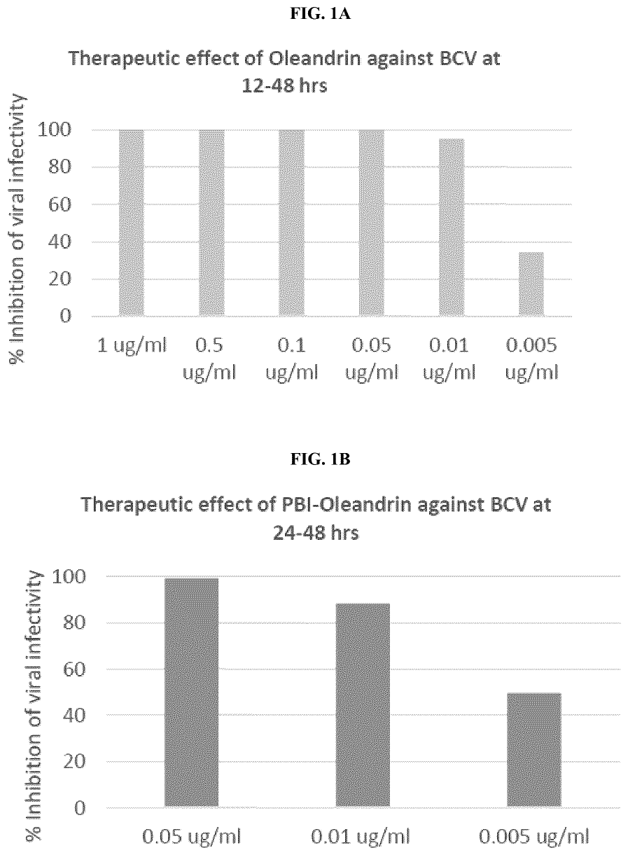

The results in FIG. 1A (oleandrin as sole active) and FIG. 1B (oleander extract containing oleandrin) indicate that a) oleandrin caused a 98-100% reduction in viral infectivity at the 24-h time and a similar 99-100% reduction at the 48-h time point; b) oleandrin is efficacious over the entire concentration range of about 0.01 microg/ml and higher; c) oleandrin should be administered repeatedly, since a single dose is not sufficient to fully stop viral replication; and d) oleandrin is very effective at inhibiting infectivity of progeny virus. The results also indicated that oleandrin at concentrations of up to 1.0 microg/mL is not toxic to HRT cells.

Accordingly, the invention provides a method of treating bovine coronavirus infection, the method comprising administering a therapeutically effective amount of oleandrin to an animal having said infection.

The prophylactic efficacy of oleandrin and an oleandrin-containing extract against BCV was evaluated according to Example 22. HRT cells were plated in 12 well-plates 48 hours prior to the assay. At the time of the assay, the media was removed from each well and replaced with 200p0 of media containing the desired concentrations of Oleandrin or PBI-extract in DMSO, or matched concentrations of DMSO-only. Oleandrin, PBI, and DMSO dilutions were made fresh prior to the assay. Plates were incubated with the products for 30 minutes, then BCV at MOI of 0.01 was added to each well (1×10⁴ TCID50 per well) in 500 μL of virus maintenance media. Virus was incubated on the plates for 1 hour and then removed. Plates were washed gently 3 times with 1×DPBS followed by adding 2 ml of virus maintenance medium containing Oleandrin, PBI-extract, or DMSO-only to each well. Samples were removed at each time point (24 and 48 hrs) and aliquoted into two cryovials. One aliquot was used for virus isolation and the second one was used for RT-qPCR. Virus isolation was performed immediately on samples collected at each time point and the aliquots were then frozen at −80° C. Samples were submitted for RT-qPCR analysis.

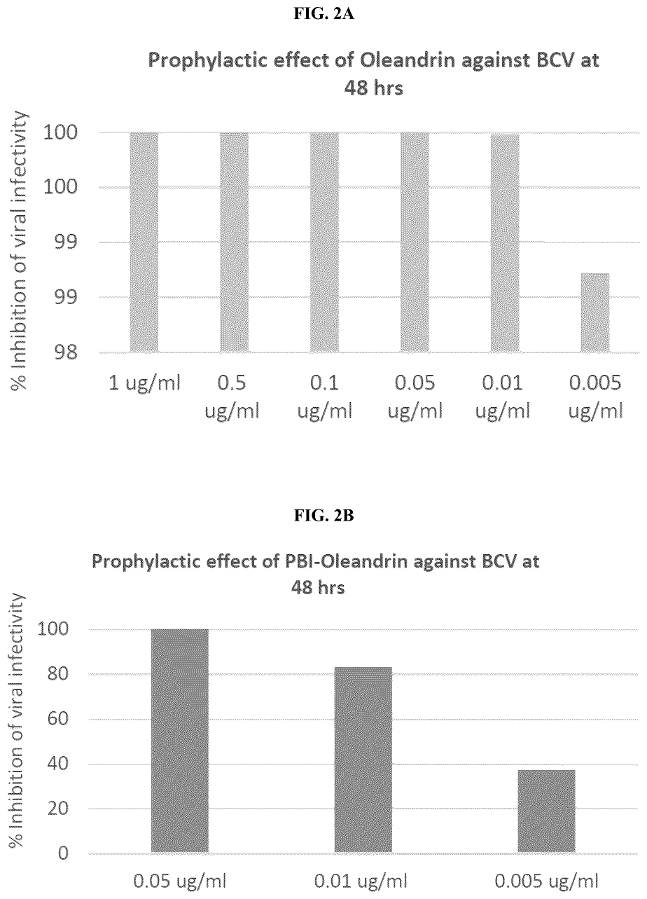

The results in FIG. 2A (oleandrin as sole active) and FIG. 2B (oleander extract containing oleandrin) indicate that a 30 min preincubation of cells with oleandrin prior to cell infection with BCV resulted in 99%-100% inhibition of viral infectivity at oleandrin concentrations between 0.01 to 1 ug/ml.

Accordingly, the invention provides a method of preventing progression of bovine coronavirus infection to a disease state, the method comprising administering a therapeutically effective amount of oleandrin to an animal having said bovine coronavirus infection.

Proof of the efficacy of oleandrin (oleandrin-containing composition) against bovine viral diarrhea virus (BVDV), was obtained through in vitro evaluation according to Example 12, wherein MDBK cells infected with BVDV were treated with oleandrin. MDBK cells were plated 48 hours prior to the assay. At the time of the assay, the media was removed and replaced with virus maintenance media containing the virus at an MOI of 0.01 in each well. A separate set of plates was incubated for either 12 or 24 hours. At each time point (12 or 24 hours), plates were washed gently 3 times with 1×DPBS and then 2 ml of virus maintenance medium containing the desired concentrations of Oleandrin or PBI-05204 dissolved in DMSO, or matched concentrations of DMSO-only was added to each well. Oleandrin, PBI-extract, and DMSO dilutions were made and stored protected from light at 4° C. Samples were removed at 24 and 48 hours after the 12-hour virus inoculation, and 48 hours after the 24-hour virus inoculation and aliquoted into two cryovials. Virus isolations were performed immediately on samples collected at each time point and the aliquots were then frozen at −80° C. Samples were submitted for qRT-qPCR analysis.

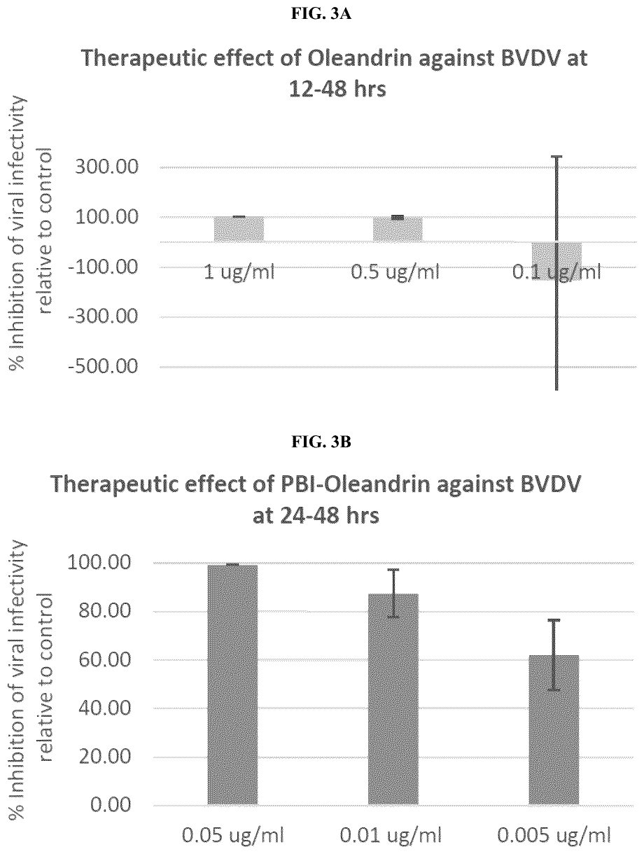

The results in FIG. 3A (oleandrin as sole active) and FIG. 3B (oleander extract containing oleandrin) indicate that a) oleandrin pretreatment caused a 91-94% inhibition of viral infectivity relative to control at the 24-h time point and a 98-100% reduction at the 48-h time point; b) oleandrin is efficacious over the entire concentration range of about 0.005 to 1.0 ug/ml; c) oleandrin should be administered repeatedly, since a single dose is not sufficient to fully stop viral replication; and d) oleandrin is effective at reducing viral infectivity of progeny virions. The results also indicated that oleandrin, at concentrations of up to 1.0 microg/mL, is not toxic to MDBK cells.

Accordingly, the invention provides a method of treating bovine viral diarrhea virus infection, the method comprising administering a therapeutically effective amount of oleandrin to an animal having said infection.

The prophylactic efficacy of oleandrin and an oleandrin-containing extract against BVDV was evaluated according to Example 23. MDBK cells were plated 48 hours prior to the assay. At the time of the assay, the media was removed from each well and replaced with 200 ul of media containing the desired concentrations of Oleandrin or PBI-05204 dissolved in DMSO, or matched concentrations of DMSO-only. Oleandrin, PBI-extract, and DMSO dilutions were made fresh prior to the assay. Plates were incubated with the products for 30 minutes, then BVDV virus at an MOI of 0.01 was added to each well. Virus was incubated on the plates for 1 hour and then removed. Plates were washed gently 3 times with 1×DPBS and 2 ml of virus maintenance medium containing Oleandrin, PBI-extract, or DMSO-only was added to each well. Samples were removed at each time point (24 and 48 hrs) and aliquoted into two cryovials. Virus isolations were performed immediately on samples collected at each time point and the aliquots were then frozen at −80° C. Samples were submitted for RT-qPCR analysis.

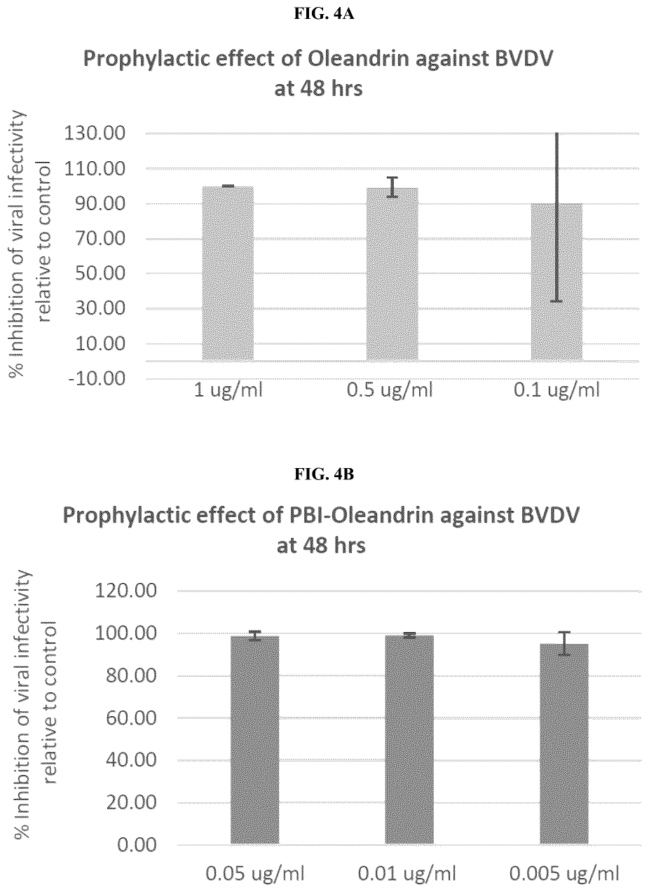

The results in FIG. 4A (oleandrin as sole active) and FIG. 4B (oleander extract containing oleandrin) indicate that a) a 30 min preincubation of cells with oleandrin prior to infection of cells with BVDV results in a 85-93% inhibition of infectivity of progeny cells when measured at 48 hr post infection of original parental cells when concentrations of 0.1 to 1.0 ug/ml oleandrin were used); and b) preincubation of cells with PBI-oleandrin extract 30 min prior to infection of cells with BVDV resulted in 95-100% inhibition of infectivity of the virus against progeny cells when concentrations of 0.005 to 0.05 ug/ml were used.

Accordingly, the invention provides a method of preventing progression of bovine viral diarrhea virus infection to a disease state, the method comprising administering a therapeutically effective amount of oleandrin to an animal having said infection.

Proof of the efficacy of oleandrin (oleandrin-containing composition) against porcine reproductive and respiratory syndrome virus (PRRSV), was obtained through in vitro evaluation according to Example 14, wherein MARC 145 cells infected with BVDV were treated with oleandrin pre- and post-infection. MARC 145 cells were plated 48 hours prior to the assay. At the time of the assay, the media was removed and replaced with virus maintenance media containing PRRSV at an MOI of 0.01 in each well. A separate set of plates was incubated for either 12 or 24 hours. At each timepoint (12 or 24 hours), plates were washed gently 3 times with 1×DPBS and then 2 ml of virus maintenance medium containing the desired concentrations of Oleandrin or PBI-05204 dissolved in DMSO, or matched concentrations of DMSO-only was added to each well. Oleandrin, PBI-extract, and DMSO dilutions were made and stored protected from light at 4° C. Samples were removed at 24 and 48 hours after the 12-hour virus inoculation, and at 48 hours after the 24-hour virus inoculation and aliquoted into two cryovials. Virus isolations were performed immediately on samples collected at each time point and the aliquots were then frozen at −80° C. Samples were submitted for qRT-qPCR analysis.

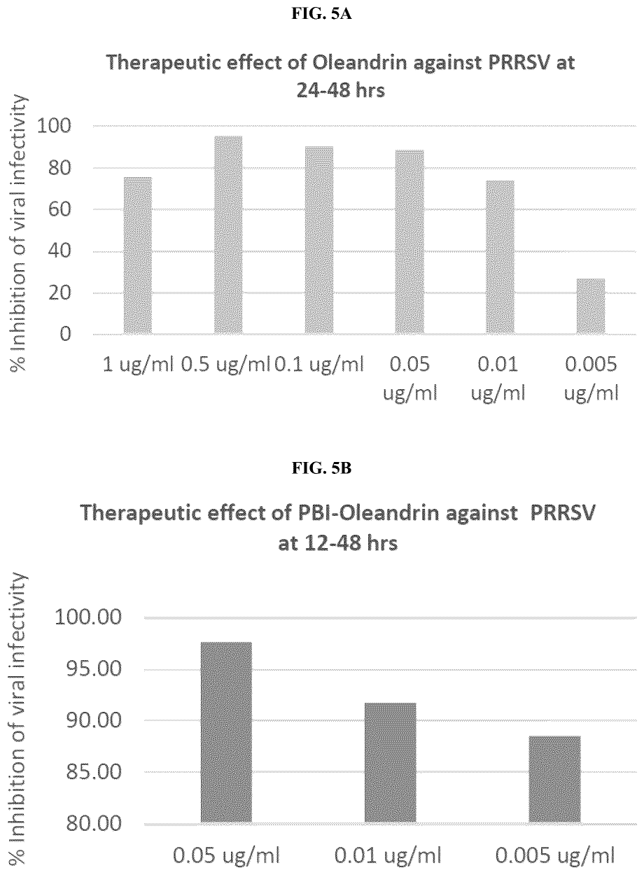

The results in FIG. 5A (oleandrin as sole active) and FIG. 5B (oleander extract containing oleandrin) indicate that a) oleandrin treatment caused a 78-98% inhibition of viral infectivity of progeny virus to new cells 24-48 h time period over the concentration range of 0.01 to 1 ug/ml oleandrin; b) oleandrin is efficacious over the entire concentration range of about 0.05 ug/ml and higher; c) oleandrin should be administered repeatedly, since a single dose is not sufficient to fully stop viral replication; and d) oleandrin is effective at reducing viral infectivity of progeny virions. The results also indicated that oleandrin, at concentrations of up to 1.0 microg/mL, is not toxic to MARC 145 cells.

Accordingly, the invention provides a method of treating PRRSV infection in an animal, the method comprising administering a therapeutically effective amount of oleandrin to said animal having said infection.

The prophylactic efficacy of oleandrin and an oleandrin-containing extract PRRSV was evaluated according to Example 24. MARC 145 cells were plated in 12 well-plates 48 hours prior to the assay. At the time the of the assay, the media was removed from each well and replaced with 200p0 of media containing the desired concentrations of Oleandrin or PBI-05204 dissolved in DMSO, or matched concentrations of DMSO-only. Oleandrin, PBI-extract, and DMSO dilutions were made fresh prior to the assay. Plates were incubated with product for 30 minutes, then PRRSV virus at MOI of 0.01 was added to each well (1×104 TCID50 per) in 500 μL of virus maintenance media. Virus was incubated on the plates for 1 hour and then removed. Plates were washed gently 3 times with 1×DPBS followed by adding 2 ml of virus maintenance medium containing Oleandrin, PBI-extract, or DMSO-only to each well. Samples were removed at each time point (24 and 48 hrs) and aliquoted into two cryovials. One aliquot was used for virus isolation and the second one was used for RT-qPCR. Virus isolation was performed immediately on samples collected at each time point and the aliquots were then frozen at −80° C. Samples were submitted for RT-qPCR analysis.

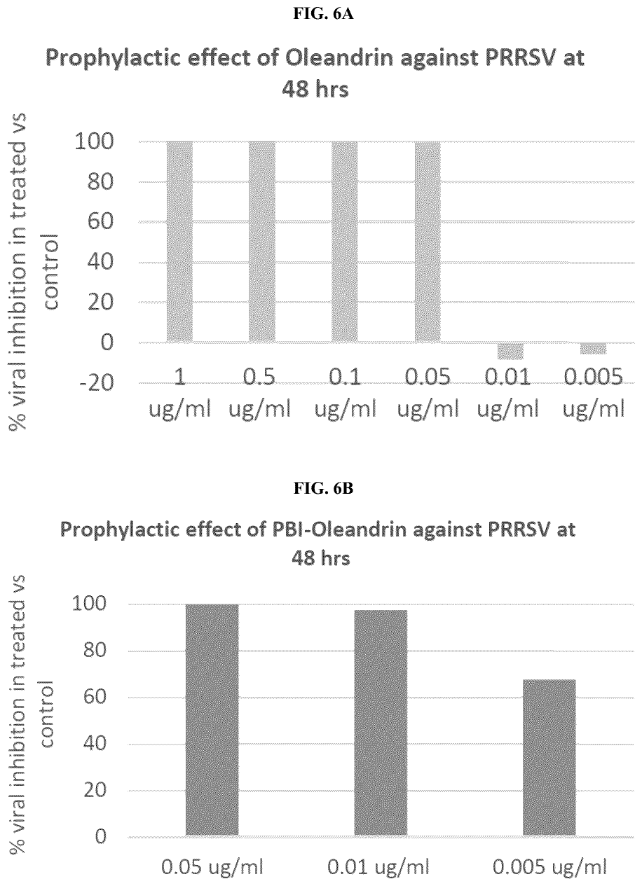

The results in FIG. 6A (oleandrin as sole active) and FIG. 6B (oleander extract containing oleandrin) indicate that a) a 30 min pretreatment of cells with oleandrin prior to infection of cells with PRRSV resulted in a 99% to 100% viral inhibition over the oleandrin concentration range of 0.05 to 1 microg/ml when measured at 48 hr post infection. The data in FIG. 6B demonstrate that a 30 min preincubation of cells with PBI-oleandrin prior to infection with PRRSV produced a 68 to 100% viral inhibition over the concentration range of 0.005 to 0.05 ug/ml when measured at 48 post virus infection.

Accordingly, the invention provides a method of preventing progression of PRRSV infection to a disease state, the method comprising administering a therapeutically effective amount of oleandrin to an animal having said infection.

Proof of the efficacy of oleandrin (oleandrin-containing composition) against bovine respiratory syncytial virus (BRSV), was obtained through in vitro evaluation according to Example 15, wherein BT cells infected with BVDV were treated with oleandrin. BT cells were plated 48 hours prior to the assay. At the time of the assay, the media was removed and replaced with virus maintenance media containing virus at an MOI of 0.01 in each well. A separate set of plates was incubated for either 12 or 24 hours. At each timepoint (12 or 24 hours), plates were washed gently with DPBS and then 2 ml of virus maintenance medium containing the desired concentrations of Oleandrin or PBI-05204 dissolved in DMSO, or matched concentrations of DMSO-only was added to each well. Oleandrin, PBI, and DMSO dilutions were made 5-hours prior to the 12-hour treatment and stored protected from light at 4° C. (prepared at 4 pm and used at 9 pm). Fresh Oleandrin, PBI, and DMSO dilutions were made prior to the 24-hour treatment. Samples were removed at 24 and 48 hours after the 12-hour virus inoculation, and at 48 hours after the 24-hour virus inoculation and aliquoted into two cryovials. Virus isolations were performed immediately on samples collected at each time point and the aliquots were then frozen at −80° C. Samples were submitted for qRT-qPCR analysis.

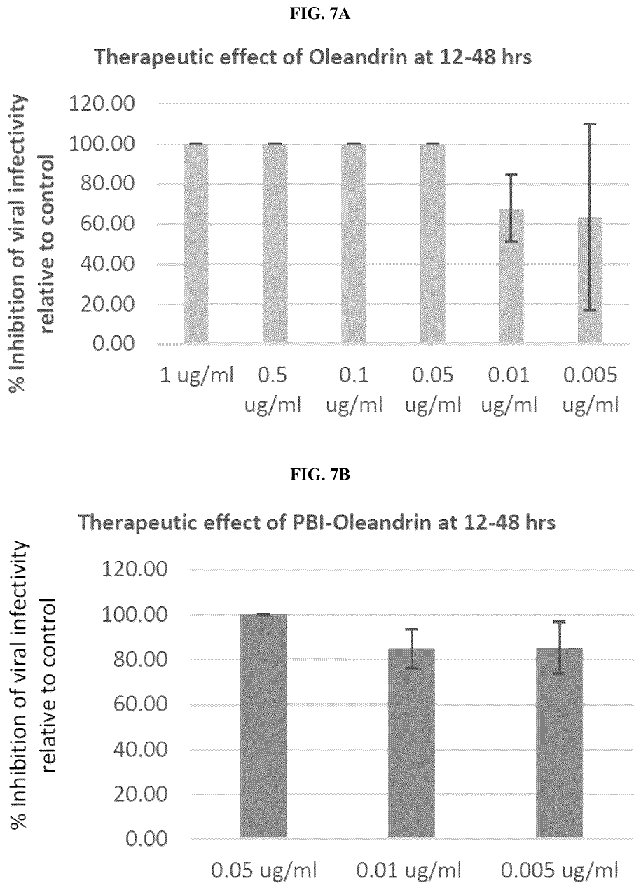

The results in FIG. 7A (oleandrin as sole active) and FIG. 7B (oleander extract containing oleandrin) indicate that a) oleandrin caused a 62-100% reduction in viral infectivity the 24-h time point and the 48-h time point; b) oleandrin is efficacious over the entire concentration range of about 0.005 microg/mL and higher; c) oleandrin should be administered repeatedly, since a single dose is not sufficient to fully stop viral replication; and d) oleandrin is effective at reducing viral infectivity of progeny virions. The results also indicated that oleandrin at concentrations of up to 1.0 microg/mL is not toxic to BT cells.

Accordingly, the invention provides a method of treating BRSV infection in an animal, the method comprising administering a therapeutically effective amount of oleandrin to said animal having said infection.

The prophylactic efficacy of oleandrin and an oleandrin-containing extract against BRSV was evaluated according to Example 25. BT cells were plated 48 hours prior to the assay. At the time the of the assay, the media was removed from each well and replaced with media containing the desired concentrations of Oleandrin or PBI-05204 dissolved in DMSO, or matched concentrations of DMSO-only. Oleandrin, PBI-extract, and DMSO dilutions were made fresh prior to the assay. Plates were incubated with product for 30 minutes, then BRSV virus at an MOI of 0.01 was added to each well. Virus was incubated on the plates for 1 hour and then removed. Plates were washed gently with DPBS and 2 ml of virus maintenance medium containing Oleandrin, PBI-extract, or DMSO-only was added to each well. Samples were removed at each time point (24 and 48 hr) and aliquoted into two cryovials. Virus isolations were performed immediately on samples collected at each time point and the aliquots were then frozen at −80° C. Samples were submitted for RT-qPCR analysis.

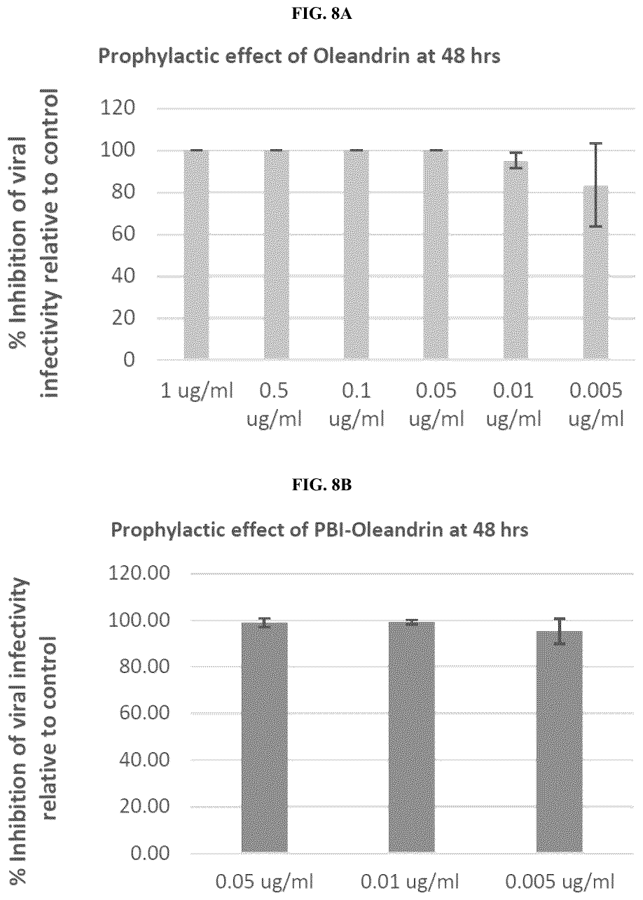

The results in FIG. 8A (oleandrin as sole active) and FIG. 8B (oleander extract containing oleandrin) indicate that a) a 30 min preincubation of cells with oleandrin over the concentration range of 0.005 to 1 microg/ml prior to infection with BRSV resulted in a 82% to 100% inhibition of viral infectivity when measured 48 hr post virus infection; and b) the data in FIG. 8B demonstrate that a 30 min preincubation of cells with PBI-oleandrin prior to infection of cells with BRSV resulted in a 93% to 99% inhibition of viral infectivity when measured 48 hr post virus infection.

Accordingly, the invention provides a method of preventing progression of BRSV infection to a disease state, the method comprising administering a therapeutically effective amount of oleandrin to an animal having said infection.

The concentrations of oleandrin evaluated in the assays are clinically relevant in terms of dosing and plasma concentration.

Proof of the safety of the oleandrin-containing composition was further provided by in vitro cellular assays for determining the release of lactate dehydrogenase after exposure of said cells to solutions containing different concentrations of oleandrin. It was determined that up to concentrations of 1 microg/mL, there was no additional toxicity over control vehicle.

The invention thus provides a method of treating viral infection in an animal, the method comprising chronically administering to an animal, having said infection, therapeutically effective doses of cardiac glycoside (cardiac glycoside-containing composition). Chronic administration can be achieved by repeatedly administering one or more (plural) therapeutically effective doses of cardiac glycoside (cardiac glycoside-containing composition). One or more doses may be administered per day for one or more days per week and optionally for one or more weeks per month and optionally for one or more months per year.

Accordingly, the invention provides a method of treating viral infection in an animal in need thereof comprising administering to the animal one or more doses of antiviral composition comprising a) oleandrin; or b) oleandrin and one or more other compounds extracted from Nerium species. The oleandrin may be present as part of an extract of Nerium species, which extract may be a a) supercritical fluid extract; b) hot-water extract; c) organic solvent extract; d) aqueous organic solvent extract; e) extract using supercritical fluid, optionally plus at least one organic solvent (extraction modifier); f) extract using subcritical liquid, optionally plus at least one organic solvent (extraction modifier); or g) any combination of any two or more of said extracts.

PBI-05204 (as described herein and in U.S. Pat. No. 8,187,644 B2 to Addington, which issued May 29, 2012, U.S. Pat. No. 7,402,325 B2 to Addington, which issued Jul. 22, 2008, U.S. Pat. No. 8,394,434 B2 to Addington et al, which issued Mar. 12, 2013, the entire disclosures of which are hereby incorporated by reference) comprises cardiac glycoside (oleandrin, OL) and triterpenes (oleanolic acid (OA), ursolic acid (UA) and betulinic acid (BA)) as the primary pharmacologically active components. The molar ratio of OL to total triterpene is about 1:(10-96). The molar ratio of OA:UA:BA is about 7.8:7.4:1. The combination of OA, UA and BA in PBI-05204 increases the antiviral activity of oleandrin when compared on an OL equimolar basis. PBI-04711 is a fraction of PBI-05204, but it does not contain cardiac glycoside (OL). The molar ratio of OA:UA:BA in PBI-04711 is about 3:2.2:1. PBI-04711 also possesses antiviral activity. Accordingly, an antiviral composition comprising OL, OA, UA, and BA is more efficacious than a composition comprising OL as the sole active ingredient based upon an equimolar content of OL. In some embodiments, the molar ratios of the individual triterpenes to oleandrin range as follows: about 2-8 (OA):about 2-8 (UA):about 0.1-1 (BA):about 0.5-1.5 (OL); or about 3-6 (OA):about 3-6 (UA):about 0.3-8 (BA):about 0.7-1.2 (OL); or about 4-5 (OA):about 4-5 (UA):about 0.4-0.7 (BA):about 0.9-1.1 (OL); or about 4.6 (OA):about 4.4 (UA):about 0.6 (BA):about 1 (OL).

Antiviral compositions comprising oleandrin as the sole antiviral agent are within the scope of the invention. Antiviral compositions comprising digoxin as the sole antiviral agent are within the scope of the invention.

Antiviral compositions comprising oleandrin and plural triterpenes as the antiviral agents are within the scope of the invention. In some embodiments, the antiviral composition comprises oleandrin, oleanolic acid (free acid, salt, derivative or prodrug thereof), ursolic acid (free acid, salt, derivative or prodrug thereof), and betulinic acid (free acid, salt, derivative or prodrug thereof). The molar ratios of the compounds is as described herein.

Antiviral compositions comprising plural triterpenes as the primary active ingredients (meaning excluding steroid, cardiac glycoside and pharmacologically active components) are also within the scope of the invention. As noted above, PBI-04711 comprises OA, UA and BA as the primary active ingredients, and it exhibits antiviral activity. In some embodiments, a triterpene-based antiviral composition comprises OA, UA and BA, each of which is independently selected upon each occurrence from its free acid form, salt form, deuterated form and derivative form.

PBI-01011 is an improved triterpene-based antiviral composition comprising OA, UA and BA, wherein the molar ratio of OA:UA:BA is about 9-12:up to about 2:up to about 2, or about 10:about 1:about 1, or about 9-12:about 0.1-2:about 0.1-2, or about 9-11:about 0.5-1.5:about 0.5-1.5, or about 9.5-10.5:about 0.75-1.25:about 0.75-1.25, or about 9.5-10.5:about 0.8-1.2:about 0.8-1.2, or about 9.75-10.5:about 0.9-1.1:about 0.9-1.1.

In some embodiments, an antiviral composition comprises at least oleanolic acid (free acid, salt, derivative or prodrug thereof) and ursolic acid (free acid, salt, derivative or prodrug thereof) present at a molar ratio of OA to UA as described herein. OA is present in large molar excess over UA.

In some embodiments, an antiviral composition comprises at least oleanolic acid (free acid, salt, derivative or prodrug thereof) and betulinic acid (free acid, salt, derivative or prodrug thereof) present at a molar ratio of OA to BA as described herein. OA is present in large molar excess over BA.

In some embodiments, an antiviral composition comprises at least oleanolic acid (free acid, salt, derivative or prodrug thereof), ursolic acid (free acid, salt, derivative or prodrug thereof), and betulinic acid (free acid, salt, derivative or prodrug thereof) present at a molar ratio of OA to UA to BA as described herein. OA is present in large molar excess over both UA and BA.

In some embodiments, a triterpene-based antiviral composition excludes cardiac glycoside.

In general, an animal having Arterviridae infection, Flaviviridae infection, Coronaviridae infection, or Paramyxoviridae infection is treated as follows. The animal is evaluated to determine whether said subject is infected with said virus. Administration of antiviral composition is indicated. Initial doses of antiviral composition are administered to the animal according to a prescribed dosing regimen for a period of time (a treatment period). The animal's clinical response and level of therapeutic response are determined periodically. If the level of therapeutic response is too low at one dose, then the dose is escalated according to a predetermine dose escalation schedule until the desired level of therapeutic response in the animal is achieved. Treatment of the animal with antiviral composition is continued as needed. The dose or dosing regimen can be adjusted as needed until the animal reaches the desired clinical endpoint(s) such as cessation of the infection itself, reduction in infection-associated symptoms, and/or a reduction in the progression of the infection.

If a clinician intends to treat an animal having viral infection with a combination of an antiviral composition and one or more other therapeutic agents, and it is known that the viral infection, which the animal has, is at least partially therapeutically responsive to treatment with said one or more other therapeutic agents, then the present method invention comprises: administering to the animal in need thereof a therapeutically relevant dose of antiviral composition and a therapeutically relevant dose of said one or more other therapeutic agents, wherein the antiviral composition is administered according to a first dosing regimen and the one or more other therapeutic agents is administered according to a second dosing regimen. In some embodiments, the first and second dosing regimens are the same. In some embodiments, the first and second dosing regimens are different.

The antiviral composition(s) of the invention can be administered as primary antiviral therapy, adjunct antiviral therapy, or co-antiviral therapy. Methods of the invention include separate administration or coadministration of the antiviral composition with at least one other known antiviral composition, meaning the antiviral composition of the invention can be administered before, during or after administration of a known antiviral composition (compound(s)) or of a composition for treating symptoms associated with the viral infection. For example, medications used to treat inflammation, vomiting, nausea, headache, fever, diarrhea, nausea, hives, conjunctivitis, malaise, muscle pain, joint pain, seizure, or paralysis can be administered with or separately from the antiviral composition of the invention.

The one or more other therapeutic agents can be administered at doses and according to dosing regimens that are clinician-recognized as being therapeutically effective or at doses that are clinician-recognized as being sub-therapeutically effective. The clinical benefit and/or therapeutic effect provided by administration of a combination of antiviral composition and one or more other therapeutic can be additive or synergistic, such level of benefit or effect being determined by comparison of administration of the combination to administration of the individual antiviral composition component(s) and one or more other therapeutic agents. The one or more other therapeutic agents can be administered at doses and according to dosing regimens as suggested or described by the Food and Drug Administration (Center for Veterinary Medicine), World Health Organization, European Medicines Agency (Veterinary Medicines Division), Australian Pesticides and Veterinary Medicines Authority (APVMA), Pan American Health Organization (Veterinary Public Health Program), Agricultural Compounds and Veterinary Medicines Authority (New Zealand) or the various Ministries of Health worldwide.

The antiviral compound(s) (triterpene(s), cardiac glycoside(s), etc.) present in the pharmaceutical composition can be present in their unmodified form, salt form, derivative form or a combination thereof. As used herein, the term “derivative” is taken to mean: a) a chemical substance that is related structurally to a first chemical substance and theoretically derivable from it; b) a compound that is formed from a similar first compound or a compound that can be imagined to arise from another first compound, if one atom of the first compound is replaced with another atom or group of atoms; c) a compound derived or obtained from a parent compound and containing essential elements of the parent compound; or d) a chemical compound that may be produced from first compound of similar structure in one or more steps. For example, a derivative may include a deuterated form, oxidized form, dehydrated, unsaturated, polymer conjugated or glycosylated form thereof or may include an ester, amide, lactone, homolog, ether, thioether, cyano, amino, alkylamino, sulfhydryl, heterocyclic, heterocyclic ring-fused, polymerized, pegylated, benzylidenyl, triazolyl, piperazinyl or deuterated form thereof.

As used herein, the term “oleandrin” is taken to mean all known forms of oleandrin unless otherwise specified. Oleandrin can be present in racemic, optically pure or optically enriched form. Nerium sp. plant material can be obtained, for example, from commercial plant suppliers such as Aldridge Nursery, Atascosa, Texas.

The supercritical fluid (SCF) extract can be prepared as detailed in U.S. Pat. Nos. 7,402,325, 8,394,434, 8,187,644, or PCT International Publication No. WP 2007/016176 A2, the entire disclosures of which are hereby incorporated by reference. Extraction can be conducted with supercritical carbon dioxide in the presence or absence of a modifier (organic solvent) such as ethanol.

A hot-water extract is available under the tradename ANVIRZEL™ (Nerium Biotechnology, Inc., San Antonio, TX; Salud Integral Medical Clinic, Tegucigalpa, Honduras; www.saludintegral.com; www.anvirzel.com) as a liquid dosage form. ANVIRZEL™ comprises oleandrin, oleandrigenin, polysaccharides extracted (hot water extraction) from Nerium oleander. Commercially available vials comprise about 150 mg of oleander extract as a freeze-dried powder (prior to reconstitution with water before administration) which comprises about 200 to about 900 microg of oleandrin, about 500 to about 700 microg of oleandrigenin, and polysaccharides extracted from Nerium oleander. Said vials may also include pharmaceutical excipients such as at least one osmotic agent, e.g. mannitol, sodium chloride, at least one buffering agent, e.g. sodium ascorbate with ascorbic acid, at least one preservative, e.g. propylparaben, methylparaben.

Other extracts containing cardiac glycoside, especially oleandrin, can be prepared by various different processes. An extract can be prepared according to the process developed by Dr. Huseyin Ziya Ozel (U.S. Pat. No. 5,135,745) describes a procedure for the preparation of a hot water extract. The aqueous extract reportedly contains several polysaccharides with molecular weights varying from 2 KD to 30 KD, oleandrin, oleandrigenin, odoroside and neritaloside. The polysaccharides reportedly include acidic homopolygalacturonans or arabinogalaturonans. U.S. Pat. No. 5,869,060 to Selvaraj et al. discloses hot water extracts of Nerium species and methods of production thereof, e.g. Example 2. The resultant extract can then be lyophilized to produce a powder. U.S. Pat. No. 6,565,897 (U.S. Pregrant Publication No. 20020114852 and PCT International Publication No. WO 2000/016793 to Selvaraj et al.) discloses a hot-water extraction process for the preparation of a substantially sterile extract. Erdemoglu et al. (J. Ethnopharmacol. (2003) November 89(1), 123-129) discloses results for the comparison of aqueous and ethanolic extracts of plants, including Nerium oleander, based upon their anti-nociceptive and anti-inflammatory activities. Organic solvent extracts of Nerium oleander are disclosed by Adome et al. (Afr. Health Sci. (2003) August 3(2), 77-86; ethanolic extract), el-Shazly et al. U. Egypt Soc. Parasitol. (1996), August 26(2), 461-473; ethanolic extract), Begum et al. (Phytochemistry (1999) February 50(3), 435-438; methanolic extract), Zia et al. (J. Ethnolpharmacol. (1995) November 49(1), 33-39; methanolic extract), and Vlasenko et al. (Farmatsiia. (1972) September-October 21(5), 46-47; alcoholic extract). U.S. Pregrant Patent Application Publication No. 20040247660 to Singh et al. discloses the preparation of a protein stabilized liposomal formulation of oleandrin for use in the treatment of cancer. U.S. Pregrant Patent Application Publication No. 20050026849 to Singh et al. discloses a water soluble formulation of oleandrin containing a cyclodextrin. U.S. Pregrant Patent Application Publication No. 20040082521 to Singh et al. discloses the preparation of protein stabilized nanoparticle formulations of oleandrin from the hot-water extract.

Oleandrin may also be obtained from extracts of suspension cultures derived from Agrobacterium tumefaciens-transformed calli (Ibrahim et al., “Stimulation of oleandrin production by combined Agrobacterium tumefaciens mediated transformation and fungal elicitation in Nerium oleander cell cultures” in Enz. Microbial Techno. (2007), 41(3), 331-336, the entire disclosure of which is hereby incorporated by reference). Hot water, organic solvent, aqueous organic solvent, or supercritical fluid extracts of agrobacterium may be used according to the invention.

Oleandrin may also be obtained from extracts of Nerium oleander microculture in vitro, whereby shoot cultures can be initiated from seedlings and/or from shoot apices of the Nerium oleander cultivars Splendens Giganteum, Revanche or Alsace, or other cultivars (Vila et al., “Micropropagation of Oleander (Nerium oleander L.)” in HortScience (2010), 45(1), 98-102, the entire disclosure of which is hereby incorporated by reference). Hot water, organic solvent, aqueous organic solvent, or supercritical fluid extracts of microcultured Nerium sp. may be used according to the invention.

The extracts also differ in their polysaccharide and carbohydrate content. The hot water extract contains 407.3 glucose equivalent units of carbohydrate relative to a standard curve prepared with glucose while analysis of the SCF CO₂ extract found carbohydrate levels that were found in very low levels that were below the limit of quantitation. The amount of carbohydrate in the hot water extract of Nerium oleander was, however, at least 100-fold greater than that in the SCF CO₂ extract. The polysaccharide content of the SCF extract can be 0%, <0.5%, <0.1%, <0.05%, or <0.01% wt. In some embodiments, the SCF extract excludes polysaccharide obtained during extraction of the plant mass.

| | Polysaccharide content |
|---|---|
| Nerium oleander | (μg glucose equivalents/mg |
| preparation | of plant extract) |
| Hot water extract | 407.3 ± 6.3 |
| SCF CO₂ extract | BLQ (below limit of quantitation) |

The partial compositions of the SCF CO₂ extract and hot water extract were determined by DART TOF-MS (Direct Analysis in Real Time Time of Flight Mass Spectrometry) on a JEOL AccuTOF-DART mass spectrometer (JEOL USA, Peabody, MA, USA).

The SCF extract of Nerium species or Thevetia species is a mixture of pharmacologically active compounds, such as oleandrin and triterpenes. The extract obtained by the SCF process is a substantially water-insoluble, viscous semi-solid (after solvent is removed) at ambient temperature. The SCF extract comprises many different components possessing a variety of different ranges of water solubility. The extract from a supercritical fluid process contains by weight a theoretical range of 0.9% to 2.5% wt of oleandrin or 1.7% to 2.1% wt of oleandrin or 1.7% to 2.0% wt of oleandrin. SCF extracts comprising varying amount of oleandrin have been obtained. In one embodiment, the SCF extract comprises about 2% by wt. of oleandrin. The SCF extract contains a 3-10 fold higher concentration of oleandrin than the hot-water extract. This was confirmed by both HPLC as well as LC/MS/MS (tandem mass spectrometry) analyses.

The SCF extract comprises oleandrin and the triterpenes oleanolic acid, betulinic acid and ursolic acid and optionally other components as described herein. The content of oleandrin and the triterpenes can vary from batch to batch; however, the degree of variation is not excessive. For example, a batch of SCF extract (PBI-05204) was analyzed for these four components and found to contain the following approximate amounts of each.

| | | Oleanolic | Ursolic | Betulinic |
|---|---|---|---|---|
| | Oleandrin | acid | acid | acid |
| Content of component | 20 | 73 | 69 | 9.4 |
| (mg/g of SCF extract) | | | | |
| Content of component | 2 | 7.3 | 6.9 | 0.94 |
| (% wt WRT g of SCT | | | | |
| extract) | | | | |
| Content of component | 34.7 | 160 | 152 | 20.6 |
| (mmole/g of SCF | | | | |
| extract) | | | | |
| Molar ratio of | 1 | 4.6 | 4.4 | 0.6 |
| component WRT | | | | |
| oleandrin | | | | |

WRT denotes “with respect to”.

The content of the individual components may vary by ±25%, ±20%, ±15%, ±10% or ±5% relative to the values indicated. Accordingly, the content of oleandrin in the SCF extract would be in the range of 20 mg±5 mg (which is ±25% of 20 mg) per mg of SCF extract.

Oleandrin, oleanolic acid, ursolic acid, betulinic acid and derivatives thereof can also be purchased from Sigma-Aldrich (www.sigmaaldrich.com; St. Louis, MO, USA). Digoxin is commercially available from HIKMA Pharmaceuticals International LTD (NDA N012648, elixir, 0.05 mg/mL; tablet, 0.125 mg, 0.25 mg), VistaPharm Inc. (NDA A213000, elixir, 0.05 mg/mL), Sandoz Inc. (NDA A040481, injectable, 0.25 mg/mL), West-Ward Pharmaceuticals International LTD (NDA A083391, injectable, 0.25 mg/mL), Covis Pharma BV (NDA N009330, 0.1 mg/mL, 0.25 mg/mL), Impax Laboratories (NDA A078556, tablet, 0.125 mg, 0.25 mg), Jerome Stevens Pharmaceuticals Inc. (NDA A076268, tablet, 0.125 mg, 0.25 mg), Mylan Pharmaceuticals Inc. (NDA A040282, tablet, 0.125 mg, 0.25 mg), Sun Pharmaceutical Industries Inc. (NDA A076363, tablet, 0.125 mg, 0.25 mg), Concordia Pharmaceuticals Inc. (NDA A020405, tablet, 0.0625, 0.125 mg, 0.1875 mg, 0.25 mg, 0.375 mg, 0.5 mg, LANOXIN), GlaxoSmithKline LLC (NDA 018118, capsule, 0.05 mg, 0.1 mg, 0.15 mg, 0.2 mg, LANOXICAPS).

As used herein, the individually named triterpenes can independently be selected upon each occurrence in their native (unmodified, free acid) form, in their salt form, in derivative form, prodrug form, or a combination thereof. Compositions containing and methods employing deuterated forms of the triterpenes are also within the scope of the invention.

Oleanolic acid derivatives, prodrugs and salts are disclosed in US 20150011627 A1 to Gribble et al. which published Jan. 8, 2015, US 20140343108 A1 to Rong et al which published Nov. 20, 2014, US 20140343064 A1 to Xu et al. which published Nov. 20, 2014, US 20140179928 A1 to Anderson et al. which published Jun. 26, 2014, US 20140100227 A1 to Bender et al. which published Apr. 10, 2014, US 20140088188 A1 to Jiang et al. which published Mar. 27, 2014, US 20140088163 A 1 to Jiang et al. which published Mar. 27, 2014, US 20140066408 A1 to Jiang et al. which published Mar. 6, 2014, US 20130317007 A1 to Anderson et al. which published Nov. 28, 2013, US 20130303607 A1 to Gribble et al. which published Nov. 14, 2013, US 20120245374 to Anderson et al. which published Sep. 27, 2012, US 20120238767 A1 to Jiang et al. which published Sep. 20, 2012, US 20120237629 A1 to Shode et al. which published Sep. 20, 2012, US 20120214814 A1 to Anderson et al. which published Aug. 23, 2012, US 20120165279 A1 to Lee et al. which published Jun. 28, 2012, US 20110294752 A1 to Arntzen et al. which published Dec. 1, 2011, US 20110091398 A1 to Majeed et al. which published Apr. 21, 2011, US 20100189824 A1 to Arntzen et al. which published Jul. 29, 2010, US 20100048911 A1 to Jiang et al. which published Feb. 25, 2010, and US 20060073222 A1 to Arntzen et al. which published Apr. 6, 2006, the entire disclosures of which are hereby incorporated by reference.

Ursolic acid derivatives, prodrugs and salts are disclosed in US 20150011627 A1 to Gribble et al. which published Jan. 8, 2015, US 20130303607 A1 to Gribble et al. which published Nov. 14, 2013, US 20150218206 A1 to Yoon et al. which published Aug. 6, 2015, U.S. Pat. No. 6,824,811 to Fritsche et al. which issued Nov. 30, 2004, U.S. Pat. No. 7,718,635 to Ochiai et al. which issued May 8, 2010, U.S. Pat. No. 8,729,055 to Lin et al. which issued May 20, 2014, and U.S. Pat. No. 9,120,839 to Yoon et al. which issued Sep. 1, 2015, the entire disclosures of which are hereby incorporated by reference.

Betulinic acid derivatives, prodrugs and salts are disclosed in US 20150011627 A1 to Gribble et al. which published Jan. 8, 2015, US 20130303607 A1 to Gribble et al. which published Nov. 14, 2013, US 20120237629 A1 to Shode et al. which published Sep. 20, 2012, US 20170204133 A1 to Regueiro-Ren et al. which published Jul. 20, 2017, US 20170096446 A1 to Nitz et al. which published Apr. 6, 2017, US 20150337004 A1 to Parthasaradhi Reddy et al. which published Nov. 26, 2015, US 20150119373 A1 to Parthasaradhi Reddy et al. which published Apr. 30, 2015, US 20140296546 A1 to Yan et al. which published Oct. 2, 2014, US 20140243298 A1 to Swidorski et al. which published Aug. 28, 2014, US 20140221328 A1 to Parthasaradhi Reddy et al. which published Aug. 7, 2014, US 20140066416 A1 tp Leunis et al. which published Mar. 6, 2014, US 20130065868 A1 to Durst et al. which published Mar. 14, 2013, US 20130029954 A1 to Regueiro-Ren et al. which published Jan. 31, 2013, US 20120302530 A1 to Zhang et al. which published Nov. 29, 2012, US 20120214775 A1 to Power et al. which published Aug. 23, 2012, US 20120101149 A1 to Honda et al. which published Apr. 26, 2012, US 20110224182 to Bullock et al. which published Sep. 15, 2011, US 20110313191 A1 to Hemp et al. which published Dec. 22, 2011, US 20110224159 A1 to Pichette et al. which published Sep. 15, 2011, US 20110218204 to Parthasaradhi Reddy et al. which published Sep. 8, 2011, US 20090203661 A1 to Safe et al. which published Aug. 13, 2009, US 20090131714 A1 to Krasutsky et al. which published May 21, 2009, US 20090076290 to Krasutsky et al. which published Mar. 19, 2009, US 20090068257 A1 to Leunis et al. which published Mar. 12, 2009, US 20080293682 to Mukherjee et al. which published Nov. 27, 2008, US 20070072835 A1 to Pezzuto et al. which published Mar. 29, 2007, US 20060252733 A1 to Jansen et al. which published Nov. 9, 2006, and US 2006025274 A1 to O'Neill et al. which published Nov. 9, 2006, the entire disclosures of which are hereby incorporated by reference.

Since viral infection may affect multiple organs simultaneously and cause multiple organ failure, it may be advantageous to administer the composition by more than one route.

The antiviral composition can be formulated in any suitable pharmaceutically acceptable dosage form. Parenteral, otic, ophthalmic, nasal, inhalable, buccal, sublingual, enteral, topical, oral, peroral, and injectable dosage forms are particularly useful. Particular dosage forms include a solid or liquid dosage forms. Exemplary suitable dosage forms include tablet, capsule, pill, caplet, troche, sache, solution, suspension, dispersion, vial, bag, bottle, injectable liquid, i.v. (intravenous), i.m. (intramuscular) or i.p. (intraperitoneal) administrable liquid and other such dosage forms known to the artisan of ordinary skill in the pharmaceutical sciences.

Suitable dosage forms for administering oleandrin (or digoxin) to an animal can be made according to known procedures wherein oleandrin (or digoxin) is used in place of another drug: Klink et al. (“Formulations of Veterinary Dosage Forms” in Development and Formulation of Veterinary Dosage Forms, 2^{nd} ed., Eds. G. E. Hardee and J. D. Baggot, New York, CRC Press, 1998), Foster et al. (“Veterinary Dosage Forms” in Encyclopedia of Pharmaceutical Science and Technology, 4^{th} ed., Eds. J. Swarbrick, New York, CRC Press, 2015).

An effective amount or therapeutically relevant amount of antiviral compound (cardiac glycoside, triterpene or combinations thereof) is specifically contemplated. By the term “effective amount”, it is understood that a pharmaceutically effective amount is contemplated. A pharmaceutically effective amount is the amount or quantity of active ingredient which is enough for the required or desired therapeutic response, or in other words, the amount, which is sufficient to elicit an appreciable biological response when, administered to an animal. The appreciable biological response may occur as a result of administration of single or multiple doses of an active substance. A dose may comprise one or more dosage forms. It will be understood that the specific dose level for any animal will depend upon a variety of factors including the indication being treated, severity of the indication, animal health, age, gender, weight, diet, pharmacological response, the specific dosage form employed, and other such factors.

The desired dose for oral administration is up to 5 dosage forms although as few as one and as many as ten dosage forms may be administered as a single dose. Doses will be administered according to dosing regimens that may be predetermined and/or tailored to achieve specific therapeutic response or clinical benefit in an animal.

The cardiac glycoside can be present in a dosage form in an amount sufficient to provide an animal with an initial dose of oleandrin of about 20 to about 100 microg, about 12 microg to about 300 microg, or about 12 microg to about 120 microg. For example, a dosage form can comprise about 20 of oleandrin to about 100 microg, about 0.01 microg to about 100 mg or about 0.01 microg to about 100 microg oleandrin, oleandrin extract or extract of Nerium sp. containing oleandrin.

The antiviral can be included in an oral dosage form. Some embodiments of the dosage form are not enteric coated and release their charge of antiviral composition within a period of 0.5 to 1 hours or less. Some embodiments of the dosage form are enteric coated and release their charge of antiviral composition downstream of the stomach, such as from the jejunum, ileum, small intestine, and/or large intestine (colon). Enterically coated dosage forms will release antiviral composition into the systemic circulation within 1-10 hr after oral administration.

The antiviral composition can be included in a rapid release, immediate release, controlled release, sustained release, prolonged release, extended release, burst release, continuous release, slow release, or pulsed release dosage form or in a dosage form that exhibits two or more of those types of release. The release profile of antiviral composition from the dosage form can be a zero order, pseudo-zero, first order, pseudo-first order or sigmoidal release profile. The plasma concentration profile for triterpene in an animal to which the antiviral composition is administered can exhibit one or more maxima.

The anticipated oleandrin plasma concentration (Cmax or Cavg as measure in a 24-h period) will be in the range of about 0.005 to about 5 ng/ml, about 0.005 to about 4 ng/mL, about 0.005 to about 3 ng/mL, about 0.005 to about 2 ng/mL, or about 0.005 to about 2 ng/mL. A veterinary clinician will be used known dose escalation and de-escalation protocols to determine the appropriate dose of oleandrin or digoxin to be safely administered per day.

It should be noted that a compound herein might possess one or more functions in a composition or formulation of the invention. For example, a compound might serve as both a surfactant and a water miscible solvent or as both a surfactant and a water immiscible solvent.

A liquid composition can comprise one or more pharmaceutically acceptable liquid carriers. The liquid carrier can be an aqueous, non-aqueous, polar, non-polar, and/or organic carrier. Liquid carriers include, by way of example and without limitation, a water miscible solvent, water immiscible solvent, water, buffer and mixtures thereof.

As used herein, the terms “water soluble solvent” or “water miscible solvent”, which terms are used interchangeably, refer to an organic liquid which does not form a biphasic mixture with water or is sufficiently soluble in water to provide an aqueous solvent mixture containing at least five percent of solvent without separation of liquid phases. The solvent is suitable for administration to animals. Exemplary water soluble solvents include, by way of example and without limitation, PEG (poly(ethylene glycol)), PEG 400 (poly(ethylene glycol having an approximate molecular weight of about 400), ethanol, acetone, alkanol, alcohol, ether, propylene glycol, glycerin, triacetin, poly(propylene glycol), PVP (poly(vinyl pyrrolidone)), dimethylsulfoxide, N,N-dimethylformamide, formamide, N,N-dimethylacetamide, pyridine, propanol, N-methylacetamide, butanol, soluphor (2-pyrrolidone), pharmasolve (N-methyl-2-pyrrolidone).

As used herein, the terms “water insoluble solvent” or “water immiscible solvent”, which terms are used interchangeably, refer to an organic liquid which forms a biphasic mixture with water or provides a phase separation when the concentration of solvent in water exceeds five percent. The solvent is suitable for administration to animals. Exemplary water insoluble solvents include, by way of example and without limitation, medium/long chain triglycerides, oil, castor oil, corn oil, vitamin E, vitamin E derivative, oleic acid, fatty acid, olive oil, softisan 645 (Diglyceryl Caprylate/Caprate/Stearate/Hydroxy stearate adipate), miglyol, captex (Captex 350: Glyceryl Tricaprylate/Caprate/Laurate triglyceride; Captex 355: Glyceryl Tricaprylate/Caprate triglyceride; Captex 355 EP/NF: Glyceryl Tricaprylate/Caprate medium chain triglyceride).

Suitable solvents are listed in the “International Conference on Harmonisation of Technical Requirements for Registration of Pharmaceuticals for Human Use (ICH) guidance for industry Q3C Impurities: Residual Solvents” (1997), which makes recommendations as to what amounts of residual solvents are considered safe in pharmaceuticals. Exemplary solvents are listed as class 2 or class 3 solvents. Class 3 solvents include, for example, acetic acid, acetone, anisole, 1-butanol, 2-butanol, butyl acetate, tert-butlymethyl ether, cumene, ethanol, ethyl ether, ethyl acetate, ethyl formate, formic acid, heptane, isobutyl acetate, isopropyl acetate, methyl acetate, methyl-1-butanol, methylethyl ketone, methylisobutyl ketone, 2-methyl-1-propanol, pentane, 1-pentanol, 1-propanol, 2-propanol, or propyl acetate.

Other materials that can be used as water immiscible solvents in the invention include: Captex 100: Propylene Glycol Dicaprate; Captex 200: Propylene Glycol Dicaprylate/Dicaprate; Captex 200 P: Propylene Glycol Dicaprylate/Dicaprate; Propylene Glycol Dicaprylocaprate; Captex 300: Glyceryl Tricaprylate/Caprate; Captex 300 EP/NF: Glyceryl Tricaprylate/Caprate Medium Chain Triglycerides; Captex 350: Glyceryl Tricaprylate/Caprate/Laurate; Captex 355: Glyceryl Tricaprylate/Caprate; Captex 355 EP/NF: Glyceryl Tricaprylate/Caprate Medium Chain Triglycerides; Captex 500: Triacetin; Captex 500 P: Triacetin (Pharmaceutical Grade); Captex 800: Propylene Glycol Di (2-Ethythexanoate); Captex 810 D: Glyceryl Tricaprylate/Caprate/Linoleate; Captex 1000: Glyceryl Tricaprate; Captex CA: Medium Chain Triglycerides; Captex MCT-170: Medium Chain Triglycerides; Capmul GMO: Glyceryl Monooleate; Capmul GMO-50 EP/NF: Glyceryl Monooleate; Capmul MCM: Medium Chain Mono- & Diglycerides; Capmul MCM C8: Glyceryl Monocaprylate; Capmul MCM C10: Glyceryl Monocaprate; Capmul PG-8: Propylene Glycol Monocaprylate; Capmul PG-12: Propylene Glycol Monolaurate; Caprol 10G100: Decaglycerol Decaoleate; Caprol 3GO: Triglycerol Monooleate; Caprol ET: Polyglycerol Ester of Mixed Fatty Acids; Caprol MPGO: Hexaglycerol Dioleate; Caprol PGE 860: Decaglycerol Mono-, Dioleate.

As used herein, a “surfactant” refers to a compound that comprises polar or charged hydrophilic moieties as well as non-polar hydrophobic (lipophilic) moieties; i.e., a surfactant is amphiphilic. The term surfactant may refer to one or a mixture of compounds. A surfactant can be a solubilizing agent, an emulsifying agent or a dispersing agent. A surfactant can be hydrophilic or hydrophobic.

The hydrophilic surfactant can be any hydrophilic surfactant suitable for use in pharmaceutical compositions. Such surfactants can be anionic, cationic, zwitterionic or non-ionic, although non-ionic hydrophilic surfactants are presently preferred. As discussed above, these non-ionic hydrophilic surfactants will generally have HLB values greater than about 10. Mixtures of hydrophilic surfactants are also within the scope of the invention.

Similarly, the hydrophobic surfactant can be any hydrophobic surfactant suitable for use in pharmaceutical compositions. In general, suitable hydrophobic surfactants will have an HLB value less than about 10. Mixtures of hydrophobic surfactants are also within the scope of the invention.

Examples of additional suitable solubilizer include: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol, available commercially from BASF under the trade name Tetraglycol) or methoxy PEG (Union Carbide); amides, such as 2-pyrrolidone, 2-piperidone, caprolactam, N-alkylpyrrolidone, N-hydroxyalkylpyrrolidone, N-alkylpiperidone, N-alkylcaprolactam, dimethylacetamide, and polyvinypyrrolidone; esters, such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, caprolactone and isomers thereof, valerolactone and isomers thereof, butyrolactone and isomers thereof; and other solubilizers known in the art, such as dimethyl acetamide, dimethyl isosorbide (Arlasolve DMI (ICI)), N-methyl pyrrolidones (Pharmasolve (ISP)), monooctanoin, diethylene glycol nonoethyl ether (available from Gattefosse under the trade name Transcutol), and water. Mixtures of solubilizers are also within the scope of the invention.

Except as indicated, compounds mentioned herein are readily available from standard commercial sources.

Although not necessary, the composition or formulation may further comprise one or more chelating agents, one or more preservatives, one or more antioxidants, one or more adsorbents, one or more acidifying agents, one or more alkalizing agents, one or more antifoaming agents, one or more buffering agents, one or more colorants, one or more electrolytes, one or more salts, one or more stabilizers, one or more tonicity modifiers, one or more diluents, or a combination thereof.

The composition of the invention can also include oils such as fixed oils, peanut oil, sesame oil, cottonseed oil, corn oil and olive oil; fatty acids such as oleic acid, stearic acid and isostearic acid; and fatty acid esters such as ethyl oleate, isopropyl myristate, fatty acid glycerides and acetylated fatty acid glycerides. The composition can also include alcohol such as ethanol, isopropanol, hexadecyl alcohol, glycerol and propylene glycol; glycerol ketals such as 2,2-dimethyl-1,3-dioxolane-4-methanol; ethers such as poly(ethylene glycol) 450; petroleum hydrocarbons such as mineral oil and petrolatum; water; a pharmaceutically suitable surfactant, suspending agent or emulsifying agent; or mixtures thereof.

It should be understood that the compounds used in the art of pharmaceutical formulation generally serve a variety of functions or purposes. Thus, if a compound named herein is mentioned only once or is used to define more than one term herein, its purpose or function should not be construed as being limited solely to that named purpose(s) or function(s).

One or more of the components of the formulation can be present in its free base, free acid or pharmaceutically or analytically acceptable salt form. As used herein, “pharmaceutically or analytically acceptable salt” refers to a compound that has been modified by reacting it with an acid as needed to form an ionically bound pair. Examples of acceptable salts include conventional non-toxic salts formed, for example, from non-toxic inorganic or organic acids. Suitable non-toxic salts include those derived from inorganic acids such as hydrochloric, hydrobromic, sulfuric, sulfonic, sulfamic, phosphoric, nitric and others known to those of ordinary skill in the art. The salts prepared from organic acids such as amino acids, acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and others known to those of ordinary skill in the art. On the other hand, where the pharmacologically active ingredient possesses an acid functional group, a pharmaceutically acceptable base is added to form the pharmaceutically acceptable salt. Lists of other suitable salts are found in Remington's Pharmaceutical Sciences, 17^{th}. ed., Mack Publishing Company, Easton, P A, 1985, p. 1418, the relevant disclosure of which is hereby incorporated by reference.

The phrase “pharmaceutically acceptable” is employed herein to refer to those compounds, materials, compositions, and/or dosage forms which are, within the scope of sound medical judgment, suitable for use in contact with tissues of animals and without excessive toxicity, irritation, allergic response, or any other problem or complication, commensurate with a reasonable benefit/risk ratio.

A dosage form can be made by any conventional means known in the pharmaceutical industry. A liquid dosage form can be prepared by providing at least one liquid carrier and antiviral composition in a container. One or more other excipients can be included in the liquid dosage form. A solid dosage form can be prepared by providing at least one solid carrier and antiviral composition. One or more other excipients can be included in the solid dosage form.

A dosage form can be packaged using conventional packaging equipment and materials. It can be included in a pack, bottle, via, bag, syringe, envelope, packet, blister pack, box, ampoule, or other such container.

The composition of the invention can be included in any dosage form. Particular dosage forms include a solid or liquid dosage forms. Exemplary suitable dosage forms include tablet, capsule, pill, caplet, troche, sache, and other such dosage forms known to the artisan of ordinary skill in the pharmaceutical sciences.

The antiviral composition can further comprise at least one cardiac glycoside-metabolism inhibitor, at least one cardiac glycoside-digestion inhibitor, at least one enzyme inhibitor, or a combination thereof. A cardiac glycoside-metabolism inhibitor is a compound that inhibits metabolism of a cardiac glycoside. A cardiac glycoside-digestion inhibitor is a compound that inhibits digestion of a cardiac glycoside. An enzyme inhibitor is a compound that inhibits an enzyme. The metabolism or digestion can be caused by the animal or one or more microbes in the animal. These categories of inhibitors are herein referred to together more broadly as inhibitors. The purpose of said inhibitors is to reduce the rate of metabolism or digestion of the cardiac glycoside, thereby increasing the plasma concentration half-life of the cardiac glycoside in the animal.

In view of the above description and the examples below, one of ordinary skill in the art will be able to practice the invention as claimed without undue experimentation. The foregoing will be better understood with reference to the following examples that detail certain procedures for the preparation of embodiments of the present invention. All references made to these examples are for the purposes of illustration. The following examples should not be considered exhaustive, but merely illustrative of only a few of the many embodiments contemplated by the present invention.

### Example 1

### Supercritical Fluid Extraction of Powdered Oleander Leaves

### Method A. With Carbon Dioxide.

Powdered oleander leaves were prepared by harvesting, washing, and drying oleander leaf material, then passing the oleander leaf material through a comminuting and dehydrating apparatus such as those described in U.S. Pat. Nos. 5,236,132, 5,598,979, 6,517,015, and 6,715,705. The weight of the starting material used was 3.94 kg.

The starting material was combined with pure CO₂ at a pressure of 300 bar (30 MPa, 4351 psi) and a temperature of 50° C. (122° F.) in an extractor device. A total of 197 kg of CO₂ was used, to give a solvent to raw material ratio of 50:1. The mixture of CO₂ and raw material was then passed through a separator device, which changed the pressure and temperature of the mixture and separated the extract from the carbon dioxide.

The extract (65 g) was obtained as a brownish, sticky, viscous material having a nice fragrance. The color was likely caused by chlorophyll and other residual chromophoric compounds. For an exact yield determination, the tubes and separator were rinsed out with acetone and the acetone was evaporated to give an addition 9 g of extract. The total extract amount was 74 g. Based on the weight of the starting material, the yield of the extract was 1.88%. The content of oleandrin in the extract was calculated using high pressure liquid chromatography and mass spectrometry to be 560.1 mg, or a yield of 0.76%.

### Method B. With Mixture of Carbon Dioxide and Ethanol

Powdered oleander leaves were prepared by harvesting, washing, and drying oleander leaf material, then passing the oleander leaf material through a comminuting and dehydrating apparatus such as those described in U.S. Pat. Nos. 5,236,132, 5,598,979, 6,517,015, and 6,715,705. The weight of the starting material used was 3.85 kg.

The starting material was combined with pure CO₂ and 5% ethanol as a modifier at a pressure of 280 bar (28 MPa, 4061 psi) and a temperature of 50° C. (122° F.) in an extractor device. A total of 160 kg of CO₂ and 8 kg ethanol was used, to give a solvent to raw material ratio of 43.6 to 1. The mixture of CO₂, ethanol, and raw material was then passed through a separator device, which changed the pressure and temperature of the mixture and separated the extract from the carbon dioxide.

The extract (207 g) was obtained after the removal of ethanol as a dark green, sticky, viscous mass obviously containing some chlorophyll. Based on the weight of the starting material, the yield of the extract was 5.38%. The content of oleandrin in the extract was calculated using high pressure liquid chromatography and mass spectrometry to be 1.89 g, or a yield of 0.91%.

### Example 2

### Hot-Water Extraction of Powdered Oleander Leaves.

Hot water extraction is typically used to extract oleandrin and other active components from oleander leaves. Examples of hot water extraction processes can be found in U.S. Pat. Nos. 5,135,745 and 5,869,060.

A hot water extraction was carried out using 5 g of powdered oleander leaves. Ten volumes of boiling water (by weight of the oleander starting material) were added to the powdered oleander leaves and the mixture was stirred constantly for 6 hours. The mixture was then filtered and the leaf residue was collected and extracted again under the same conditions. The filtrates were combined and lyophilized. The appearance of the extract was brown. The dried extract material weighed about 1.44 g. 34.21 mg of the extract material was dissolved in water and subjected to oleandrin content analysis using high pressure liquid chromatography and mass spectrometry. The amount of oleandrin was determined to be 3.68 mg. The oleandrin yield, based on the amount of extract, was calculated to be 0.26%.

### Example 3

### Preparation of Veterinary Compositions.

### Method A. Cremophor-Based Drug Delivery System

The following ingredients were provided in the amounts indicated.

| | | Percent of |
|---|---|---|
| Reagent | | Formulation |
| Name | Function | (% w/w) |
| Antiviral composition | Active agent | 3.7 |
| Vitamin E | Antioxidant | 0.1 |
| Labrasol | Surfactant | 9.2 |
| Ethanol | Co-solvent | 9.6 |
| Cremophor EL | Surfactant | 62.6 |
| Cremophor RH40 | Surfactant | 14.7 |

The excipients were dispensed into a jar and shook in a New Brunswick Scientific C24KC Refrigerated Incubator shaker for 24 hours at 60° C. to ensure homogeneity. The samples were then pulled and visually inspected for solubilization. Both the excipients and antiviral composition were totally dissolved for all formulations after 24 hours.

### Method B. GMO/Cremophor-Based Drug Delivery System

The following ingredients were provided in the amounts indicated.

| | | Percent of |
|---|---|---|
| Reagent | | Formulation |
| Name | Function | (% w/w) |
| antiviral composition | Active agent | 4.7 |
| Vitamin E | Antioxidant | 0.1 |
| Labrasol | Surfactant | 8.5 |
| Ethanol | Co-solvent | 7.6 |
| Cremophor EL | Surfactant | 56.1 |
| Glycerol Monooleate | Surfactant | 23.2 |

The procedure of Method A was followed.

### Method C. Labrasol-Based Drug Delivery System

The following ingredients were provided in the amounts indicated.

| | | Percent of |
|---|---|---|
| Reagent | | Formulation |
| Name | Function | (% w/w) |
| antiviral composition | Active agent | 3.7 |
| Vitamin E | Antioxidant | 0.1 |
| Labrasol | Surfactant | 86.6 |
| Ethanol | Co-solvent | 9.6 |

The procedure of Method A was followed.

### Method D. Vitamin E-TPGS Based Micelle Forming System

The following ingredients were provided in the amounts indicated.

| | | Weight % |
|---|---|---|
| Component | Function | (w/w) |
| Vitamin E | Antioxidant | 1.0 |
| Vitamin E TPGS | Surfactant | 95.2 |
| antiviral composition | Active agent | 3.8 |

The procedure of Method A was followed.

### Method E. Multi-Component Drug Delivery System

The following ingredients were provided in the amounts indicated.

| | Weight | Weight % |
|---|---|---|
| Component | (g) | (w/w) |
| Vitamin E | 10.0 | 1.0 |
| Cremophor ELP | 580.4 | 55.9 |
| Labrasol | 89.0 | 8.6 |
| Glycerol Monooleate | 241.0 | 23.2 |
| Ethanol | 80.0 | 7.7 |
| antiviral composition | 38.5 | 3.7 |
| Total | 1038.9 | 100 |

The procedure of Method A was followed.

### Method F. Multi-Component Drug Delivery System

The following ingredients were provided in the amounts indicated an included in a capsule.

| | | Weight % |
|---|---|---|
| Component | Tradename | (w/w) |
| antiviral composition | FLAVEX | 0.6 |
| Vitamin E | Naturextrakte | 1.3 |
| Caprylocaproyl | Labrasol | 11.1 |
| polyoxyglycerides | Gattefosse 3074TPD | |
| Lauroyl | Gelucire 44/14 | 14.6 |
| polyoxyglycerides | Gattefosse 3061TPD | |
| Polyoxyl 35 Castor | Kolliphor | 72.4 |
| oil | BASF Corp. 50251534 | |
| Total | | 100 |

The procedure of Method A was followed.

### Example 4

### Preparation of Enteric Coated Capsules

### Step I: Preparation of Liquid-Filled Capsule

Hard gelatin capsules (50 counts, 00 size) were filled with a liquid composition of Example 3. These capsules were manually filled with 800 mg of the formulation and then sealed by hand with a 50% ethanol/50% water solution. The capsules were then banded by hand with 22% gelatin solution containing the following ingredients in the amounts indicated.

| | Wt. |
|---|---|
| Ingredient | (g) |
| Gelatin | 140.0 |
| Polysorbate 80 | 6.0 |
| Water | 454.0 |
| Total | 650.0 |

The gelatin solution mixed thoroughly and allowed to swell for 1-2 hours. After the swelling period, the solution was covered tightly and placed in a 55° C. oven and allowed to liquefy. Once the entire gelatin solution was liquid, the banding was performed

Using a pointed round 3/0 artist brush, the gelatin solution was painted onto the capsules. Banding kit provided by Shionogi was used. After the banding, the capsules were kept at ambient conditions for 12 hours to allow the band to cure.

### Step H1. Coating of Liquid-Filled Capsule

A coating dispersion was prepared from the ingredients listed in the table below.

| Ingredient | Wt. % | Solids % | Solids (g) | g/Batch |
|---|---|---|---|---|
| Eudragit L30D55 | 40.4 | 60.5 | 76.5 | 254.9 |
| TEC | 1.8 | 9.0 | 11.4 | 11.4 |
| AlTalc 500V | 6.1 | 30.5 | 38.5 | 38.5 |
| Water | 51.7 | na | na | 326.2 |
| Total | 100.0 | 100.0 | 126.4 | 631.0 |

If banded capsules according to Step I were used, the dispersion was applied to the capsules to a 20.0 mg/cm² coating level. The following conditions were used to coat the capsules.

| Parameters | Set-up | |
|---|---|---|
| Coating Equipment | Vector LDCS-3 | |
| Batch Size | 500 | g |
| Inlet Air Temp. | 40° | C. |
| Exhaust Air Temp. | 27-30° | C. |
| Inlet Air Volume | 20-25 | CFM |
| Pan Speed | 20 | rpm |
| Pump Speed | 9 | rpm (3.5 to 4.0 g/min) |
| Nozzle Pressure | 15 | psi |
| Nozzle diameter | 1.0 | mm |
| Distance from tablet bed* | 2-3 | in |

*Spray nozzle was set such that both the nozzle and spray path were under the flow path of inlet air.

### Example 5

### Treatment of Bovine Coronavirus Infection in an Animal

An animal presenting with bovine coronavirus infection is prescribed antiviral composition, and therapeutically relevant doses are administered to the animal according to a prescribed dosing regimen for a period of time. The animal's level of therapeutic response is determined periodically. The level of therapeutic response can be determined by determining the animal's coronavirus titer in blood or plasma. If the level of therapeutic response is too low at one dose, then the dose is escalated according to a predetermined dose escalation schedule until the desired level of therapeutic response in the animal is achieved. Treatment of the animal with antiviral composition is continued as needed and the dose or dosing regimen can be adjusted as needed until the animal reaches the desired clinical endpoint.

### Example 6

### In Vitro Evaluation of Therapeutic Antiviral Activity Against Bovine Coronavirus Infection

### Method A. Oleandrin as Sole Active

Various concentrations of Oleandrin or DMSO-matched controls were added to HRT cells either 12 hours or 24 hours after infection (MOI=0.01). The supernatant was collected 24 hours after infection from the samples previously treated at 12 hours post-infection (12-24 hrs). The supernatant was also collected 48 hours after infection from both the samples previously treated at 12 hours post-infection (12-48 hrs) and at 24 hours post-infection (24-48 hrs). Infectious BCV titers were quantified via Tissue Culture Infectious Dose (TCID50) assay. The data shown are the averages from a single representative experiment conducted in triplicate. Bar heights represent the mean and error bars represent the standard deviation. The percentage of inhibition of viral infectivity induced by Oleandrin relative to DMSO-matched controls infected cells were calculated at different time points (12-24 hrs), (12-48 hrs), and (24-48 hrs) as shown in the figures. Bar heights represent the mean and error bars represent the standard deviation.

### Method B. Oleandrin in Extract Form

Various concentrations of PBI-Oleandrin or DMSO-matched controls were added to HRT cells either 12 hours or 24 hours after infection (MOI=0.01). The supernatant was collected 24 hours after infection from the samples previously treated at 12 hours post-infection (12-24 hrs). The supernatant was also collected 48 hours after infection from both the samples previously treated at 12 hours post-infection (12-48 hrs) and at 24 hours post-infection (24-48 hrs). Infectious BCV titers were quantified via Tissue Culture Infectious Dose (TCID50) assay. The data shown are the averages from a single representative experiment conducted in triplicate. Bar heights represent the mean and error bars represent the standard deviation. The percentage of inhibition of viral infectivity induced by PBI-Oleandrin relative to DMSO-matched controls infected cells were calculated at different time points (12-24 hrs), (12-48 hrs), and (24-48 hrs) as shown in the figures. Bar heights represent the mean and error bars represent the standard deviation.

### Example 7

### Preparation of a Tablet Comprising Antiviral Composition

An initial tabletting mixture of 3% Syloid 244FP and 97% microcrystalline cellulose (MCC) was mixed. Then, an existing batch of composition prepared according to Example 3 was incorporated into the Syloid/MCC mixture via wet granulation. This mixture is labeled “Initial Tabletting Mixture) in the table below. Additional MCC was added extra-granularly to increase compressibility. This addition to the Initial Tabletting Mixture was labeled as “Extra-granular Addition.” The resultant mixture from the extra-granular addition was the same composition as the “Final Tabletting Mixture.”

| Component | Weight (g) | Weight % (w/w) |
|---|---|---|
| Initial Tabletting Mixture | | |
| Microcrystalline cellulose | 48.5 | 74.2 |
| Colloidal Silicon | 1.5 | 2.3 |
| Dioxide/Syloid 244FP | | |
| Formulation from Ex. 3 | 15.351 | 23.5 |
| Total | 65.351 | 100.0 |

Extragranular addition

| Component | Weight (g) | Weight % (w/w) |
|---|---|---|
| Initial Tabulating Mixture | 2.5 | 50.0 |
| Microcrystalline cellulose | 2.5 | 50.0 |
| Total | 5 | 100.0 |

Final Tabletting Mixture:

Abbreviated

| Component | Weight (g) | Weight % (w/w) |
|---|---|---|
| Microcrystalline cellulose | 4.36 | 87.11 |
| Colloidal Silicon | 0.06 | 1.15 |
| Dioxide/Syloid 244FP | | |
| Formulation from Ex. 3 | 0.59 | 11.75 |
| Total | 5.00 | 100 |

Final Tabletting Mixture

Detailed

| Component | Weight (g) | Weight % (w/w) |
|---|---|---|
| Microcrystalline cellulose | 4.36 | 87.11 |
| Colloidal Silicon | 0.06 | 1.15 |
| Dioxide/Syloid 244FP | | |
| Vitamin E | 0.01 | 0.11 |
| Cremophor ELP | 0.33 | 6.56 |
| Labrasol | 0.05 | 1.01 |
| Glycerol Monooleate | 0.14 | 2.72 |
| Ethanol | 0.05 | 0.90 |
| SCF extract | 0.02 | 0.44 |
| Total | 5.00 | 100.00 |

Syloid 244FP is a colloidal silicon dioxide manufactured by Grace Davison. Colloidal silicon dioxide is commonly used to provide several functions, such as an adsorbant, glidant, and tablet disintegrant. Syloid 244FP was chosen for its ability to adsorb 3 times its weight in oil and for its 5.5 micron particle size.

### Example 8

### HPLC Analysis of Solutions Containing Oleandrin

Samples (oleandrin standard, SCF extract and hot-water extract) were analyzed on HPLC (Waters) using the following conditions: Symmetry C18 column (5.0 μm, 150×4.6 mm I.D.; Waters); Mobile phase of MeOH:water=54:46 (v/v) and flow rate at 1.0 ml/min. Detection wavelength was set at 217 nm. The samples were prepared by dissolving the compound or extract in a fixed amount of HPLC solvent to achieve an approximate target concentration of oleandrin. The retention time of oleandrin can be determined by using an internal standard. The concentration of oleandrin can be determined/calibrated by developing a signal response curve using the internal standard.

### Example 9

### Preparation of Veterinary Pharmaceutical Composition

A pharmaceutical composition of the invention can be prepared any of the following methods. Mixing can be done under wet or dry conditions. The pharmaceutical composition can be compacted, dried or both during preparation. The pharmaceutical composition can be portioned into dosage forms.

### Method A.

At least one pharmaceutical excipient is mixed with at least one antiviral compound disclosed herein.

### Method B.

At least one pharmaceutical excipient is mixed with at least two antiviral compounds disclosed herein.

### Method C.

At least one pharmaceutical excipient is mixed with at least one cardiac glycosides disclosed herein.

### Method D.

At least one pharmaceutical excipient is mixed with at least two triterpenes disclosed herein.

### Method E.

At least one pharmaceutical excipient is mixed with at least one cardiac glycoside disclosed herein and at least two triterpenes disclosed herein.

### Method D.

At least one pharmaceutical excipient is mixed with at least three triterpenes disclosed herein.

### Example 10

### Preparation of Triterpene Mixtures

The following compositions were made by mixing the specified triterpenes in the approximate molar ratios indicated.

| | Triterpene (Approximate Relative Molar Content) | | |
|---|---|---|---|
| | Oleanolic | Ursolic | Betulinic |
| Composition | acid (O) | acid (U) | acid (B) |
| I (A-C) | 3 | 2.2 | 1 |
| II (A-C) | 7.8 | 7.4 | 1 |
| III (A-C) | 10 | 1 | 1 |
| IV (A-C) | 1 | 10 | 1 |
| V (A-C) | 1 | 1 | 10 |
| VI (A-C) | 1 | 1 | 0 |
| VII (A-C) | 1 | 1 | 1 |
| VIII (A-C) | 10 | 1 | 0 |
| IX (A-C) | 1 | 10 | 0 |

For each composition, three different respective solutions were made, whereby the total concentration of triterpenes in each solution was approximately 9 μM, 18 μM, or 36 μM.

| Composition | Triterpene (Approximate Content of Each, μM) | | |
|---|---|---|---|
| (total triterpene | Oleanolic | Ursolic | Betulinic |
| content, μM) | acid (O) | acid (U) | acid (B) |
| I-A (36) | 17.4 | 12.8 | 5.8 |
| I-B (18) | 8.7 | 6.4 | 2.9 |
| I-C (9) | 4.4 | 3.2 | 1.5 |
| II-A (36) | 17.3 | 16.4 | 2.2 |
| II-B (18) | 8.7 | 8.2 | 1.1 |
| II-C (9) | 4.3 | 4.1 | 0.6 |
| III-A (36) | 30 | 3 | 3 |
| III-B (18) | 15 | 1.5 | 1.5 |
| III-C (9) | 7.5 | 0.75 | 0.75 |
| IV-A (36) | 3 | 30 | 3 |
| IV-B (18) | 1.5 | 15 | 1.5 |
| IV-C (9) | 0.75 | 7.5 | 0.75 |
| V-A (36) | 3 | 3 | 30 |
| V-B (18) | 1.5 | 1.5 | 15 |
| V-C (9) | 0.75 | 0.75 | 7.5 |
| VI-A (36) | 18 | 18 | 0 |
| VI-B (18) | 9 | 9 | 0 |
| VI-C (9) | 4.5 | 4.5 | 0 |
| VII-A (36) | 12 | 12 | 12 |
| VII-B (18) | 6 | 6 | 6 |
| VII-C (9) | 3 | 3 | 3 |
| VIII-A (36) | 32.7 | 3.3 | 0 |
| VIII-B (18) | 16.35 | 1.65 | 0 |
| VIII-C (9) | 8.2 | 0.8 | 0 |
| IX-A (36) | 3.3 | 32.7 | 0 |
| IX-B (18) | 1.65 | 16.35 | 0 |
| IX-C (9) | 0.8 | 8.2 | 0 |

### Example 11

### Preparation of Antiviral Compositions

Antiviral compositions can be prepared by mixing the individual triterpene components thereof to form a mixture. The triterpene mixtures prepared above that provided acceptable antiviral activity were formulated into antiviral compositions.

Antiviral Composition with Oleanolic Acid and Ursolic Acid

Known amounts of oleanolic acid and ursolic acid were mixed according to a predetermined molar ratio of the components as defined herein. The components were mixed in solid form or were mixed in solvent(s), e.g. methanol, ethanol, chloroform, acetone, propanol, dimethyl sulfoxide (DMSO), dimethylformamide (DMF), dimethylacetamide (DMAC), N-methylpyrrolidone (NMP), water or mixtures thereof. The resultant mixture contained the components in the relative molar ratios as described herein.

For a pharmaceutically acceptable antiviral composition, at least one pharmaceutically acceptable excipient was mixed in with the pharmacologically active agents. An antiviral composition is formulated for administration to a mammal.

Antiviral Composition with Oleanolic Acid and Betulinic Acid

Known amounts of oleanolic acid and betulinic acid were mixed according to a predetermined molar ratio of the components as defined herein. The components were mixed in solid form or were mixed in solvent(s), e.g. methanol, ethanol, chloroform, acetone, propanol, dimethyl sulfoxide (DMSO), dimethylformamide (DMF), dimethylacetamide (DMAC), N-methylpyrrolidone (NMP), water or mixtures thereof. The resultant mixture contained the components in the relative molar ratios as described herein.

For a pharmaceutically acceptable antiviral composition, at least one pharmaceutically acceptable excipient was mixed in with the pharmacologically active agents. An antiviral composition is formulated for administration to a mammal.

Antiviral Composition with Oleanolic Acid, Ursolic Acid, and Betulinic Acid

Known amounts of oleanolic acid, ursolic acid and betulinic acid were mixed according to a predetermined molar ratio of the components as defined herein. The components were mixed in solid form or were mixed in solvent(s), e.g. methanol, ethanol, chloroform, acetone, propanol, dimethyl sulfoxide (DMSO), dimethylformamide (DMF), dimethylacetamide (DMAC), N-methylpyrrolidone (NMP), water or mixtures thereof. The resultant mixture contained the components in the relative molar ratios as described herein.

For a pharmaceutically acceptable antiviral composition, at least one pharmaceutically acceptable excipient was mixed in with the pharmacologically active agents. An antiviral composition is formulated for administration to a mammal.

Antiviral Composition with Oleadrin, Oleanolic Acid, Ursolic Acid, and Betulinic Acid

Known amounts of oleandrin oleanolic acid, ursolic acid and betulinic acid were mixed according to a predetermined molar ratio of the components as defined herein. The components were mixed in solid form or were mixed in solvent(s), e.g. methanol, ethanol, chloroform, acetone, propanol, dimethyl sulfoxide (DMSO), dimethylformamide (DMF), dimethylacetamide (DMAC), N-methylpyrrolidone (NMP), water or mixtures thereof. The resultant mixture contained the components in the relative molar ratios as described herein.

For a pharmaceutically acceptable antiviral composition, at least one pharmaceutically acceptable excipient was mixed in with the pharmacologically active agents. An antiviral composition is formulated for administration to a mammal.

### Example 12

### Treatment of Flavivirus Infection in an Animal

Exemplary Flavivirus infections include Yellow Fever, Dengue Fever, Japanese Encephalitis, West Nile Viruses, Zikavirus, Tick-borne Encephalitis, Kyasanur Forest Disease, Alkhurma Disease, Chikungunya virus, Omsk Hemorrhagic Fever, Powassan virus infection.

### Method A. Antiviral Composition Therapy

An animal presenting with Flavivirus infection is prescribed antiviral composition, and therapeutically relevant doses are administered to the animal according to a prescribed dosing regimen for a period of time. The animal's level of therapeutic response is determined periodically. The level of therapeutic response can be determined by determining the animal's Flavivirus titre in blood or plasma. If the level of therapeutic response is too low at one dose, then the dose is escalated according to a predetermined dose escalation schedule until the desired level of therapeutic response in the animal is achieved. Treatment of the animal with antiviral composition is continued as needed and the dose or dosing regimen can be adjusted as needed until the animal reaches the desired clinical endpoint.

Method B. Combination Therapy: Antiviral Composition with Another Agent

Method A, above, is followed except that the animal is prescribed and administered one or more other therapeutic agents for the treatment of Flavivirus infection or symptoms thereof. Then one or more other therapeutic agents can be administered before, after or with the antiviral composition. Dose escalation (or de-escalation) of the one or more other therapeutic agents can also be done.

### Example 13

### In Vitro Evaluation of Therapeutic Antiviral Activity Against Bovine Viral Diarrhea Virus (BVDV)

### Method A. Oleandrin as Sole Active

Various concentrations of oleandrin or DMSO-matched controls were added to MDBK cells either 12 hours or 24 hours after infection (MOI=0.01). The supernatant was collected 24 hours after infection from the samples previously treated at 12 hours post-infection (12-24 hrs). The supernatant was also collected 48 hours after infection from both the samples previously treated at 12 hours post-infection (12-48 hrs) and at 24 hours post-infection (24-48 hrs). Infectious BVDV titers were quantified via Tissue Culture Infectious Dose (TCID50) assay. A sample that had no detectable virus was scored as zero in the graph. The data shown are the averages from a single representative experiment conducted in triplicate. Bar heights represent the mean and error bars represent the standard deviation. The percentage of inhibition of viral infectivity induced by Oleandrin relative to DMSO-matched controls infected cells were calculated at different time points (12-24 hrs), (12-48 hrs), and (24-48 hrs) as shown in the figures. Bar heights represent the mean and error bars represent the standard deviation.

### Method B. Oleandrin in Extract

Various concentrations of PBI-Oleandrin or DMSO-matched controls were added to MDBK cells either 12 hours or 24 hours after infection (MOI=0.01). The supernatant was collected 24 hours after infection from the samples previously treated at 12 hours post-infection (12-24 hrs). The supernatant was also collected 48 hours after infection from both the samples previously treated at 12 hours post-infection (12-48 hrs) and at 24 hours post-infection (24-48 hrs). Infectious BVDV titers were quantified via Tissue Culture Infectious Dose (TCID50) assay. A sample that had no detectable virus was scored as zero in the graph. The data shown are the averages from a single representative experiment conducted in triplicate. Bar heights represent the mean and error bars represent the standard deviation. The percentage of inhibition of viral infectivity induced by PBI-Oleandrin relative to DMSO-matched controls infected cells were calculated at different time points (12-24 hrs), (12-48 hrs), and (24-48 hrs) as shown in the figures. Bar heights represent the mean and error bars represent the standard deviation.

### Example 14

### In Vitro Evaluation of Therapeutic Antiviral Activity Against Porcine Reproductive and Respiratory Syndrome Virus (PRRSV)

### Method A. Oleandrin as Sole Active

Various concentrations of oleandrin or DMSO-matched controls were added to MARC 145 cells either 12 hours or 24 hours after infection (MOI=0.01). The supernatant was collected 24 hours after infection from the samples previously treated at 12 hours post-infection (12-24 hrs). The supernatant was also collected 48 hours after infection from both the samples previously treated at 12 hours post-infection (12-48 hrs) and at 24 hours post-infection (24-48 hrs). Infectious PRRSV titers were quantified via Tissue Culture Infectious Dose (TCID50) assay. The data shown are the averages from a single representative experiment conducted in triplicate. Bar heights represent the mean and error bars represent the standard deviation. The percentage of inhibition of viral infectivity induced by oleandrin relative to DMSO-matched controls infected cells were calculated at different time points (12-24 hrs), (12-48 hrs), and (24-48 hrs) as shown in the figures. Bar heights represent the mean and error bars represent the standard deviation.

### Method B. Oleandrin in Extract

Various concentrations of PBI-oleandrin or DMSO-matched controls were added to MARC 145 cells either 12 hours or 24 hours after infection (MOI=0.01). The supernatant was collected 24 hours after infection from the samples previously treated at 12 hours post-infection (12-24 hrs). The supernatant was also collected 48 hours after infection from both the samples previously treated at 12 hours post-infection (12-48 hrs) and at 24 hours post-infection (24-48 hrs). Infectious PRRSV titers were quantified via Tissue Culture Infectious Dose (TCID50) assay. The data shown are the averages from a single representative experiment conducted in triplicate. Bar heights represent the mean and error bars represent the standard deviation. The percentage of inhibition of viral infectivity induced by PBI-oleandrin relative to DMSO-matched controls infected cells were calculated at different time points (12-24 hrs), (12-48 hrs), and (24-48 hrs) as shown in the figures. Bar heights represent the mean and error bars represent the standard deviation.

### Example 15

### In Vitro Evaluation of Therapeutic Antiviral Activity Against Bovine Respiratory Syncytial Virus (BRSV)

### Method A. Oleandrin as Sole Active

The therapeutic assay was completed according to the established protocol; with the exception that 500 μL of maintenance media containing 1×104 TCID50 per well was added to the wells instead of 100 μL, to ensure adequate coverage of the cells for the incubation period. BT cells were plated 48 hours prior to the assay. At the time of the assay, the media was removed and replaced with virus maintenance media containing virus at an MOI of 0.01 in each well. A separate set of plates was incubated for either 12 or 24 hours. At each timepoint (12 or 24 hours), plates were washed gently with DPBS and then 2 ml of virus maintenance medium containing the desired concentrations of Oleandrin or PBI-05204 dissolved in DMSO, or matched concentrations of DMSO-only was added to each well. Oleandrin, PBI, and DMSO dilutions were made 5-hours prior to the 12-hour treatment and stored protected from light at 4° C. (prepared at 4 pm and used at 9 pm). Fresh Oleandrin, PBI, and DMSO dilutions were made prior to the 24-hour treatment. Samples were removed at 24 and 48 hours after the 12-hour virus inoculation, and at 48 hours after the 24-hour virus inoculation and aliquoted into two cryovials. Virus isolations were performed immediately on samples collected at each time point and the aliquots were then frozen at −80° C. Samples were submitted to the Molecular Diagnostics Section at ADRDL at South Dakota State University for qRT-qPCR.

### Method B. Oleandrin in Extract

Method A was repeated with the exception that an extract containing oleandrin was used in place of pure oleandrin. The amount of extract used was normalized according to its oleandrin content, which was used in the assay in amounts equivalent to pure oleandrin. Various concentrations of PBI-Oleandrin or DMSO-matched controls were added to BT cells either 12 hours or 24 hours after infection (MOI=0.01). The supernatant was collected 24 hours after infection from the samples previously treated at 12 hours post-infection (12-24 hrs). The supernatant was also collected 48 hours after infection from both the samples previously treated at 12 hours post-infection (12-48 hrs) and at 24 hours post-infection (24-48 hrs). Infectious BRSV titers were quantified via Tissue Culture Infectious Dose (TCID50) assay. A sample that had no detectable virus was scored as zero in the graph. The data shown are the averages from a single representative experiment conducted in triplicate. Bar heights represent the mean and error bars represent the standard deviation. The percentage of inhibition of viral infectivity induced by PBI-Oleandrin relative to DMSO-matched controls infected cells were calculated at different time points (12-24 hrs), (12-48 hrs), and (24-48 hrs) as shown in the figures. Bar heights represent the mean and error bars represent the standard deviation.

### Example 16

### Statistical Analysis

The statistical significance of experimental data sets was determined using unpaired two-tailed Student's t-tests (alpha=0.05) and calculated P-values using the Shapiro-Wilk normality test and Graphpad Prism 7.03 software. The P-values were defined as: 0.1234 (ns), 0.0332 (*), 0.0021 (**), 0.0002 (***), <0.0001 (****). Unless otherwise noted, error bars represent the SEM from at least three independent experiments.

### Example 17

### Treatment of Viral Infection in a Cow

RSV, BVDV or BCV infection in a cow is treated by administering plural doses of oleandrin containing composition. The composition can be a veterinary pharmaceutical composition, a feed, or a liquid. It may be administered orally, by injection, by implantation, or by other means known to be suitable for administration of compounds to cattle. The amount of oleandrin administered to the cow should be such that the corresponding plasma concentration of oleandrin in the cow is not more than 1 ng/mL.

### Example 18

### In Vitro Evaluation of Oleandrin Toxicity Against Cells

The purpose of this assay was to determine the relative potential toxicity of oleandrin against various cells in vitro.

Oleandrin (PhytoLab, Vestenbergsgreuth, Germany) was dissolved at a concentration of 1 mg/ml in DMSO. The desired concentration range to be used in cytotoxicity testing is 0.005-1 ug/ml in 0.0005-0.1% wt in DMSO, respectively. Lactate dehydrogenase release assay (LDH assay) was be used to determine the cytotoxic effect of different concentration of Oleandrin on different cell cultures. LDH is a cytosolic enzyme that is released only from damaged cells (due to increased membrane permeability) to the outside medium that will convert the lactate in the medium into pyruvate in a coupled reaction that includes the reduction of NAD+ into NADH, the latter is oxidized back to NAD+ in the presence of Diaphorase in the LDH kit mix that leads to the reduction of water soluble tetrazolium (INT), that is also in the kit mix, into red Formazon product that can be read by an ELISA reader at a wave length of 490 nm. The cytotoxic effect of 8 different concentrations of Oleandrin (0.005-1 ug/ml) on 3 different cell line was tested on BT (Bos taurus turbinate), MDBK (Bos taurus kidney) and MARC 145 (monkey kidney) cells in triplicates at 2 different times points 24 and 48 hours post treatment.

This was achieved by preparing different concentration of cells in 100 μl volume staring at 1000 cell/100 μl to 20,000 cell/100 μl in a 2-fold serial dilution manner. This serial dilution was done in triplicate for 2 sets of cells, one set used as cell control, that would report the spontaneous LDH release, and the other set was treated with cell lysis buffer to report the maximum LDH release. After performing the test according to the manufacturer instructions, the average OD value reading of the triplicate for each dilution in each set was taken and plotted against number of cells. The best cell seeding capacity/100 μl volume was the one that achieved maximum LDH release of 1.6-2 and spontaneous LDH release of less than 0.5. after 30 min of incubation with the kit mix (manufacturer instructions) at both time points. The optimal number of cells/well in 100 μL of growth medium (as determined in preliminary experiments) was plated in triplicate in wells in a 96-well tissue culture plate. Cells were incubated overnight at 37° C. with the appropriate level of CO₂. The following day the growth medium was removed by washing the cells twice with PBS. The growth media was replaced with 100 μl of maintenance media containing either 1-0.005 ug/ml oleandrin, 0.1-0.0005% DMSO without drug, or untreated media to serve as controls for the maximum and spontaneous release 250 of LDH. All treatments were added to triplicate wells, and the plate was returned to the 37° C./5% CO₂ incubator for 24-48 hours. At either 24 or 48 hours post-treatment, the plate was removed from the incubator and the LDH released into the supernatant was assessed by CyQUANT LDH toxicity assay (Thermofisher, Eugene, OR) according to the manufacturer's directions. Absorbance is measured at 490 nm and 680 nm using Spectramax i3x. The corrected OD value of the max LDH release control should be around 1.6-2 and that for the spontaneous LDH release control should be below 0.5.

To determine the cytotoxicity of Oleandrin and DMSO, the following equation was applied to the corrected OD value of each concentration:

$$\% cytotoxicity of individual concentration = corrected OD value of ( ( {Treatment}^{*} - Spontaneous LDH release ) / ( Maximum LDH release - Spontaneous LDH release ) ) \times 100$$

To determine the safe dosage of Oleandrin, the % cytotoxicity was maintained as less than 2% in each time points for the Oleandrin concentration and the corresponding DMSO concentration.

### Example 19

Preparation of Subcritical Fluid Extract of Nerium oleander

An improved process for the preparation of an oleandrin-containing extract was developed by employing subcritical liquid extraction rather than supercritical fluid extraction of Nerium oleander biomass.

Dried and powdered biomass was placed in an extraction chamber, which was then sealed. Carbon dioxide (about 95% wt) and alcohol (about 5% wt; methanol or ethanol) were injected into the chamber. The interior temperature and pressure of the chamber were such that the extraction medium was maintained in the subcritical liquid phase, rather than the supercritical fluid phase, for a majority or substantially all of the extraction time period: temperature in the range of about 2° C. to about 16° C. (about 7° C. to about 8° C.), and pressure in the range of about 115 to about 135 bar (about 124 bar). The extraction period was about 4 h to about 12 h (about 6 to about 10 h). The extraction milieu was then filtered and the supernatant collected. The carbon dioxide was vented from the supernatant, and the resulting crude extract was diluted into ethanol (about 9 parts ethanol:about 1 part extract) and frozen at about −50° C. for at least 12 h. The solution was thawed and filtered (100 micron pore size filter). The filtrate was concentrated to about 10% of its original volume and then sterile filtered (0.2 micron pore size filter). The concentrated extract was then diluted with 50% aqueous ethanol to a concentration of about 1.5 mg of extract per mL of solution.

The resulting subcritical liquid (SbCL) extract comprised oleandrin and one or more other compounds extractable from Nerium oleander, said one or more other compounds being as defined herein.

### Example 20

Preparation of Ethanolic Extract of Nerium oleander

The purpose of this was to prepare an ethanolic extract by extraction of Nerium oleander biomass with aqueous ethanol.

Ground dried leaves were repeatedly treated with aqueous ethanol (90-95% v/v ethanol; 10-5% v/v water). In some cases, the temperature was above ambient. The combined ethanolic supernatants were combined and filtered and then concentrated by evaporation in vacuo to reduce the amount of ethanol and water therein and provide crude ethanolic extract comprising about 25 mg of oleandrin/mL of extract (which has about 50% v/v ethanol content).

### Example 21

Preparation of Dosage Form Comprising a Combination of Extracts of Nerium oleander

The purpose of this was to prepare a dosage form according to Example 32 except that a portion (1 wt %) of the ethanolic extract of Example 36 is combined with a portion (1 wt %) of the SbCL extract of Example 33, medium chain triglyceride (95 wt %), and flavoring agent (3 wt %).

### Example 22

### In Vitro Evaluation of Prophylactic Antiviral Activity Against Bovine Coronavirus Infection

### Method A. Oleandrin as Sole Active

Various concentrations of Oleandrin or DMSO-matched controls were added to HRT cells 30 minutes before infection (MOI=0.01 based on TCID50) and were maintained after infection as well. The supernatant was collected (A) 24 hours and (B) 48 hours after infection. Infectious BCV titers were quantified via Tissue Culture Infectious Dose (TCID50) assay (A and B). A sample that had no detectable virus was scored as zero in the graph. The data shown are the averages from a single representative experiment conducted in triplicate. Bar heights represent the mean and error bars represent the standard deviation. The percentage of inhibition of viral infectivity induced by Oleandrin relative to DMSO-matched controls infected cells were calculated at 24 and 48 hours. Bar heights represent the mean and error bars represent the standard deviation.

### Method B. Oleandrin in Extract Form

Various concentrations of PBI-Oleandrin or DMSO-matched controls were added to HRT cells 30 minutes before infection (MOI=0.01 based on TCID50) and were maintained after infection as well. The supernatant was collected 24 hours and 48 hours after infection. Infectious BCV titers were quantified via Tissue Culture Infectious Dose (TCID50) assay. A sample that had no detectable virus was scored as zero in the graph. The data shown are the averages from a single representative experiment conducted in triplicate. Bar heights represent the mean and error bars represent the standard deviation. The percentage of inhibition of viral infectivity induced by PBI-Oleandrin relative to DMSO-matched controls infected cells were calculated at 24 and 48 hours. Bar heights represent the mean and error bars represent the standard deviation.

### Example 23

### In Vitro Evaluation of Prophylactic Antiviral Activity Against BVDV

### Method A. Oleandrin as Sole Active

Various concentrations of oleandrin or DMSO-matched controls were added to MDBK cells 30 minutes before infection (MOI=0.01 based on TCID50) and were maintained after infection as well. The supernatant was collected 24 hours and 48 hours after infection. Infectious BVDV titers were quantified via Tissue Culture Infectious Dose (TCID50) assay. A sample that had no detectable virus was scored as zero in the graph. The data shown are the averages from a single representative experiment conducted in triplicate. Bar heights represent the mean and error bars represent the standard deviation. The percentage of inhibition of viral infectivity induced by Oleandrin relative to DMSO-matched controls infected cells were calculated at 24 and 48 hours. Bar heights represent the mean and error bars represent the standard deviation.

### Method B. Oleandrin in Extract Form

Various concentrations of PBI-oleandrin or DMSO-matched controls were added to MDBK cells 30 minutes before infection (MOI=0.01 based on TCID50) and were maintained after infection as well. The supernatant was collected 24 hours and 48 hours after infection. Infectious BVDV titers were quantified via Tissue Culture Infectious Dose (TCID50) assay. A sample that had no detectable virus was scored as zero in the graph. The data shown are the averages from a single representative experiment conducted in triplicate. Bar heights represent the mean and error bars represent the standard deviation. The percentage of inhibition of viral infectivity induced by PBI-Oleandrin relative to DMSO-matched controls infected cells were calculated at 24 and 48 hours. Bar heights represent the mean and error bars represent the standard deviation.

### Example 24

### In Vitro Evaluation of Prophylactic Antiviral Activity Against PRRSV Infection

### Method A. Oleandrin as Sole Active

Various concentrations of oleandrin or DMSO-matched controls were added to MARC 145 cells 30 minutes before infection (MOI=0.01 based on TCID50) and were maintained after infection as well. The supernatant was collected 24 hours and 48 hours after infection. Infectious PRRSV titers were quantified via Tissue Culture Infectious Dose (TCID50) assay. A sample that had no detectable virus was scored as zero in the graph. The data shown are the averages from a single representative experiment conducted in triplicate. Bar heights represent the mean and error bars represent the standard deviation. The percentage of inhibition of viral infectivity induced by Oleandrin relative to DMSO-matched controls infected cells were calculated at 24 and 48 hours respectively. Bar heights represent the mean and error bars represent the standard deviation.

### Method B. Oleandrin in Extract Form

Various concentrations of PBI-oleandrin or DMSO-matched controls were added to MARC 145 cells 30 minutes before infection (MOI=0.01 based on TCID50) and were maintained after infection as well. The supernatant was collected 24 hours and 48 hours after infection. Infectious PRRSV titers were quantified via Tissue Culture Infectious Dose (TCID50) assay. A sample that had no detectable virus was scored as zero in the graph. The data shown are the averages from a single representative experiment conducted in triplicate. Bar heights represent the mean and error bars represent the standard deviation. The percentage of inhibition of viral infectivity induced by PBI-Oleandrin relative to DMSO-matched controls infected cells were calculated at 24 and 48 hours respectively. Bar heights represent the mean and error bars represent the standard deviation.

### Example 25

### In Vitro Evaluation of Prophylactic Antiviral Activity Against BRSV Infection

### Method A. Oleandrin as Sole Active

BT cells were plated 48 hours prior to the assay. At the time the of the assay, the media was removed from each well and replaced with media containing the desired concentrations of Oleandrin dissolved in DMSO, or matched concentrations of DMSO-only. Oleandrin, and DMSO dilutions were made fresh prior to the assay. Plates were incubated with product for 30 minutes, then BRSV virus at an MOI of 0.01 was added to each well. Virus was incubated on the plates for 1 hour and then removed. Plates were washed gently with DPBS and 2 ml of virus maintenance medium containing Oleandrin, PBI, or DMSO-only was added to each well. Samples were removed at each time point (24 and 48 hr) and aliquoted into two cryovials. Virus isolations were performed immediately on samples collected at each time point and the aliquots were then frozen at −80° C. Samples were submitted to the Molecular Diagnostics Section at ADRDL at South Dakota State University for RT-qPCR.

### Method B. Oleandrin in Extract Form

Method A was repeated with the exception that an extract containing oleandrin was used in place of pure oleandrin. The amount of extract used was normalized according to its oleandrin content, which was used in the assay in amounts equivalent to pure oleandrin. Various concentrations of PBI-oleandrin or DMSO-matched controls were added to BT cells 30 minutes before infection (MOI=0.01 based on TCID50) and were maintained after infection as well. The supernatant was collected 24 hours and 48 hours after infection. Infectious BRSV titers were quantified via Tissue Culture Infectious Dose (TCID50) assay. A sample that had no detectable virus was scored as zero in the graph. The data shown are the averages from a single representative experiment conducted in triplicate. Bar heights represent the mean and error bars represent the standard deviation. The percentage of inhibition of viral infectivity induced by PBI-Oleandrin relative to DMSO-matched controls infected cells were calculated. Bar heights represent the mean and error bars represent the standard deviation.

### Example 26

### In Vitro Evaluation of Antiviral Activity Against Bovine Herpesvirus Type-1

The method of Babiuk et al. (“Effect of bovine alpha1 interferon on bovine herpesvirus type-1-induced respiratory disease” in J. Gen. Virol (1985), 66, 2383-2394) is followed except that solutions containing different concentrations of oleandrin or digoxin are used in place of the interferon.

### Example 27

### In Vitro Evaluation of Antiviral Activity Against Porcine Circovirus Type-1

The method of Meerts et al. (“Correlation between type of adaptive immune response against porcine circovirus type 2 and level of virus replication” in Viral Immun. (2005), 18, 333-341) is followed except that solutions containing different concentrations of oleandrin or digoxin are used in place of the cyclosporin A.

### Example 28

### In Vitro Evaluation of Antiviral Activity Against Foot and Mouth Disease Virus

The method of Airaksinen et al. (“Curing of foot and mouth disease virus from persistently infected cells with ribavirin involves enhanced mutagenesis” in Virology (2003), 311, 339-349) is followed except that solutions containing different concentrations of oleandrin or digoxin are used in place of the ribavirin.

### Example 29

### In Vitro Evaluation of Antiviral Activity Against African Swine Fever Virus

The method of Arabyan et al. (“Antiviral agents against African swine fever virus” in Virus Res. (2019), 270, 197669) is followed except that solutions containing different concentrations of oleandrin or digoxin are used in place of other antiviral agent(s).

### Example 30

### In Vitro Evaluation of Antiviral Activity Against African Horse Sickness Virus

The method of Goris et al. (“Potential of antiviral therapy and prophylaxis for controlling RNA viral infections of livestock” in Antiviral Res. (2008), 78(1), 170-178) is followed except that solutions containing different concentrations of oleandrin or digoxin are used in place of other antiviral agent(s).

### Example 31

### In Vitro Evaluation of Antiviral Activity Against Sheeppox Virus and Lumpy Skin Disease Virus

The method of Toker et al. (“Inhibition of bovine and ovine capripoxviruses (Lumpy skin disease virus and sheeppox virus) by ivermectin occurs at different stages of propagation in vitro” in Virus Res. (2022), 310, 198671) is followed except that solutions containing different concentrations of oleandrin or digoxin are used in place of ivermectin.

### Example 32

### In Vitro Evaluation of Antiviral Activity Against Swine Vesicular Disease Virus

The method of de Leon et al. (“Inhibition of porcine viruses by different cell-targeted antiviral drugs” in Front. Microbiol. (2019), 10, 1853; doi.org/10.3389/fmicb.2019.01853) is followed except that solutions containing different concentrations of oleandrin or digoxin are used in place of other antiviral agent(s).

### Example 33

### In Vitro Evaluation of Antiviral Activity Against Avian Infectious Bronchitis Virus

The method of Lelesius et al. (“In vitro antiviral activity of fifteen plant extracts against avian infectious bronchitis virus” in BMC Vet. Res. (2019), 15, 178) is followed except that solutions containing different concentrations of oleandrin or digoxin are used in place of the extracts.

### Example 34

### In Vitro Evaluation of Antiviral Activity Against Infectious Bursal Disease Virus and Newcastle Disease

The method of Mo et al. (“The in vivo and in vitro effects of chicken interferon alpha on infectious bursal disease virus and Newcastle disease virus infection” in Avian Dis. (2001), 45, 389-399) is followed except that solutions containing different concentrations of oleandrin or digoxin are used in place of the interferon.

### Example 35

### In Vitro Evaluation of Antiviral Activity Against Avian Influenza Virus

The method of Beigel et al. (“Current and future antiviral therapy of severe seasonal and avian influenza” in Antiviral Res. (2008), 78(1), 91-102) is followed except that solutions containing different concentrations of oleandrin or digoxin are used in place of other antiviral agent(s).

### Example 36

### In Vitro Evaluation of Antiviral Activity Against Marek's Disease Virus

The method of Sun et al. (“Screening compounds of Chinese medicinal herbs anti-Marek's disease virus” in Pharm. Biol. (2014), 52(7), 841-847) is followed except that solutions containing different concentrations of oleandrin or digoxin are used in place of other herb extracts.

### Example 37

### In Vitro Evaluation of Antiviral Activity Against Poult Enteritis Mortality Syndrome in Turkeys

The method of Shehata et al. (“Poult enteritis and mortality syndrome in turkey poults: causes, diagnosis and preventive measures” in Animals (2021), 11, 2063) is followed except that solutions containing different concentrations of oleandrin or digoxin are used in place of other agent(s).

### Example 38

### In Vitro Evaluation of Antiviral Activity Against Canine Distemper Virus

The method of Fabiana et al. (“Antiviral efficacy of EICAR against canine distemper virus (CDV) in vitro” in Res. Vet. Sci. (2010), 339-344) is followed except that solutions containing different concentrations of oleandrin or digoxin are used in place of EICAR.

### Example 39

### In Vitro Evaluation of Antiviral Activity Against Canine Influenza Virus

The method of Ashton et al. (“In vitro susceptibility of canine influenza A (H3N8) virus to nitazoxanide and tizoxanide” in Vet. Med. Inter. (2010), 891010) is followed except that solutions containing different concentrations of oleandrin or digoxin are used in place of nitazoxanide and tizoxanide.

### Example 40

### In Vitro Evaluation of Antiviral Activity Against Feline Herpes Virus

The method of Thomasy et al. (“A review of antiviral drugs and other compounds with activity against feline herpesvirus-1” in Vet. Ophthal. (2016), 19 (Suppl. 1), 119-130) is followed except that solutions containing different concentrations of oleandrin or digoxin are used in place of other agent(s).

### Example 41

### In Vitro Evaluation of Antiviral Activity Against Type I Feline Infectious Peritonitis Virus

The method of Doki et al. (“In vivo antiviral effects of U18666A against type I feline infectious peritonitis virus” in Pathogens (2020), 9, 67) is followed except that solutions containing different concentrations of oleandrin or digoxin are used in place of U18666A.

### Example 42

### In Vitro Evaluation of Antiviral Activity Against Feline Rotavirus

The method of Tellez et al. (“In vitro antiviral activity against rotavirus and astrovirus infection exerted by substances obtained from Achyroline bogotensis (Kunth) DC. (compositae” in BMC Compl. Altern. Med. (2015), 15, 428) is followed except that solutions containing different concentrations of oleandrin or digoxin are used in place of other extract.

### Example 43

### In Vitro Evaluation of Antiviral Activity Against Porcine Deltacoronavirus

The method of Zhai et al. (“Antiviral effect of lithium chloride and diammonium glycyrrhizinate on porcine deltacoronavirus in vitro” in Pathogens (2019), 8, 144) is followed except that solutions containing different concentrations of oleandrin or digoxin are used in place of lithium chloride and diammonium glycyrrhizinate.

### Example 44

### In Vitro Evaluation of Antiviral Activity Against Swine Influenza Virus

Cells are plated in 12 well plates at a concentration of approximately 5×10₅ cells/well. Oleandrin/extract/DMSO concentrations are tested in triplicate. The cells are incubated for 48 hours until confluent.

### Prophylactic Testing

Susceptible cells are pre-treated with pure oleandrin or extract at desired concentrations, infected with the virus, then incubated for 48 hours in virus maintenance media also containing the same concentrations of oleandrin as in the pre-treatment. Samples are collected at 24 and 48 hours for subsequent TCID50 and RT-RT-qPCR determination.

Remove growth media from confluent monolayers of approximately 5×10₅ cells in 12-wells plates, washed twice with PBS and replace with 200 μL of maintenance media and the desired concentration of oleandrin dissolved in DMSO or matched DMSO-only control wells. The plates are incubated at 37° C./5% CO₂ for 30 minutes. After pre-treatment, 5×10₃ virus units in a volume of 500 μL are added to each well (MOI=0.01). This is incubated at 37° C./5% CO₂ for 1 hour. The plates are washed gently 3 times with DPBS. Then 2 mL of virus maintenance medium containing pure oleandrin in DMSO, extract, or DMSO-only are added to each well at the same pre-treatment concentration. The samples are collected from each well at 24 and 48 hours for TCID₅₀ and RT-qPCR determination. Collected samples are stored at −80° C. until testing.

### Therapeutic Testing

Susceptible cells are infected with the virus and incubated for up to 48 hours. At 12- or 24 hours post-infection, infected cells will be treated with pure oleandrin or extract at the desired concentrations in virus maintenance media. Supernatant is collected at 24 hours after infection from the samples previously treated at 12 hours post-infection and at 48 hours after infection from both the samples previously treated at 12 and 24 hours post infections for subsequent TCID50 and RT-qPCR determination.

Remove growth media from confluent monolayers of approximately 5×10₅ cells in 12-well plates and replace with 500 μL of maintenance media and the SIV virus at an MOI of 0.01 in each well. The plates are incubated at 37° C./5% CO₂ for 12- or 24-hours. The plates are washed gently 3 times with DPBS. 4. 2 mL of virus maintenance medium containing pure oleandrin in DMSO, extract or DMSO-only matched concentration as in the prophylactic treatment concentrations are added at 12- or 24 hours post-infection. Samples are collected from 12-hour post-infection oleandrin or DMSO-only treatment at 24- and 48-hours post-infection for TCID₅₀ and RT-qPCR determination. Samples are collected from 24-hour post-infection oleandrin or DMSO-only treatment at 48-hours post-infection for TCID₅₀ and RT-qPCR determination. Collected samples from each well at 24 and 48 hours for TCID₅₀ and RT-qPCR determination are stored at −80° C. until testing.

### Example 45

### Intramuscular Administration of OCC to Dogs (Safety Study)

A stock aqueous solution containing ANVIRZEL powder (300 mg; ANVIZEL contained 350-400 microg oleandrin (OLE) per g of powder) dissolved in water for injection (20 mL) was prepared (resulting concentration: 15 mg ANVIRZEL/mL; 17.5-20 microg OLE/mL).

Beagle dogs (16 male, 16 female) ranging in weight from 9.4-11.5 kg (males) and 6.5-10.7 kg (females) were selected for the study. The age of the dogs ranged from 7-11 months on day-1 of administration. Each gender was divided into four cohorts according to the dose (amount of stock solution) administered once daily (for 28 days) via i.m. injection. Water for injection was used as the control.

| | Cohort I | Cohort II | Cohort III | Control |
|---|---|---|---|---|
| ANVIRZEL | 0.375 | 0.75 | 1.5 | 0 |
| (mg/Kg/day) | | | | |
| Equivalent | 0.14 | 0.28 | 0.56 | 0 |
| OLE (microg/ | | | | |
| Kg/day | | | | |
| Volume | 0.025 | 0.05 | 0.1 | 15 |
| administered | mL/Kg/day | mL/Kg/day | mL/Kg/day | mL/day |

No mortality was observed. No drug-related undesirable side effects were observed.

### Example 46

Preparation of Methanolic Extract of Nerium oleander

The purpose of this was to prepare a methanolic extract by extraction of Nerium oleander biomass with methanol.

Ground dried leaves were repeatedly treated with methanol. In some cases, the temperature was above ambient, e.g. hot (>21° C. to refluxing). The combined methanolic supernatants were combined and filtered and then concentrated by evaporation in vacuo to substantially to dryness and provide crude methanolic extract comprising about 2-4 mg of oleandrin/g of solid extract.

### Example 47

### Administration of Oleandrin Containing Composition to Dogs (Oral) (Safety Study)

The SCF extract (PBI-05204) was formulated as detailed above into a micelle forming delivery system, i.e. a composition that forms micelles when the composition is mixed with aqueous, such as water, buffer, saliva, gastric fluid, intestinal fluid, or colonic fluid. The content of OLE in the formulated composition was typically in the range of 0.05-0.1%.

Formulated PBI-05204 composition or control vehicle were orally administered to beagle dogs at once daily doses of 0.1, 0.3, or 1.0/0.6 mg/kg oleander extract (equivalent to 2.3 μg, 6.9 μg, and 23/13.8 μg of oleandrin/kg, respectively) for 28 consecutive days followed by a 14-day recovery period. This resulted in a no-observable-adverse-effect-level (NOAEL) of 0.1 mg/kg. The adverse effects at 0.3 or 1.0/0.6 mg/kg were reversible following a 14-day recovery period.

In order to determine an approximate MTD, a dose-range finding (single dose) study of formulated PBI-05204 composition was conducted in beagle dogs. The relative drug toxicity in dogs treated with escalating oral doses was determined. One male and one female dog were provided the following escalating doses of 5, 10, and 20 mg/kg (equivalent to 115 pg, 230 pg, and 460 pg of oleandrin/Kg, respectively) followed by a 3-day washout/observation period between doses. The results in the male indicated a dose-dependent increase in abnormal clinical observations consisting of vomiting, abnormal stool, increased salivation, labored breathing, decreased activity, and death. After the female dog received 20 mg/kg of formulated PBI-05204 (equivalent to 460 pg of oleandrin/Kg), a severe arrhythmia characterized as sinus arrest with ventricular escape rhythm.

A 28-day toxicity study of formulated PBI-05204 in beagle dogs followed by a 14-day recovery period was conducted. The objective of this study was to determine the potential systemic toxicity of test article administered once daily for 28 consecutive days via oral (gavage) administration followed by a 14-day recovery period. A total of 36 dogs were dosed with test article PBI-05204 containing 37.2 mg/mL (at concentrations of 0.1, 0.3, or 1.0/0.6 mg/kg) oleander extract (equivalent to 2.3, 6.9, 23/13.8 ug oleandrin/kg, respectively) or control vehicle PBI-05204 Control Vehicle (Placebo) via oral gavage once daily for 28 consecutive days (except for Group 4 animals). There were no early deaths during the study. All animals survived until their scheduled necropsy date. Test article-related clinical findings include the following: abnormal stool primarily in Groups 3 and 4 males, thin body only in Group 4 animals with a delayed onset in females when compared to males, decreased activity in Group 4 males only, inappetence in Group 4 males only, defecation decreased in Group 4 males only, predominant post-dose vomiting in Group 4 (males>females), predominant increased salivation in Group 4 (males>females), bloodshot eyes in Group 4 males only, predominant excessive lacrimation in Group 4 animals, and ptosis in Group 4 males only. Compared to Study Day 1, Study Day 29 bodyweights for Groups 3 and 4 males were decreased. There were no apparent test article-affects on feed consumption with the exception of decreased feed intake in Group 4 males on Study Days 7 through 13. There were no apparent test article-affects on individual body temperatures with the exception of decreased body temperatures in Group 4 males on Study Day 8. Based on the measured hematology parameters, there were no apparent biological or toxicological findings. On Study Day 29, Group 4 male heart weight trends (absolute and relative) were decreased in comparison to concurrent controls. This finding appears to be test article-related. The administration of test article did not cause a discernable change in QRS duration, or QT interval, or QTcf or QTcv throughout this study. However, in dogs receiving 1.0/0.6 mg/kg/day, the heart rate was decreased. In dogs receiving 0.3 or 1.0/0.6 mg/kg/day, atrioventricular conduction was slowed and indicated by prolongation of the PR interval and the development of secondary degree atrioventricular block. Also atrial premature depolarization developed in dogs receiving over 0.3 or 1.0/0.6 mg/kg/day. The presence of these findings both before and after test article-administration on Study Week 4 suggests at least a 23 hour duration of the test article-induced changes. In conclusion, the administration of 0.1; 0.3; or 1.0/0.6 mg/kg) oleander extract, or control vehicle PBI-05204 Control Vehicle (Placebo) via oral gavage once daily for 28 consecutive days (except for Group 4 animals) followed by a 14 day recovery period resulted in a no-observable-adverse-effect-level (NOAEL) of 0.1 mg/kg (equivalent to 2.3 ug oleandrin/kg). The adverse effects at 0.3 or 1.0/0.6 mg/kg were reversible following a 14-day recovery period.

### Example 48

Administration of Nerium oleander Extract to Pigs

The following method was used to evaluate the safe administration of the MCT oil-based product (PBI-06150) described herein containing a combination of Nerium oleander subcritical extract and alcohol extract dissolved in the MCT oil to provide a concentration of about 13.5 microg of oleandrin/mL.

A nursing sow (weighing about 400 lbs or about 182 Kg) was administered product according to the following schedule and the content of oleandrin in plasma and milk were determined according to the method of Tor et al., who used HPLC-MSMS analysis.

| | Dose | | |
|---|---|---|---|
| | (microg OLE) | | |
| | (microg OLE/ | | |
| Day | Kg BW) | Action | Test result |
| 1 | 50 | Collect milk and | No OLE in |
| | | draw blood @5 h | milk or blood |
| | 0.125 | after administration | |
| 3 | 100 | Collect milk and | No OLE in |
| | | draw blood @5 h | milk or blood |
| | 0.25 | after administration | |
| 5 | 200 | Collect milk and | No OLE in |
| | | draw blood @5 h | milk or blood |
| | 0.5 | after administration | |
| 7 | 400 | Collect milk and | No OLE in |
| | | draw blood @5 h | milk or blood |
| | 1.0 | after administration | |

A weaned piglet (about 23 days of age and weighing about 12.7 lbs) was administered a sufficient amount of PBI-06150 to provide 160 microg of OLE (a dose equivalent to 27.7 microg of OLE/Kg BW). Blood samples were drawn periodically, and their content of OLE determined. The following results were obtained.

| Time after | | OLE content |
|---|---|---|
| administration (h) | Action | in blood (ng/mL) |
| 0 | Collect blood then dose | 0 |
| 1 | Collect blood | 11.4 |
| 2 | Collect blood | 10.24 |
| 4 | Collect blood | 4.18 |
| 8 | Collect blood | BLQ |
| 12 | Collect blood | BLQ |
| 24 | Collect blood | BLQ |

BLQ denotes below level of quantitation (below 2.5 ng/ml)

OLE tissue distribution data was obtained for four of the above piglets after euthanization: subjects 1 and 2 were euthanized 1 day after administration; and subjects 3 and 4 were euthanized 8 days after administration. Muscle tissue and organs were collected after euthanization and tested for OLE content by HPLC-MSMS. The following results were obtained.

| Time after | | |
|---|---|---|
| administration | OLE content in heart tissue | |
| (days) | (ng OLE/g of tissue) | |
| 1 | Subj. #1: 25.3 | Subj. #2: 17.0 |
| 8 | Subj. #3: BLQ | Subj. #4: BLQ |

| Time after | | |
|---|---|---|
| administration | OLE content in muscle tissue | |
| (days) | (ng OLE/g of tissue) | |
| 1 | Subj. #1: BLQ | Subj. #2: BLQ |
| 8 | Subj. #3: BLQ | Subj. #4: BLQ |

| Time after | | |
|---|---|---|
| administration | OLE content in kidney tissue | |
| (days) | (ng OLE/g of tissue) | |
| 1 | Subj. #1: 43.3 | Subj. #2: 65.7 |
| 8 | Subj. #3: BLQ | Subj. #4: BLQ |

| Time after | | |
|---|---|---|
| administration | OLE content in lung tissue | |
| (days) | (ng OLE/g of tissue) | |
| 1 | Subj. #1: BLQ | Subj. #2: BLQ |
| 8 | Subj. #3: BLQ | Subj. #4: BLQ |

As used herein, the terms “about” or “approximately” are taken to mean±10%, ±5%, ±2.5% or ±1% of a specified valued. As used herein, the term “substantially” is taken to mean “to a large degree” or “at least a majority of” or “more than 50% of”

The above is a detailed description of particular embodiments of the invention. It will be appreciated that, although specific embodiments of the invention have been described herein for purposes of illustration, various modifications may be made without departing from the spirit and scope of the invention. Accordingly, the invention is not limited except as by the appended claims. All of the embodiments disclosed and claimed herein can be made and executed without undue experimentation in light of the present disclosure.

1) A method of treating viral infection in an animal in need thereof, the method comprising administering to the animal one or more doses of an antiviral composition comprising oleandrin, digoxin, or a combination thereof, wherein the viral infection excludes equine encephalitis.

2) A method of preventing viral infection in an animal at risk of contracting said viral infection, the method comprising chronically administering to the animal one or more doses of an antiviral composition on a recurring or continuous basis over an extended treatment period prior to the animal contracting the viral infection, thereby preventing the animal from contracting said viral infection, wherein the antiviral composition comprises oleandrin, digoxin, or a combination thereof, wherein the viral infection excludes equine encephalitis.

3) A method of preventing an animal from exhibiting one or more symptoms associated with viral infection, the method comprising administering to said animal one or more therapeutically effective doses of cardiac glycoside-containing composition, wherein said one or more doses are administered a) prior to said animal being infected with virus; or b) within a period of up to five days, up to four days, up to three days, up to two days, or up to one day of said animal having been infected with virus, wherein the viral infection excludes equine encephalitis.

4) (canceled)

5) (canceled)

6) (canceled)

7) The method of claim 1, wherein the antiviral composition is administered systemically.

8) The method of claim 2, wherein the animal has been in close contact with another animal having a viral infection, and/or wherein the uninfected animal has been living with, sharing food with, sharing shelter with, sharing air with, or sharing water with a virally infected animal.

9) The method of claim 1, wherein
a) said animal is a cow and the dose of antiviral composition provides a maximum plasma concentration of digoxin or oleandrin of no more than 1 ng/mL;
b) said animal is a pig and the dose of antiviral composition provides a maximum plasma concentration of digoxin or oleandrin of no more than 5 ng/mL;
c) said animal is a horse and the dose of antiviral composition provides a maximum plasma concentration of digoxin or oleandrin of no more than 5 ng/mL;
d) said animal is a sheep and the dose of antiviral composition provides a maximum plasma concentration of digoxin or oleandrin of no more than 5 ng/mL;
e) said animal is a goat and the dose of antiviral composition provides a maximum plasma concentration of digoxin or oleandrin of no more than 10 ng/mL;
f) said animal is a god and the dose of antiviral composition provides a maximum plasma concentration of digoxin or oleandrin of no more than 5 ng/mL.

10) The method of claim 1, wherein a dose of said antiviral composition comprises about 0.05-0.5 microg/kg/day, about 0.05-0.35 microg/kg/day, about 0.05-0.22 microg/kg/day, about 0.05-0.4 microg/kg/day, or about 0.05-0.3 microg/kg/day, based upon the unit amount of oleandrin and/or digoxin per kg of bodyweight per day.

11) The method of claim 1, wherein the cardiac glycoside is administered in at least two dosing phases: a loading phase and a maintenance phase.

12) The method of claim 1, wherein following administration of said one or more doses, the plasma concentration of oleandrin in said subject is in the range of about 0.05 to about 2 ng/ml, about 0.005 to about 10 ng/mL, about 0.005 to about 8 ng/mL, about 0.01 to about 7 ng/mL, about 0.02 to about 7 ng/mL, about 0.03 to about 6 ng/mL, about 0.04 to about 5 ng/mL, or about 0.05 to about 2.5 ng/mL, in terms of the amount of oleandrin per mL of plasma.

13) The method of claim 1, wherein a) plural doses are one or more doses administered per day for two or more days per week; b) 1-10 doses of cardiac glycoside (cardiac glycoside-containing composition) per day are administered for a treatment period of 2 days to about 2 months; or c) one or more doses of cardiac glycoside (cardiac glycoside-containing composition) are administered per day for plural days and plural weeks until the viral infection is cured.

14) The method of claim 13, wherein dosing is continued for one or more weeks per month.

15) The method of claim 14, wherein dosing is continued for one or more months per year.

16) The method of claim 1, wherein said antiviral composition comprises a) oleandrin; b) a combination of oleandrin, oleanolic acid (free acid, salt, or prodrug) and ursolic acid (free acid, salt, or prodrug); c) a combination of oleandrin, oleanolic acid (free acid, salt, or prodrug) and betulinic acid (free acid, salt, or prodrug); d) a combination of oleandrin, oleanolic acid (free acid, salt, or prodrug), ursolic acid (free acid, salt, or prodrug), and betulinic acid (free acid, salt, or prodrug); e) a combination of oleandrin, oleanolic acid (free acid or salt thereof), ursolic acid (free acid or salt), and betulinic acid (free acid or); f) a combination of oleandrin and at least two triterpenes selected from the group consisting of oleanolic acid (free acid, salt, or prodrug), ursolic acid (free acid, salt, or prodrug), betulinic acid (free acid, salt, or prodrug); or g) a combination of at least oleandrin, oleanolic acid, ursolic acid, betulinic acid, kanerocin, kanerodione, oleandrigenin, Nerium F, neritaloside, odoroside, adynerin, odoroside-G-acetate, and gitoxigenin.

17) The method of claim 1, wherein said antiviral composition further comprises polyphenol(s), carbohydrate(s), flavonoid(s), amino acid(s), soluble protein(s), cellulose, starch, alkaloid(s), saponin(s), tannin(s), or any combination thereof.

18) The method of claim 1, wherein said antiviral composition comprises an extract of biomass.

19) The method of claim 18, wherein said extract is prepared by hot-water extraction, cold-water extraction, organic solvent extraction, supercritical fluid extraction, subcritical liquid extraction, or a combination thereof.

20) The method of claim 18, wherein said extract comprises a combination of oleandrin and one or more compounds extracted from said biomass.

21) The method of claim 20, wherein said biomass is plant material from Nerium species or Agrobacterium species biomass.

22) The method of claim 20, wherein said extract further comprises one or more cardiac glycoside precursors, one or more glycone constituents of cardiac glycosides, or a combination thereof.

23) The method of claim 22, wherein said extract comprises oleandrin and one or more compounds selected from the group consisting of cardiac glycoside, glycone, aglycone, steroid, triterpene, polysaccharide, saccharide, alkaloid, fat, protein, neritaloside, odoroside, oleanolic acid, ursolic acid, betulinic acid, oleandrigenin, oleaside A, betulin (urs-12-ene-3β,28-diol), 28-norurs-12-en-3β-ol, urs-12-en-3β-ol, 3β,3β-hydroxy-12-oleanen-28-oic acid, 3β,20α-dihydroxyurs-21-en-28-oic acid, 3β,27-dihydroxy-12-ursen-28-oic acid, 3β,13β-dihydroxyurs-11-en-28-oic acid, 3β,12α-dihydroxyoleanan-28,13β-olide, 3β,27-dihydroxy-12-oleanan-28-oic acid, homopolygalacturonan, arabinogalaturonan, chlorogenic acid, caffeic acid, L-quinic acid, 4-coumaroyl-CoA, 3-O-caffeoylquinic acid, 5-O-caffeoylquinic acid, cardenolide B-1, cardenolide B-2, oleagenin, neridiginoside, nerizoside, odoroside-H, 3-beta-O-(D-diginosyl)-5-beta, 14 beta-dihydroxy-card-20(22)-enolide pectic polysaccharide composed of galacturonic acid, rhamnose, arabinose, xylose, and galactose, polysaccharide with MW in the range of 17000-120000 D, or MW about 35000 D, about 3000 D, about 5500 D, or about 12000 D, cardenolide monoglycoside, cardenolide N-1, cardenolide N-2, cardenolide N-3, cardenolide N-4, pregnane, 4,6-diene-3,12,20-trione, 20R-hydroxypregna-4,6-diene-3,12-dione, 16beta,17beta-epoxy-12beta-hydroxypregna-4,6-diene-3,20-dione, 12beta-hydroxypregna-4,6,16-triene-3,20-dione (neridienone A), 20S,21-dihydroxypregna-4,6-diene-3,12-dione (neridienone B), neriucoumaric acid, isoneriucoumaric acid, oleanderoic acid, oleanderen, 8alpha-methoxylabdan-18-oic acid, 12-ursene, kaneroside, neriumoside, 3β-O-(D-diginosyl)-2α-hydroxy-8,14β-epoxy-5β-carda-16:17, 20:22-dienolide, 3β-O-(D-diginosyl)-2α,14β-dihydroxy-5β-carda-16:17,20:22-dienolide, 3β,27-dihydroxy-urs-18-en-13,28-olide, 3β,22α,28-trihydroxy-25-nor-lup-1(10),20(29)-dien-2-one, cis-karenin (3β-hydroxy-28-Z-p-coumaroyloxy-urs-12-en-27-oic acid), trans-karenin (3-β-hydroxy-28-E-p-coumaroyloxy-urs-12-en-27-oic acid), 3beta-hydroxy-5alpha-carda-14(15),20(22)-dienolide (beta-anhydroepidigitoxigenin), 3 beta-O-(D-digitalosyl)-21-hydroxy-5beta-carda-8,14,16,20(22)-tetraenolide (neriumogenin-A-3beta-D-digitaloside), proceragenin, neridienone A, 3beta,27-dihydroxy-12-ursen-28-oic acid, 3beta,13beta-dihydroxyurs-II-en-28-oic acid, 3beta-hydroxyurs-12-en-28-aldehyde, 28-orurs-12-en-3beta-ol, urs-12-en-3beta-ol, urs-12-ene-3beta,28-diol, 3beta,27-dihydroxy-12-oleanen-28-oic acid, (20S,24R)-epoxydammarane-3beta,25-diol, 20beta,28-epoxy-28alpha-methoxytaraxasteran-3beta-ol, 20beta,28-epoxytaraxaster-21-en-3beta-ol, 28-nor-urs-12-ene-3beta,17 beta-diol, 3beta-hydroxyurs-12-en-28-aldehyde, alpha-neriursate, beta-neriursate, 3alpha-acetophenoxy-urs-12-en-28-oic acid, 3beta-acetophenoxy-urs-12-en-28-oic acid, oleanderolic acid, kanerodione, 3β-p-hydroxyphenoxy-11α-methoxy-12α-hydroxy-20-ursen-28-oic acid, 28-hydroxy-20(29)-lupen-3,7-dione, kanerocin, 3alpha-hydroxy-urs-18,20-dien-28-oic acid, D-sarmentose, D-diginose, neridiginoside, nerizoside, isoricinoleic acid, gentiobiosylnerigoside, gentiobiosylbeaumontoside, gentiobiosyloleandrin, folinerin, 12β-hydroxy-5β-carda-8,14,16,20(22)-tetraenolide, 8β-hydroxy-digitoxigenin, Δ16-8β-hydroxy-digitoxigenin, Δ16-neriagenin, uvaol, ursolic aldehyde, 27(p-coumaroyloxy)ursolic acid, oleanderol, 16-anhydro-deacteyl-nerigoside, 9-D-hydroxy-cis-12-octadecanoic acid, adigoside, adynerin, alpha-amyrin, beta-sitosterol, campestrol, caoutchouc, capric acid, caprylic acid, choline, cornerin, cortenerin, deacetyloleandrin, diacetyl-nerigoside, foliandrin, pseudocuramine, quercetin, quercetin-3-rhamnoglucoside, quercitrin, rosaginin, rutin, stearic acid, stigmasterol, strospeside, urehitoxin, and uzarigenin.

24) The method of claim 1, wherein the viral infection is selected from the group consisting of bovine coronavirus (BCV), porcine coronavirus (PCV), bovine viral diarrhea virus (BVDV), bovine respiratory syncytial virus (BRSV), and porcine reproductive and respiratory syndrome virus (PRRSV).

25) The method of claim 1, wherein the viral infection is selected from the group consisting of porcine circovirus type-2 (PCV2), bovine herpes virus type 1 (BHV-1, e.g. infectious bovine rhinotracheitis (IBR)), bovine herpes virus type 2 (BHV-2, bovine herpes mamillitis), bovine herpes virus type 3 (BHV-3, catarrhal fever), bovine herpes virus type 5 (BHV-5, encephalitis), bovine papillomavirus, lyssavirus (rabies, a Rhabdovirus), Foot and Mouth Disease virus (FMD; aphthovirus of the family Picornaviridae; e.g. serotypes A, O, C, SAT1,SAT2, SAT3, Asia1), lumpy skin disease virus (Capripoxvirus of the Poxviridae family), cowpox virus, pseudocowpox virus (paravaccinia), bovine leukemia virus, bovine lentivirus, respirovirus (bovine parainfluenza-3 virus), Morbillivirus (rinderpest virus), bovine ephemeral fever virus, vesicular stomatitis virus, African swine fever virus, African horse sickness virus (Reoviridae), sheeppox virus and goatpox virus (subfamily Chordopoxviridae, genus Capripoxvirus), equine influenza virus, equine infectious anemia virus, equine arteritis virus, classical swine fever virus, Nipah virus, swine vesicular disease virus, transmissible gastroenteritis virus of swine, avian infectious bronchitis virus, infectious laryngotracheitis virus (avian), duck hepatitis virus, avian influenza virus, infectious bursal disease virus (Gumboro), Marek's disease virus (visceral leukosis; Herpes virus), virulent Newcastle disease virus (vNDV, Paramyxoviridae, genus Avulavirus), avian metapneumovirus (in turkey), avian influenza virus, Poult Enteritis Mortality Syndrome (PEMS in turkey), columbid alphaherpesvirus-1 (CoHV-1), avian nephritis, arbovirus infections, turkey viral hepatitis, avian encephalomyelitis, avian hepatitis E virus, chicken cholera, fowl pox, fowl cholera, hemorrhagic enteritis in turkeys, canine parvovirus type 1 or type 2, infectious canine hepatitis (ICH, adenovirus 1), canine herpes, canine distemper virus (Morbillivirus), rotavirus intestinal viral in dogs, porcine herpesvirus 1 (pseudorabies, Aujeszky's disease), canine influenza, canine parainfluenza virus, feline herpes virus, feline immunodeficiency virus, feline parvovirus, feline infectious peritonitis virus, feline influenza virus, feline calicivirus, feline leukemia virus, feline viral rhinotracheitis, feline coronavirus, feline rotavirus, feline astrovirus, Torque teno sus virus (TTSuV), Porcine teschovirus (PTV), Porcine bocavirus 1 (PBoV1), swine influenza virus (e.g. type A), porcine endemic diarrhea virus (PEDV), porcine deltacoronavirus, species thereof, and variants thereof.

26) The method of claim 1, wherein the animal is selected from the group consisting of pig, cow, horse, sheep, goat, llama, alpaca, buffalo, deer, elk, giraffe, camel, dog, cat, chicken, turkey, pigeon, duck, pheasant, and guinea.

27) The method of claim 1, wherein the antiviral composition further comprises at least one inhibitor that reduces the rate of metabolism or digestion of the cardiac glycoside, thereby increasing the plasma concentration half-life of the cardiac glycoside in the animal.

28) The method of claim 27, wherein said inhibitor inhibits metabolism or digestion of said cardiac glycoside.

29) The method of claim 1, wherein said antiviral composition is included in a feed and/or liquid administered orally to the animal.

30) (canceled)

31) (canceled)

32) (canceled)

33) (canceled)

34) (canceled)
